(19) United States
(12) Patent Application Publication
(10) Pub. No.: US 2002/0102640 A1
(43) Pub. Date: Aug. 1, 2002

(54) 83P5G4: a tissue specific protein highly expressed in prostate cancer

(76) Inventors: Rene S. Hubert, Los Angeles, CA (US); Daniel E.H. Afar, Brisbane, CA (US); Pia M. Challita-Eid, Encino, CA (US); Mary Faris, Los Angeles, CA (US); Elana Levin, Los Angeles, CA (US); Steve Chappell Mitchell, Santa Monica, CA (US); Aya Jakobovits, Beverly Hills, CA (US)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 09/780,053

(22) Filed: Feb. 9, 2001

Related U.S. Application Data
(60) Provisional application No. 60/181,261, filed on Feb. 9, 2000.

(51) Int. Cl. C12P 21/02; C07H 21/04

(52) U.S. Cl. 435/69.1; 536/23.2; 435/320.1

(57) ABSTRACT

A novel gene (designated 83P5G4) and its encoded protein are described. Whereas 83P5G4 exhibits tissue specific expression in normal adult tissue, it is aberrantly expressed multiple cancers including prostate, testicular, bladder, kidney, brain, bone, cervical, uterine, ovarian, breast, pancreatic, stomach, colon, rectal, leukocytic, liver and lung cancers. Consequently, 83P5G4 provides a diagnostic and/or therapeutic target for cancers, and the 83P5G4 gene or fragment thereof, or its encoded protein or a fragment thereof used to elicit an immune response.

### CROSS REFERENCE TO RELATED APPLICATIONS

This application claims the benefit of U.S. provisional patent application No. 60/181,261, filed Feb. 9, 2000, the entire contents of which are incorporated herein by reference.

### FIELD OF THE INVENTION

The invention described herein relates to a novel gene and its encoded protein, termed 83P5G4, and to diagnostic and therapeutic methods and compositions useful in the management of various cancers that express 83P5G4, particularly prostate cancers.

### BACKGROUND OF THE INVENTION

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise. In the early part of the next century, cancer is predicted to become the leading cause of death.

Worldwide, several cancers stand out as the leading killers. In particular, carcinomas of the lung, prostate, breast, colon, pancreas, and ovary represent the primary causes of cancer death. These and virtually all other carcinomas share a common lethal feature. With very few exceptions, metastatic disease from a carcinoma is fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients experience physical debilitations following treatment. Furthermore, many cancer patients experience a recurrence.

Worldwide, prostate cancer is the fourth most prevalent cancer in men. In North America and Northern Europe, it is by far the most common cancer in males and is the second leading cause of cancer death in men. In the United States alone, well over 40,000 men die annually of this disease—second only to lung cancer. Despite the magnitude of these figures, there is still no effective treatment for metastatic prostate cancer. Surgical prostatectomy, radiation therapy, hormone ablation therapy, surgical castration and chemotherapy continue to be the main treatment modalities. Unfortunately, these treatments are ineffective for many and are often associated with undesirable consequences.

On the diagnostic front, the lack of a prostate tumor marker that can accurately detect early-stage, localized tumors remains a significant limitation in the diagnosis and management of this disease.

Although the serum prostate specific antigen (PSA) assay has been a very useful tool, however its specificity and general utility is widely regarded as lacking in several important respects.

Progress in identifying additional specific markers for prostate cancer has been improved by the generation of prostate cancer xenografts that can recapitulate different stages of the disease in mice. The LAPC (Los Angeles Prostate Cancer) xenografts are prostate cancer xenografts that have survived passage in severe combined immune deficient (SCID) mice and have exhibited the capacity to mimic the transition from androgen dependence to androgen independence (Klein et al., 1997, Nat. Med.3:402). More recently identified prostate cancer markers include PCTA-1 (Su et al., 1996, Proc. Natl. Acad. Sci. USA 93:7252), prostate-specific membrane (PSM) antigen (Pinto et al., Clin Cancer Res 1996 Sep; 2(9):1445-51), STEAP (Proc Natl Acad Sci U S A. 1999 Dec 7; 96(25): 14523-8) and prostate stem cell antigen (PSCA) (Reiter et al., 1998, Proc. Natl. Acad. Sci. USA 95:1735).

While previously identified markers such as PSA, PSM, PCTA and PSCA have facilitated efforts to diagnose and treat prostate cancer, there is need for the identification of additional markers and therapeutic targets for prostate and related cancers in order to further improve diagnosis and therapy.

### SUMMARY OF THE INVENTION

The present invention relates to a novel gene, designated 83P5G4 that is highly expressed in multiple cancers listed in Table I. Northern blot expression analysis of 83P5G4 gene expression in normal tissues shows expression of 1.8, 2.5 and 4.5 kb transcripts in multiple tissues. Northern blot analysis suggests that different tissues express different mRNA isoforms of 83P5G4 and the 83P5G4 mRNA isoforms in prostate cancer appear to be different from the mRNA isoform expressed in normal prostate. The nucleotide (SEQ ID NO: 1) and amino acid (SEQ ID NO: 2) sequences of 83P5G4 are shown in FIG. 2. Portions of the 83P5G4 amino acid sequence show some homologies to ESTs in the dbEST database. The expression profile of 83P5G4 in normal adult tissues, combined with the expression observed in cancer cells such as prostate tumor xenografts, provides evidence that 83P5G4 is aberrantly expressed in at least some cancers such as prostate cancer, and can serve as a useful diagnostic and/or therapeutic target for cancers of the tissues listed in Table I (see, e.g., FIGS. 4-9).

The invention provides polynucleotides corresponding or complementary to all or part of the 83P5G4 genes, mRNAs, and/or coding sequences, preferably in isolated form, including polynucleotides encoding 83P5G4 proteins and fragments of 4, 5, 6, 7, 8, 9, 10, 11, 12, 13, 14, 15 or more amino acids as well as the peptides/proteins themselves, DNA, RNA, DNA/RNA hybrids, and related molecules, polynucleotides or oligonucleotides complementary or having at least a 90% homology to the 83P5G4 genes or mRNA sequences or parts thereof, and polynucleotides or oligonucleotides that hybridize to the 83P5G4 genes, mRNAs, or to 83P5G4-encoding polynucleotides. Also provided are means for isolating cDNAs and the genes encoding 83P5G4. Recombinant DNA molecules containing 83P5G4 polynucleotides, cells transformed or transduced with such molecules, and host-vector systems for the expression of 83P5G4 gene products are also provided. The invention further provides antibodies that bind to 83P5G4 proteins and polypeptide fragments thereof, including polyclonal and monoclonal antibodies, murine and other mammalian antibodies, chimeric antibodies, humanized and fully human antibodies, and antibodies labeled with a detectable marker.

The invention further provides methods for detecting the presence and status of 83P5G4 polynucleotides and proteins in various biological samples, as well as methods for identifying cells that express 83P5G4. A typical embodiment of this invention provides methods for monitoring 83P5G4 gene products in a tissue or hematology sample having or suspected of having some form of growth disregulation such as cancer.

The invention further provides various immunogenic or therapeutic compositions and strategies for treating cancers that express 83P5G4 such as prostate cancers, including therapies aimed at inhibiting the transcription, translation, processing or function of 83P5G4 as well as cancer vaccines.

### BRIEF DESCRIPTION OF THE FIGURES

FIG. 1. shows the 83P5G4 suppression subtractive hybridization (SSH) DNA sequence of 445 nucleotides in length (SEQ ID NO: 3).

FIG. 2. shows the nucleotide (SEQ ID NO: 1) and amino acid (SEQ ID NO: 2) sequences of 83P5G4.

FIG. 3. shows the sequence alignment of 83P5G4 with the Drosophila lethal (2) denticless (L2DT) using the BLAST function (NCBI). The proteins are 42% identical and 60% homologous over a 352 a.a. region. The WD repeat domains are bolded in the L2DT sequence. Score=294 bits (745), Expect=1e-78. Identities=149/352 (42%), Positives=215/352 (60%), Gaps =6/352 (1%)

FIGS. 4A-4C. show 83P5G4 expression in various normal human tissues (using the 83P5G4 SSH fragment as a probe) and LAPC xenografts. Two multiple tissue Northern blots (Clontech) (FIGS. 4A and 4B) and a xenograft Northern blot (FIG. 4C) were probed with the 83P5G4 SSH fragment. Lanes 1-8 in FIG. 4A consist of mRNA from heart, brain, placenta, lung, liver, skeletal muscle, kidney and pancreas respectively. Lanes 1-8 in FIG. 4B consist of mRNA from spleen, thymus, prostate, testis, ovary, small intestine, colon and leukocytes respectively. Lanes 1-5 in FIG. 4C consist of total RNA from prostate cancer xenografts, LAPC-4 AD, LAPC-4 AI, LAPC-9 AD and LAPC-9 Al respectively. Size standards in kilobases (kb) are indicated on the side. Each lane contains 2 μg of mRNA for the normal tissues and 10 μg of total RNA for the xenograft tissues. The results show the tissue specific expression of 1.8, 2.5 and/or 4.5 kb 83P5G4 transcripts in multiple tissues.

FIG. 5. shows a Northern blot analysis of 83P5G4 expression in prostate cancer xenografts. Lanes 1-14 show LAPC-4 AD sc, LAPC-4 AD sc, LAPC-4 AD sc, LAPC-4 AD it, LAPC-4 AD it, LAPC-4 AD it, LAPC-4 AD 2, LAPC-9 AD sc, LAPC-9 AD sc, LAPC-9 AD it, LAPC-9 AD it, LAPC-9 AD it, LAPC-3 Al sc and LAPC-3 AI sc respectively.

FIG. 6. shows the Northern blot analysis of 83P5G4 expression in prostate and multiple cancer cell lines. Lanes 1-56 show expression in LAPC-4 AD, LAPC-4 Al, LAPC-9 AD, LAPC-9 Al, TSUPR-1, DU145, LNCaP, PC-3, LAPC-4 CL, PrEC, HT1197, SCaBER, UM-UC-3, TCCSUP, J82, 5637, 293T, RD-ES, PANC-1, BxPC-3, HPAC, Capan-1, CaCo-2, LoVo, T84, Colo-205, KCL 22, PFSK-1, T98G, SK-ES-1, HOS, U2-OS, RD-ES, CALU-1, A427, NCI-H82, NCI-H146, 769-P, A498, CAKI-1, SW839, BT20, CAMA-1, DU4475, MCF-7, MDA-MB-435s, NTERRA-2, NCCIT, TERA-1, TERA-2, A431, HeLa, OV-1063, PA-1, SW626 and CAOV-3 respectively.

FIG. 7. shows the Northern blot analysis of 83P5G4 expression in prostate cancer patient samples. Lanes 1-8 show Normal prostate, Patient 1 normal adjacent tissue, Patient 1 Gleason 9 tumor, Patient 2 normal adjacent tissue, Patient 2 Gleason 7 tumor, Patient 3 normal adjacent tissue and Patient 3 Gleason 7 tumor respectively.

FIG. 8. Shows expression of 83P5G4 assayed in a panel of human tumors (T) and their respective matched normal tissues (N) on RNA dot blots. 83P5G4 expression was seen in kidney, breast, prostate, uterus, ovary, cervix, colon, lung, stomach, rectum, and small intestine cancers. 83P5G4 was also found to be highly expressed in all nine cell lines tested (from left to right); HeLa (cervical carcinoma, Daudi (Burkitt's lymphoma), K562 (CML), HL-60 (PML), G361 (melanoma), A549 (lung carcinoma), MOLT-4 (lymphoblastic leukemia), SW480 (colorectal carcinoma), Raji (Burkitt's lymphoma). The expression detected in normal adjacent tissues (isolated from diseased tissues), but not in normal tissues (isolated from healthy donors), indicates that these tissues are not truly normal and that 83P5G4 is expressed in early stage tumors.

FIG. 9 shows a RT-PCR Expression analysis of 83P5G4. cDNAs generated from pools of tissues from multiple normal and cancer tissues were normalized using beta-actin primers, and used to study the expression of 83P5G4. Aliquots of the RT-PCR mix after 30 cycles were run on the agarose gel to allow semi-quantitative evaluation of the levels of expression between samples. Lane 1 (VP-1) contains liver, lung, and kidney first strand cDNA; lane 2 (VP-2) stomach, spleen, and pancreas; lane 3 (xenograft pool) LAPC4AD, LAPC4AI, LAPC9AD, and LAPC9AI; lane 4 is bladder cancer pool; lane 5 is kidney cancer pool; lane 6 is colon cancer pool; lane 7 is from a lung cancer patient; and lane 8 is a water blank.

FIG. 10 shows the amino acid sequence of 83P5G4.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

### DEFINITIONS

As used herein, the terms “advanced prostate cancer”, “locally advanced prostate cancer”, “advanced disease” and “locally advanced disease” mean prostate cancers that have extended through the prostate capsule, and are meant to include stage C disease under the American Urological Association (AUA) system, stage C1-C2 disease under the Whitmore-Jewett system, and stage T3 - T4 and N+ disease under the TNM (tumor, node, metastasis) system. In general, surgery is not recommended for patients with locally advanced disease, and these patients have substantially less favorable outcomes compared to patients having clinically localized (organ-confined) prostate cancer. Locally advanced disease is clinically identified by palpable evidence of induration beyond the lateral border of the prostate, or asymmetry or induration above the prostate base. Locally advanced prostate cancer is presently diagnosed pathologically following radical prostatectomy if the tumor invades or penetrates the prostatic capsule, extends into the surgical margin, or invades the seminal vesicles.

“Altering the native glycosylation pattern” is intended for purposes herein to mean deleting one or more carbohydrate moieties found in native sequence 83P5G4 (either by removing the underlying glycosylation site or by deleting the glycosylation by chemical and/or enzymatic means), and/or adding one or more glycosylation sites that are not present in the native sequence 83P5G4. In addition, the phrase includes qualitative changes in the glycosylation of the native proteins, involving a change in the nature and proportions of the various carbohydrate moieties present.

The term “analog” refers to a molecule that is structurally similar or shares similar or corresponding attributes with another molecule (e.g. a 83P5G4-related protein). The term “homolog” refers to a molecule which exhibits homology to another molecule, by for example, having sequences of chemical residues that are the same or similar at corresponding positions.

The term “antibody” is used in the broadest sense. Therefore an “antibody” can be naturally occurring or man made such as monoclonal antibodies produced by conventional hybridoma technology. Anti-83P5G4 antibodies comprise monoclonal and polyclonal antibodies as well as fragments containing the antigen-binding domain and/or one or more complementarity determining regions of these antibodies. As used herein, an antibody fragment is defined as at least a portion of the variable region of the immunoglobulin molecule that binds to its target, i.e., the antigen-binding region. In one embodiment it specifically covers single anti-83P5G4 antibody (including agonist, antagonist and neutralizing antibodies) and anti-83P5G4 antibody compositions with polyepitopic specificity. The term “monoclonal antibody” as used herein refers to an antibody obtained from a population of substantially homogeneous antibodies, i.e., the antibodies comprising the population are identical except for possible naturally-occurring mutations that are present in minor amounts.

The term “codon optimized sequences” refers to nucleotide sequences that have been optimized for a particular host species by replacing any codons having a usage frequency of less than about 20%. Nucleotide sequences that have been optimized for expression in a given host species by elimination of spurious polyadenylation sequences, elimination of exon/intron splicing signals, elimination of transposon-like repeats and/or optimization of GC content in addition to codon optimization are referred to herein as an “expression enhanced sequences.”

The term “cytotoxic agent” as used herein refers to a substance that inhibits or prevents the function of cells and/or causes destruction of cells. The term is intended to include radioactive isotopes chemotherapeutic agents, and toxins such as small molecule toxins or enzymatically active toxins of bacterial, fungal, plant or animal origin, including fragments and/or variants thereof. Examples of cytotoxic agents include, but are not limited to maytansinoids, ytrium, bismuth ricin, ricin A-chain, doxorubicin, daunorubicin, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, sapaonaria officinalis inhibitor, and glucocorticoid and other chemotherapeutic agents, as well as radioisotopes such as At²¹¹, I¹³¹, I¹²⁵, Y⁹⁰, Re¹⁸⁶, Re¹⁸⁸, Sm¹⁵³, Bi²¹², P³² and radioactive isotopes of Lu. Antibodies may also be conjugated to an anti-cancer pro-drug activating enzyme capable of converting the pro-drug to its active form.

As used herein, the terms “hybridize”, “hybridizing”, “hybridizes” and the like, used in the context of polynucleotides, are meant to refer to conventional hybridization conditions, preferably such as hybridization in 50% formamide/6×SSC/0.1% SDS/100 μg/ml ssDNA, in which temperatures for hybridization are above 37 degrees C and temperatures for washing in 0.1×SSC/0.1% SDS are above 55 degrees C.

As used herein, a polynucleotide is said to be “isolated” when it is substantially separated from contaminant polynucleotides that correspond or are complementary to genes other than the 83P5G4 gene or that encode polypeptides other than 83P5G4 gene product or fragments thereof. A skilled artisan can readily employ nucleic acid isolation procedures to obtain an isolated 83P5G4 polynucleotide.

As used herein, a protein is said to be “isolated” when physical, mechanical or chemical methods are employed to remove the 83P5G4 protein from cellular constituents that are normally associated with the protein. A skilled artisan can readily employ standard purification methods to obtain an isolated 83P5G4 protein.

The term “mammal” as used herein refers to any mammal classified as a mammal, including mice, rats, rabbits, dogs, cats, cows, horses and humans. In one preferred embodiment of the invention, the mammal is a mouse. In another preferred embodiment of the invention, the mammal is a human.

As used herein, the terms “metastatic prostate cancer” and “metastatic disease” mean prostate cancers that have spread to regional lymph nodes or to distant sites, and are meant to include stage D disease under the AUA system and stage TxNxM+ under the TNM system. As is the case with locally advanced prostate cancer, surgery is generally not indicated for patients with metastatic disease, and hormonal (androgen ablation) therapy is a preferred treatment modality. Patients with metastatic prostate cancer eventually develop an androgen-refractory state within 12 to 18 months of treatment initiation, and approximately half of these patients die within 6 months after developing androgen refractory status. The most common site for prostate cancer metastasis is bone. Prostate cancer bone metastases are often characteristically osteoblastic rather than osteolytic (i.e., resulting in net bone formation). Bone metastases are found most frequently in the spine, followed by the femur, pelvis, rib cage, skull and humurus. Other common sites for metastasis include lymph nodes, lung, liver and brain. Metastatic prostate cancer is typically diagnosed by open or laparoscopic pelvic lymphadenectomy, whole body radionuclide scans, skeletal radiography, and/or bone lesion biopsy. “Moderately stringent conditions” are described by, identified but not limited to, those in Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

As used herein “motif” as in biological motif of an 83P5G4-related protein, refers to any set of amino acids forming part of the primary sequence of a protein, either contiguous or capable of being aligned to certain positions that are generally invariant or conserved, that is associated with a particular function or modification (e.g. that is phosphorylated, glycosylated or amidated), or a sequence that is correlated with being immunogenic, either humorally or cellularly.

As used herein, the term “polynucleotide” means a polymeric form of nucleotides of at least 10 bases or base pairs in length, either ribonucleotides or deoxynucleotides or a modified form of either type of nucleotide, and is meant to include single and double stranded forms of DNA and/or RNA. In the art, this term if often used interchangeably with “oligonucleotide”. As discussed herein, a polynucleotide can comprise a nucleotide sequence disclosed herein wherein thymidine (T) (as shown for example in SEQ ID NO: 1) can also be uracil (U). This description pertains to the differences between the chemical structures of DNA and RNA, in particular the observation that one of the four major bases in RNA is uracil (U) instead of thymidine (T).

As used herein, the term “polypeptide” means a polymer of at least about 4, 5, 6, 7, or 8 amino acids. Throughout the specification, standard three letter or single letter designations for amino acids are used. In the art, this term if often used interchangeably with “peptide”.

As used herein, a “recombinant” DNA or RNA molecule is a DNA or RNA molecule that has been subjected to molecular manipulation in vitro.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, are identified by, but not limited to, those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (PH 6.8), 0.1% sodium pyrophosphate, 5×Denhardt's solution, sonicated salmon sperm DNA (50 [g/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

A “transgenic animal” (e.g., a mouse or rat) is an animal having cells that contain a transgene, which transgene was introduced into the animal or an ancestor of the animal at a prenatal, e.g., an embryonic stage. A “transgene” is a DNA that is integrated into the genome of a cell from which a transgenic animal develops.

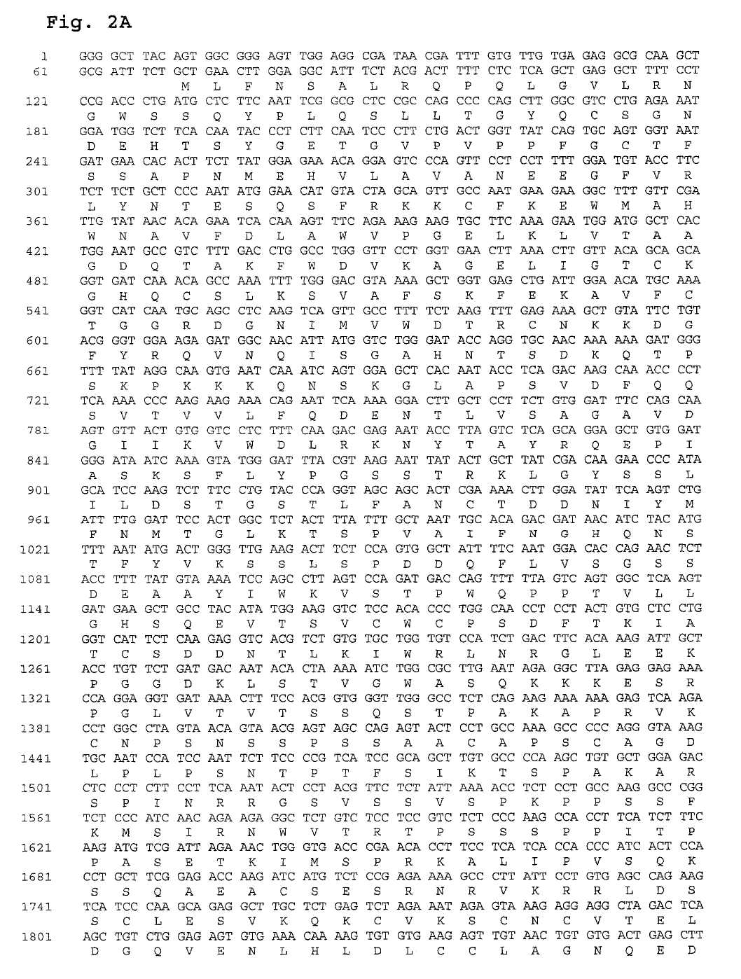

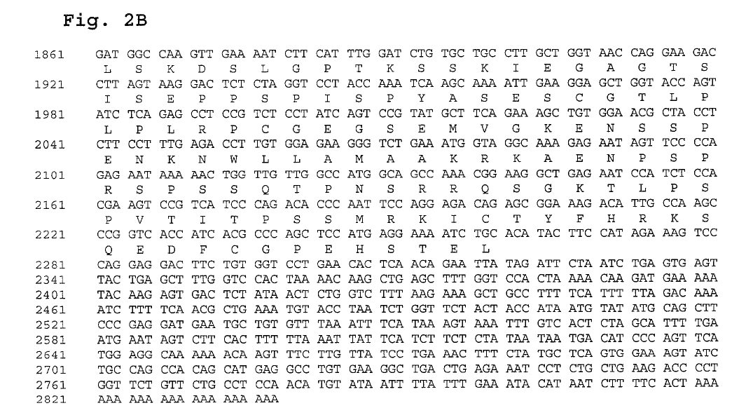

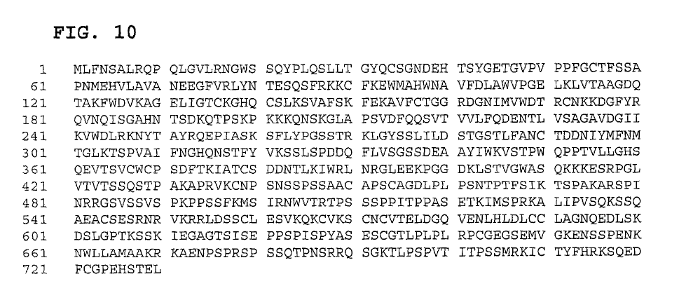

The term “variant” refers to a molecule that exhibits a variation from a described type or norm, such as a protein that has one or more different amino acid residues in the corresponding position(s) of a specifically described protein (e.g. the 83P5G4 protein shown, e.g., in FIG. 2 and FIG. 10).

As used herein, the 83P5G4 gene and protein is meant to include the 83P5G4 genes and proteins specifically described herein and the genes and proteins corresponding to other 83P5G4 encoded proteins or peptides and structurally similar variants of the foregoing. Such other 83P5G4 peptides and variants will generally have coding sequences that are highly homologous to the 83P5G4 coding sequence, and preferably share at least about 50% amino acid homology (using BLAST criteria) and preferably 50%, 60%, 70%, 80%, 90% or more nucleic acid homology, and at least about 60% amino acid homology (using BLAST criteria), more preferably sharing 70% or greater homology (using BLAST criteria).

The 83P5G4-related proteins of the invention include those specifically identified herein, as well as allelic variants, conservative substitution variants, analogs and homologs that can be isolated/generated and characterized without undue experimentation following the methods outlined herein or are readily available in the art. Fusion proteins that combine parts of different 83P5G4 proteins or fragments thereof, as well as fusion proteins of an 83P5G4 protein and a heterologous polypeptide are also included. Such 83P5G4 proteins are collectively referred to as the 83P5G4-related proteins, the proteins of the invention, or 83P5G4. As used herein, the term “83P5G4-related protein” refers to a polypeptide fragment or a 83P5G4 protein sequence of 4, 5, 6, 7, 8, 9, 10, 11, 12, 13, 14, 15 or more amino acids.

### CHARACTERIZATION OF 83P5G4

As discussed in detail herein, experiments with the LAPC-4 AD xenograft in male SCID mice have resulted in the identification of genes that are involved in the progression of androgen dependent (AD) prostate cancer to androgen independent (Al) cancer. Briefly, to isolate genes that are involved in the progression of androgen dependent (AD) prostate cancer to androgen independent (Al) cancer we conducted an experiment with the LAPC-4 AD xenograft in male SCID mice. Mice that harbored LAPC-4 AD xenografts were castrated when the tumors reached a size of 1 cm in diameter. The tumors stopped growing and temporarily stopped producing the androgen dependent protein PSA. Seven to fourteen days post-castration, PSA levels were detectable again in the blood of the mice. Eventually the tumors develop an Al phenotype and start growing again in the castrated males. Tumors were harvested at different time points after castration to identify genes that are turned on or off during the transition to androgen independence.

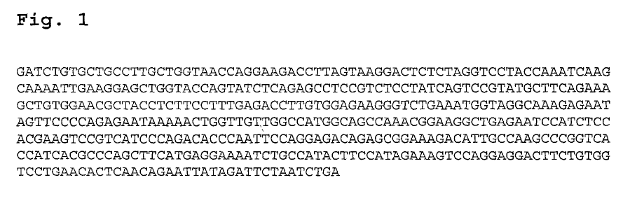

Suppression subtractive hybridization (SSH) (Diatchenko et al., 1996, PNAS 93:6025) was then used to identify novel genes, such as those that are overexpressed in prostate cancer, by comparing cDNAs from various androgen dependent and androgen independent LAPC xenografts. This strategy resulted in the identification of novel genes. One of these genes, designated 83P5G4, was identified from a subtraction where cDNA derived from an LAPC-4 AD tumor, 3 days post-castration, was subtracted from cDNA derived from an LAPC-4 AD tumor grown in an intact male. The SSH DNA sequence of about 445 b.p. (FIG. 1) is novel and exhibits homology only to expressed sequence tags (ESTs) in the dbEST database.

The 83P5G4 gene isolated using the SSH sequence as a probe encodes a putative nuclear protein that is up-regulated in prostate cancer. The expression of 83P5G4 in prostate cancer provides evidence that this protein has a functional role in tumor progression. It is possible that 83P5G4 functions as a transcription factor involved in activating genes involved in tumorigenesis or repressing genes that block tumorigenesis.

As is further described in the Examples that follow, the 83P5G4 gene and protein have been characterized using a number of analytical approaches. For example, analyses of nucleotide coding and amino acid sequences were conducted in order to identify potentially related molecules, as well as recognizable structural domains, topological features, and other elements within the 83P5G4 mRNA and protein structures. Northern blot analyses of 83P5G4 mRNA expression were conducted in order to establish the range of normal and cancerous tissues expressing 83P5G4 message.

A cDNA (clone 1) of 2838 b.p. was isolated from an LAPC-4 AD library, revealing an open reading frame (ORF) of 730 amino acids, with the codon for the N-terminal methionine occurring at nucleotides 130-132 as shown in FIG. 2. Alternatively, the codon for the N-terminal methionine of the open reading frame may occur at nucleotides 316-318 as shown in FIG. 2, thereby encoding a protein of 668 amino acids. The protein sequence reveals a single nuclear localization signal and is predicted to be nuclear in localization using the PSORT program (http://psort.nibb.ac.jp:8800/form.html; http://www.cbs.dtu.dk/). Sequence analysis of 83P5G4 reveals homology to the lethal (2) denticless protein of Drosophila (Kurzik-Dumke et al., 1996, Gene 171:163-170). The two protein sequences are 42% identical and 60% homologous over a 352 amino acid region (FIG. 3). The 83P5G4 amino acid sequence contains 5 predicted WD40 repeat domains, a nuclear localization signal (residues 199-203), two ser/pro rich regions (44% of amino acids within residues 425 and 520 and 43% of amino acids within residues 608-642), and a leucine zipper domain (residues 577-598).

As noted above, 83P5G4 represents a novel WD40 repeat protein that is highly expressed in prostate cancer. WD40 repeats were first identified in the beta-subunit of trimeric G proteins (Fong et al., 1986, PNAS 83:2162). There are currently about 30 known WD40 repeat containing proteins (Neer et al., 1994, Nature 371, 297-300). The WD40 regions are involved in protein-protein interactions between proteins involved in intracellular signaling. All WD40 proteins seem to be regulatory molecules involved in regulating processes such as cell division, cell-fate determination, gene transcription, transmembrane signaling, mRNA modification and vesicle fusion (Neer et al., 1994, Nature 371, 297-300). The closest homologue to 83P5G4, lethal (2) denticless (L2DT), is induced by heat shock and is involved in Drosophila development (Kurzik-Dumke et al., 1996, Gene 171:163-170). The WD repeat and leucine zipper domains indicate that 83P5G4 is likely to function as a regulatory protein that may be capable of interacting with other signaling proteins in signaling and/or transcriptional pathways. Its up-regulation in prostate cancer suggests a functional role in cancer pathobiology. Therefore, 83P5G4 has potential as a target for small molecule therapeutics. Investigating 83P5G4 function may also lead to identification of other potential targets.

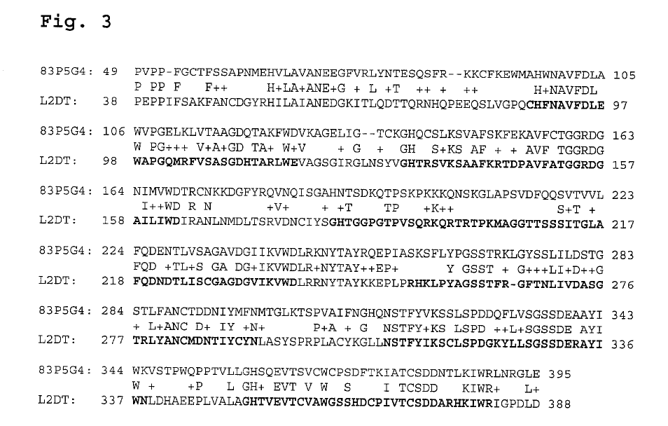

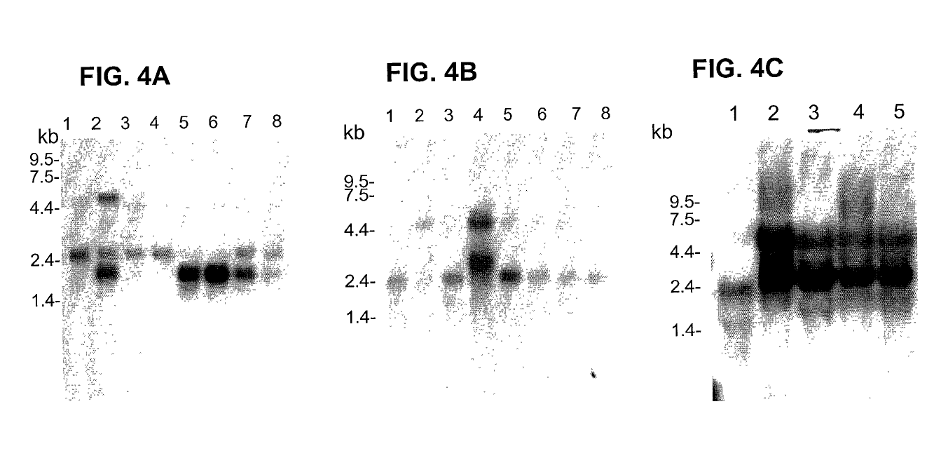

Northern blot analysis using an 83P5G4 SSH fragment probe performed on 16 normal tissues showed expression in all normal tissues tested (FIG. 4). The 83P5G4 gene produces three transcripts of 1.8, 2.5 and 4.5 kb. Different tissues express different transcripts. Brain is the only tissue that expresses all three transcripts. Liver, skeletal muscle, spleen, prostate and leukocytes only express the 1.8 kb transcript. Lung only expresses the 2.5 kb transcript. Kidney and pancreas express the 1.8 and 2.5 kb transcripts. Thymus, ovary, small intestine and colon express the 1.8 and 4.5 kb transcripts. Heart, placenta and testis express the 2.5 and 4.5 kb transcripts. The highest expression levels in normal tissues are detected in testis. The predominant bands in prostate cancer cells are the 2.5 and 4.5 kb bands.

To analyze 83P5G4 expression in prostate cancer tissues Northern blotting was performed on RNA derived from the LAPC xenografts. The results show very high expression levels of the 2.5 and 4.5 kb transcripts in LAPC-4 AD, LAPC-4 Al, LAPC-9 AD, and LAPC-9 AI. While it is unclear whether the different transcripts represent alternatively spliced isoform, or whether they represent unprocessed RNA species, the fact that different tissues express different transcripts suggest that the former is the case. It is possible that 83P5G4 isoforms expressed in the prostate cancer xenografts are the same isoforms that are expressed in testis. The results from the LAPC xenografts provide evidence that 83P5G4 is up-regulated in prostate cancer.

Properties of 83P5G4.

As disclosed herein, 83P5G4 exhibits specific properties that are analogous to those found in a family of molecules whose polynucleotides, polypeptides, reactive cytotoxic T cells (CTL), reactive helper T cells (HTL) and anti-polypeptide antibodies are used in well known diagnostic assays that examine conditions associated with disregulated cell growth such as cancer, in particular prostate cancer (see, e.g., both its highly specific pattern of tissue expression as well as its overexpression in prostate cancers as described for example in Example 3). The best-known member of this class is PSA, the archetypal marker that has been used by medical practitioners for years to identify and monitor the presence of prostate cancer (see, e.g., Merrill et al., J. Urol. 163(2):503-5120 (2000); Polascik et al., J. Urol. Aug; 162(2): 293-306 (1999) and Fortier et al., J. Nat. Cancer Inst. 91(19):1635-1640(1999)). A variety of other diagnostic markers are also used in this context including p53 and K-ras (see, e.g., Tulchinsky et al., Int J Mol Med 1999 Jul;4(1):99-102 and Minimoto et al., Cancer Detect Prev 2000;24(1):1-12). Therefore, this disclosure of the 83P5G4 polynucleotides and polypeptides (as well as the 83P5G4 polynucleotide probes and anti-83P5G4 antibodies used to identify the presence of these molecules) and their properties allows skilled artisans to utilize these molecules in methods that are analogous to those used, for example, in a variety of diagnostic assays directed to examining conditions associated with cancer.

Typical embodiments of diagnostic methods that utilize the 83P5G4 polynucleotides, polypeptides, reactive T cells and antibodies described herein are analogous to those methods from well-established diagnostic assays that employ PSA polynucleotides, polypeptides, reactive T cells and antibodies. For example, just as PSA polynucleotides are used as probes (for example in Northern analysis, see, e.g., Sharief et al., Biochem. Mol. Biol. Int. 33(3):567-74(1994)) and primers (for example in PCR analysis, see, e.g., Okegawa et al., J. Urol. 163(4): 1189-1190 (2000)) to observe the presence and/or the level of PSA mRNAs in methods of monitoring PSA overexpression or the metastasis of prostate cancers, the 83P5G4 polynucleotides described herein can be utilized in the same way to detect 83P5G4 overexpression or the metastasis of prostate and other cancers expressing this gene. Alternatively, just as PSA polypeptides are used to generate antibodies specific for PSA which can then be used to observe the presence and/or the level of PSA proteins in methods to monitor PSA protein overexpression (see, e.g., Stephan et al., Urology 55(4):560-3 (2000)) or the metastasis of prostate cells (see, e.g., Alanen et al., Pathol. Res. Pract. 192(3):233-7 (1996)), the 83P5G4 polypeptides described herein can be utilized to generate antibodies for use in detecting 83P5G4 overexpression or the metastasis of prostate cells and cells of other cancers expressing 83P5G4.

Specifically, because metastases involves the movement of cancer cells from an organ of origin (such as the lung or prostate gland etc.) to a different area of the body (such as a lymph node), assays which examine a biological sample for the presence of cells expressing 83P5G4 polynucleotides and/or polypeptides can be used to provide evidence of metastasis. For example, when a biological sample from tissue that does not normally contain 83P5G4-expressing cells (or contains cells that express specific isoforms of 83P5G4 mRNAs) is found to contain 83P5G4-expressing cells (or cells that express different isoforms of 83P5G4 mRNAs) such as the 83P5G4 expression seen in LAPC4 and LAPC9 xenografts isolated from lymph node and bone metastasis, respectively, this finding is indicative of metastasis.

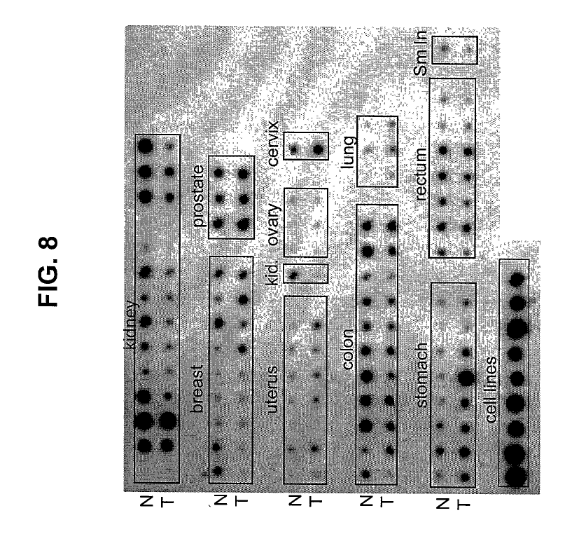

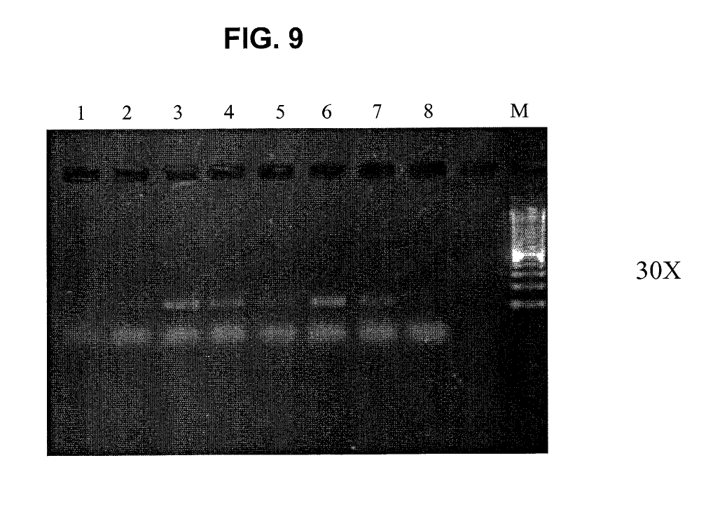

Alternatively 83P5G4 polynucleotides and/or polypeptides can be used to provide evidence of cancer, for example, when a cells in biological sample that do not normally express 83P5G4 or express 83P5G4 at a different level are found to express 83P5G4 or have an increased expression of 83P5G4 (see, e.g., the 83P5G4 expression in kidney, lung and colon cancer cells and in patient samples etc. shown in FIGS. 4-9). In such assays, artisans may further wish to generate supplementary evidence of metastasis by testing the biological sample for the presence of a second tissue restricted marker (in addition to 83P5G4) such as PSA, PSCA etc. (see, e.g., Alanen et al., Pathol. Res. Pract. 192(3):233-237 (1996)).

Just as PSA polynucleotide fragments and polynucleotide variants are employed by skilled artisans for use in methods of monitoring PSA, 83P5G4 polynucleotide fragments and polynucleotide variants are used in an analogous manner. In particular, typical PSA polynucleotides used in methods of monitoring PSA are probes or primers that consist of fragments of the PSA cDNA sequence. Illustrating this, primers used to PCR amplify a PSA polynucleotide must include less than the whole PSA sequence to function in the polymerase chain reaction. In the context of such PCR reactions, skilled artisans generally create a variety of different polynucleotide fragments that can be used as primers in order to amplify different portions of a polynucleotide of interest or to optimize amplification reactions (see, e.g., Caetano-Anolles, G. Biotechniques 25(3):472-476, 478-480 (1998); Robertson et al., Methods Mol. Biol. 98:121-154 (1998)). An additional illustration of the use of such fragments is provided in Example 3, where an 83P5G4 polynucleotide fragment is used as a probe to show the overexpression of 83P5G4 mRNAs in cancer cells. In addition, variant polynucleotide sequences are typically used as primers and probes for the corresponding mRNAs in PCR and Northern analyses (see, e.g., Sawai et al., Fetal Diagn. Ther. November-December 1996; 11(6):407-13 and Current Protocols In Molecular Biology, Volume 2, Unit 2, Frederick M. Ausubul et al. eds., 1995)). Polynucleotide fragments and variants are useful in this context where they are capable of binding to a target polynucleotide sequence (e.g. the 83P5G4 polynucleotide shown in SEQ ID NO: 1) under conditions of high stringency.

Just as PSA polypeptide fragments and polypeptide variants are employed by skilled artisans for use in methods of monitoring the PSA molecule, 83P5G4 polypeptide fragments and polypeptide analogs or variants can also be used in an analogous manner. In particular, typical PSA polypeptides used in methods of monitoring PSA are fragments of the PSA protein that contain an epitope that can be recognized by an antibody or T cell that specifically binds to that epitope. This practice of using polypeptide fragments or polypeptide variants to generate antibodies (such as anti-PSA antibodies or T cells) is typical in the art with a wide variety of systems such as fusion proteins being used by practitioners (see, e.g., Current Protocols In Molecular Biology, Volume 2, Unit 16, Frederick M. Ausubul et al. eds., 1995). In this context, each epitope(s) functions to provide the architecture with which an antibody or T cell is reactive. Typically, skilled artisans generally create a variety of different polypeptide fragments that can be used in order to generate antibodies specific for different portions of a polypeptide of interest (see, e.g., U.S. Pat. No. 5,840,501 and U.S. Pat. No. 5,939,533). For example it may be preferable to utilize a polypeptide comprising one of the 83P5G4 biological motifs discussed herein or available in the art (see, e.g., http://www.ebi.ac.uk/interpro/scan.html). Polypeptide fragments, variants or analogs are typically useful in this context as long as they comprise an epitope capable of generating an antibody or T cell specific for a target polypeptide sequence (e.g. the 83P5G4 polypeptide shown in SEQ ID NO: 2).

As shown herein, the 83P5G4 polynucleotides and polypeptides (as well as the 83P5G4 polynucleotide probes and anti-83P5G4 antibodies or T cells used to identify the presence of these molecules) exhibit specific properties that make them useful in diagnosing cancers of the prostate. Diagnostic assays that measure the presence of 83P5G4 gene products, in order to evaluate the presence or onset of a disease condition described herein, such as prostate cancer, are used to identify patients for preventive measures or further monitoring, as has been done so successfully with PSA. Moreover, these materials satisfy a need in the art for molecules having similar or complementary characteristics to PSA in situations where, for example, a definite diagnosis of metastasis of prostatic origin cannot be made on the basis of a test for PSA alone (see, e.g., Alanen et al., Pathol. Res. Pract. 192(3):233-237 (1996)), and consequently, materials such as 83P5G4 polynucleotides and polypeptides (as well as the 83P5G4 polynucleotide probes and anti-83P5G4 antibodies used to identify the presence of these molecules) must be employed to confirm metastases of prostatic origin.

Finally, in addition to their use in diagnostic assays, the 83P5G4 polynucleotides disclosed herein have a number of other specific utilities such as their use in the identification of oncogenetic associated chromosomal abnormalities in 1q31-1q32.1, the chromosomal region to which the 83P5G4 gene maps (see Example 7 below). Moreover, in addition to their use in diagnostic assays, the 83P5G4-related proteins and polynucleotides disclosed herein have other utilities such as their use in the forensic analysis of tissues of unknown origin (see, e.g., Takahama K Forensic Sci Int Jun. 28, 1996; 80(1-2): 63-9).

### 83P5G4 POLYNUCLEOTIDES

One aspect of the invention provides polynucleotides corresponding or complementary to all or part of an 83P5G4 gene, mRNA, and/or coding sequence, preferably in isolated form, including polynucleotides encoding an 83P5G4-related protein and fragments thereof, DNA, RNA, DNA/RNA hybrid, and related molecules, polynucleotides or oligonucleotides complementary to an 83P5G4 gene or mRNA sequence or a part thereof, and polynucleotides or oligonucleotides that hybridize to an 83P5G4 gene, mRNA, or to an 83P5G4 encoding polynucleotide (collectively, “83P5G4 polynucleotides”).

One embodiment of an 83P5G4 polynucleotide, and any protein encoded thereby, is an 83P5G4 polynucleotide having the sequence shown in SEQ ID NO: 1. A 83P5G4 polynucleotide can comprise a polynucleotide having the nucleotide sequence of human 83P5G4 as shown in SEQ ID NO: 1, wherein T can also be U; a polynucleotide that encodes all or part of the 83P5G4 protein; a sequence complementary to the foregoing; or a polynucleotide fragment of any of the foregoing. Another embodiment comprises a polynucleotide encoding an 83P5G4 polypeptide whose sequence is encoded by the cDNA contained in the plasmid as deposited with American Type Culture Collection as Accession No. PTA-1 154. Another embodiment comprises a polynucleotide, and any peptide encoded thereby, that is capable of hybridizing under stringent hybridization conditions to the human 83P5G4 cDNA shown in SEQ ID NO: 1 or to a polynucleotide fragment thereof. Another embodiment comprises a polynucleotide, and any peptide encoded thereby, that is:

(a) a polynucleotide having the sequence as shown in SEQ ID NO: 1 from nucleotide residue number 1 through nucleotide residue number 879 of SEQ ID NO: 1; or,

(b) a polynucleotide having the sequence as shown in SEQ ID NO: 1 from nucleotide residue number 130 through nucleotide residue number 879 of SEQ ID NO: 1; or,

(c) a polynucleotide having the sequence as shown in SEQ ID NO: 1 from nucleotide residue number 2134 through nucleotide residue number 2838 of SEQ ID NO: 1; or,

(d) a polynucleotide having the sequence as shown in SEQ ID NO: 1 from nucleotide residue number 2134 through nucleotide residue number 2322 of SEQ ID NO: 1; or,

(e) a polynucleotide whose starting base is in a range of 1-879 of FIG. 2 (SEQ ID NO: 1) and whose ending base is in a range of 880-2838 of FIG. 2 (SEQ ID NO: 1); or,

(f) a polynucleotide whose starting base is in a range of 130-879 of FIG. 2 (SEQ ID NO: 1) and whose ending base is in a range of 880-2322 of FIG. 2 (SEQ ID NO: 1); or,

(g) a polynucleotide whose starting base is in a range of 880-2133 of FIG. 2 (SEQ ID NO: 1) and whose ending base is in a range of 2134-2838 of FIG. 2 (SEQ ID NO: 1); or,

(h) a polynucleotide whose starting base is in a range of 880-2133 of FIG. 2 (SEQ ID NO: 1) and whose ending base is in a range of 2134-2322 of FIG. 2 (SEQ ID NO: 1); or,

(i) a polynucleotide whose starting base is in a range of 130-879 of FIG. 2 (SEQ ID NO: 1) and whose ending base is in a range of 2134-2322 of FIG. 2 (SEQ ID NO: 1); or,

(j) a polynucleotide of (a)-(i) that is more than 10 nucleotide bases in length; or

a polynucleotide that selectively hybridizes under stringent conditions to a polynucleotide of (a)-(j);

where a range is understood to specifically disclose each whole unit position thereof.

Also within the scope of the invention is a nucleotide, as well as any peptide encoded thereby, that starts at any of the following positions and ends at a higher position: 1, a range of bases 1-879, 879, 880, a range of bases 880-2133, 2133, 2134, a range of bases 2134-2838, and 2838; wherein a range as used in this section is understood to specifically disclose all whole unit positions thereof, i.e. each particular base number.

Typical embodiments of the invention disclosed herein include 83P5G4 polynucleotides that encode specific portions of the 83P5G4 mRNA sequence (and those which are complementary to such sequences) such as those that encode the protein and fragments thereof, for example of 4, 5, 6, 7, 8, 9, 10, 11, 12, 13, 14, 15 or more contiguous amino acids. For example, representative embodiments of the invention disclosed herein include: polynucleotides and their encoded peptides themselves encoding about amino acid 1 to about amino acid 10 of the 83P5G4 protein shown in FIG. 2 (SEQ ID NO: 2), polynucleotides encoding about amino acid 10 to about amino acid 20 of the 83P5G4 protein shown in FIG. 2, polynucleotides encoding about amino acid 20 to about amino acid 30 of the 83P5G4 protein shown in FIG. 2, polynucleotides encoding about amino acid 30 to about amino acid 40 of the 83P5G4 protein shown in FIG. 2 , polynucleotides encoding about amino acid 40 to about amino acid 50 of the 83P5 G4 protein shown in FIG. 2 , polynucleotides encoding about amino acid 50 to about amino acid 60 of the 83P5G4 protein shown in FIG. 2, polynucleotides encoding about amino acid 60 to about amino acid 70 of the 83P5G4 protein shown in FIG. 2, polynucleotides encoding about amino acid 70 to about amino acid 80 of the 83P5G4 protein shown in FIG. 2, polynucleotides encoding about amino acid 80 to about amino acid 90 of the 83P5G4 protein shown in FIG. 2 and polynucleotides encoding about amino acid 90 to about amino acid 100 of the 83P5G4 protein shown in FIG. 2, in increments of about 10 amino acids, ending at amino acid 730. Accordingly polynucleotides encoding portions of the amino acid sequence (of about 10 amino acids), of amino acids 100-730 of the 83P5G4 protein are embodiments of the invention.

Polynucleotides encoding larger portions of the 83P5G4 protein are also contemplated. For example polynucleotides encoding from about amino acid 1 (or 20 or 30 or 40 etc.) to about amino acid 20, (or 30, or 40 or 50 etc.) of the 83P5G4 protein shown in FIG. 2 can be generated by a variety of techniques well known in the art. These polynucleotide fragments can include any portion of the 83P5G4 sequence as shown in FIG. 2, for example a polynucleotide having the sequence as shown in FIG. 2 from nucleotide residue number 132 through nucleotide residue number 2324.

Additional illustrative embodiments of the invention disclosed herein include 83P5G4 polynucleotide fragments encoding one or more of the biological motifs contained within the 83P5G4 protein sequence. In one embodiment, typical polynucleotide fragments of the invention can encode one or more of the nuclear localization sequences or disclosed herein. In another embodiment, typical polynucleotide fragments of the invention can encode one or more of the region of 83P5G4 that exhibits homology to the lethal (2) denticless protein of Drosophila, a WD repeat domain or a ser/pro rich region. In another embodiment of the invention, typical polynucleotide fragments can encode one or more of the 83P5G4 N-glycosylation sites, cAMP and cCMP-dependent protein kinase phosphorylation sites, casein kinase II phosphorylation sites or N-myristoylation sites as disclosed in greater detail in the text discussing the 83P5G4 protein and polypeptides below. The embodiments of the invention which consist of polypeptides containing specific biological motifs of the 83P5G4 protein encoded by the polynucleotides discussed above are discussed in greater detail in the text discussing the 83P5G4 protein and polypeptides herein. In yet another embodiment of the invention, typical polynucleotide fragments can comprise sequences that are common or unique to one or more 83P5G4 alternative splicing variants, such as the splice variants that generate either the 1.8 or the 2.5 or the 4.5 KB transcripts that are overexpressed in prostate cancers shown for example in FIG. 4.

The polynucleotides of the preceding paragraphs have a number of different specific uses. For example, because the human 83P5G4 gene maps to chromosome 1q3 1-q32.1, polynucleotides encoding different regions of the 83P5G4 protein can be used to characterize cytogenetic abnormalities on chromosome 1, bands q31 and q32, that have been identified as being associated with various cancers. In particular, a variety of chromosomal abnormalities in 1q31-q32.1 including rearrangements have been identified as frequent cytogenetic abnormalities in a number of different cancers (see e.g. Forozan et al., Cancer Res. 60(16):4519-4525 (2000); Benitez et al., Cancer Res. 57(19):4217-4220 (1997); and Kallioniemi et al., Genes Chromosomes Cancer 12(3):213-219 (1995)). Consequently, polynucleotides encoding specific regions of the 83P5G4 protein provide new tools that can be used to delineate with a greater precision than previously possible, the specific nature of the cytogenetic abnormalities in this region of chromosome 1 that may contribute to the malignant phenotype. In this context, these polynucleotides satisfy a need in the art for expanding the sensitivity of chromosomal screening in order to identify more subtle and less common chromosomal abnormalities (see e.g. Evans et al., Am. J. Obstet. Gynecol 171(4):1055-1057 (1994)).

Alternatively, as 83P5G4 was shown to be highly expressed in prostate and other cancers (FIGS. 4-9), 83P5G4 polynucleotides are used in methods assessing the status of 83P5G4 gene products in normal versus cancerous tissues. Typically, polynucleotides that encode specific regions of the 83P5G4 protein are used to assess the presence of perturbations (such as deletions, insertions, point mutations, or alterations resulting in a loss of an antigen etc.) in specific regions of the 83P5G4 gene products, such as such regions containing a nuclear localization signal. Exemplary assays include both RT-PCR assays as well as single-strand conformation polymorphism (SSCP) analysis (see, e.g., Marrogi et al., J. Cutan. Pathol. 26(8):369-378 (1999), both of which utilize polynucleotides encoding specific regions of a protein to examine these regions within the protein.

Other specifically contemplated nucleic acid related embodiments of the invention disclosed herein are genomic DNA, cDNAs, ribozymes, and antisense molecules, as well as nucleic acid molecules based on an alternative backbone or including alternative bases, whether derived from natural sources or synthesized. For example, antisense molecules can be RNAs or other molecules, including peptide nucleic acids (PNAs) or non-nucleic acid molecules such as phosphorothioate derivatives that specifically bind DNA or RNA in a base pair-dependent manner. A skilled artisan can readily obtain these classes of nucleic acid molecules using the 83P5G4 polynucleotides and polynucleotide sequences disclosed herein.

Antisense technology entails the administration of exogenous oligonucleotides that bind to a target polynucleotide located within the cells. The term “antisense” refers to the fact that such oligonucleotides are complementary to their intracellular targets, e.g., 83P5G4. See for example, Jack Cohen, Oligodeoxynucleotides, Antisense Inhibitors of Gene Expression, CRC Press, 1989; and Synthesis 1:1-5 (1988). The 83P5G4 antisense oligonucleotides of the present invention include derivatives such as S-oligonucleotides (phosphorothioate derivatives or S-oligos, see, Jack Cohen, supra), which exhibit enhanced cancer cell growth inhibitory action. S-oligos (nucleoside phosphorothioates) are isoelectronic analogs of an oligonucleotide (O-oligo) in which a nonbridging oxygen atom of the phosphate group is replaced by a sulfur atom. The S-oligos of the present invention can be prepared by treatment of the corresponding O-oligos with 3H-1,2-benzodithiol-3-one-1,1-dioxide, which is a sulfur transfer reagent. See Iyer, R. P. et al, J. Org. Chem. 55:4693-4698 (1990); and Iyer, R. P. et al., J. Am. Chem Soc. 112:1253-1254 (1990). Additional 83P5G4 antisense oligonucleotides of the present invention include morpholino antisense oligonucleotides known in the art (see, e.g., Partridge et al., 1996, Antisense & Nucleic Acid Drug Development 6:169-175).

The 83P5G4 antisense oligonucleotides of the present invention typically can be RNA or DNA that is complementary to and stably hybridizes with the first 100 5′ codons or last 100 3′ codons of the 83P5G4 genomic sequence or the corresponding mRNA. Absolute complementarity is not required, although high degrees of complementarity are preferred. Use of an oligonucleotide complementary to this region allows for the selective hybridization to 83P5G4 mRNA and not to mRNA specifying other regulatory subunits of protein kinase. In one embodiment, 83P5G4 antisense oligonucleotides of the present invention are 15 to 30-mer fragments of the antisense DNA molecule that have a sequence that hybridizes to 83P5G4 mRNA. Optionally, 83P5G4 antisense oligonucleotide is a 30-mer oligonucleotide that is complementary to a region in the first 10 5′ codons or last 10 3′ codons of 83P5G4. Alternatively, the antisense molecules are modified to employ ribozymes in the inhibition of 83P5G4 expression, see, e.g., L. A. Couture & D. T. Stinchcomb; Trends Genet 12:510-515 (1996).

Further specific embodiments of this aspect of the invention include primers and primer pairs, which allow the specific amplification of polynucleotides of the invention or of any specific parts thereof, and probes that selectively or specifically hybridize to nucleic acid molecules of the invention or to any part thereof. Probes can be labeled with a detectable marker, such as, for example, a radioisotope, fluorescent compound, bioluminescent compound, a chemiluminescent compound, metal chelator or enzyme. Such probes and primers are used to detect the presence of an 83P5G4 polynucleotide in a sample and as a means for detecting a cell expressing an 83P5G4 protein.

Examples of such probes include polypeptides comprising all or part of the human 83P5G4 cDNA sequences shown in FIG. 2. Examples of primer pairs capable of specifically amplifying 83P5G4 mRNAs are also described in the Examples that follow. As will be understood by the skilled artisan, a great many different primers and probes can be prepared based on the sequences provided herein and used effectively to amplify and/or detect an 83P5G4 mRNA.

The 83P5G4 polynucleotides of the invention are useful for a variety of purposes, including but not limited to their use as probes and primers for the amplification and/or detection of the 83P5G4 gene(s), mRNA(s), or fragments thereof; as reagents for the diagnosis and/or prognosis of prostate cancer and other cancers; as coding sequences capable of directing the expression of 83P5G4 polypeptides; as tools for modulating or inhibiting the expression of the 83P5G4 gene(s) and/or translation of the 83P5G4 transcript(s); and as therapeutic agents.

### ISOLATION OF 83P5G4-ENCODING NUCLEIC ACID MOLECULES

The 83P5G4 cDNA sequences described herein enable the isolation of other polynucleotides encoding 83P5G4 gene product(s), as well as the isolation of polynucleotides encoding 83P5G4 gene product homologs, alternatively spliced isoforms, allelic variants, and mutant forms of the 83P5G4 gene product as well as polynucleotides that encode analogs of 83P5G4-related proteins. Various molecular cloning methods that can be employed to isolate full-length cDNAs encoding an 83P5G4 gene are well-known (See, for example, Sambrook, J. et al., Molecular Cloning: A Laboratory Manual, 2d edition., Cold Spring Harbor Press, New York, 1989; Current Protocols in Molecular Biology. Ausubel et al., Eds., Wiley and Sons, 1995). For example, lambda phage cloning methodologies can be conveniently employed, using commercially available cloning systems (e.g., Lambda ZAP Express, Stratagene). Phage clones containing 83P5G4 gene cDNAs can be identified by probing with a labeled 83P5G4 cDNA or a fragment thereof. For example, in one embodiment, the 83P5G4 cDNA (FIG. 2) or a portion thereof can be synthesized and used as a probe to retrieve overlapping and full-length cDNAs corresponding to an 83P5G4 gene. The 83P5G4 gene itself can be isolated by screening genomic DNA libraries, bacterial artificial chromosome libraries (BACs), yeast artificial chromosome libraries (YACs), and the like, with 83P5G4 DNA probes or primers.

### RECOMBINANT DNA MOLECULES AND HOST-VECTOR SYSTEMS

The invention also provides recombinant DNA or RNA molecules containing a 83P5G4 polynucleotide or a fragment or analog or homologue thereof, including but not limited to phages, plasmids, phagemids, cosmids, YACs, BACs, as well as various viral and non-viral vectors well-known in the art, and cells transformed or transfected with such recombinant DNA or RNA molecules. Methods for generating such molecules are well-known (see, for example, Sambrook et al, 1989, supra).

The invention further provides a host-vector system comprising a recombinant DNA molecule containing an 83P5G4 polynucleotide, fragment, analog or homologue thereof within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9 or HighFive cell). Examples of suitable mammalian cells include various prostate cancer cell lines such as DU145 and TsuPr1, other transfectable or transducible prostate cancer cell lines, primary cells (PrEC), as well as a number of mammalian cells routinely used for the expression of recombinant proteins (e.g., COS, CHO, 293, 293T cells). More particularly, a polynucleotide comprising the coding sequence of 83P5G4 or a fragment, analog or homolog thereof can be used to generate 83P5G4 proteins or fragments thereof using any number of host-vector systems routinely used and widely known in the art.

A wide range of host-vector systems suitable for the expression of 83P5G4 proteins or fragments thereof are available, see for example, Sambrook et al., 1989, supra; Current Protocols in Molecular Biology, 1995, supra). Preferred vectors for mammalian expression include but are not limited to pcDNA 3.1 myc-His-tag (Invitrogen) and the retroviral vector pSRαtkneo (Muller et al., 1991, MCB 11:1785). Using these expression vectors, 83P5G4 can be expressed in several prostate cancer and non-prostate cell lines, including for example 293, 293T, rat-1,NIH 3T3 and TsuPr1. The host-vector systems of the invention are useful for the production of a 83P5G4 protein or fragment thereof. Such host-vector systems can be employed to study the functional properties of 83P5G4 and 83P5G4 mutations or analogs.

Recombinant human 83P5G4 protein or an analog or homolog or fragment thereof can be produced by mammalian cells transfected with a construct encoding 83P5G4. In an illustrative embodiment described in the Examples, 293T cells can be transfected with an expression plasmid encoding 83P5G4 or fragment, analog or homolog thereof, the 83P5G4 or related protein is expressed in the 293T cells, and the recombinant 83P5G4 protein is isolated using standard purification methods (e.g., affinity purification using anti-83P5G4 antibodies). In another embodiment, also described in the Examples herein, the 83P5G4 coding sequence is subcloned into the retroviral vector pSRαMSVtkneo and used to infect various mammalian cell lines, such as NIH 3T3, TsuPr1, 293 and rat-1 in order to establish 83P5G4-expressing cell lines. Various other expression systems well-known in the art can also be employed. Expression constructs encoding a leader peptide joined in frame to the 83P5G4 coding sequence can be used for the generation of a secreted form of recombinant 83P5G4 protein.

Proteins encoded by the 83P5G4 genes, or by analogs, homologs or fragments thereof, have a variety of uses, including but not limited to generating antibodies and in methods for identifying ligands and other agents and cellular constituents that bind to an 83P5G4 gene product. Antibodies raised against a 83P5G4 protein or fragment thereof are useful in diagnostic and prognostic assays, and imaging methodologies in the management of human cancers characterized by expression of 83P5G4 protein, including but not limited to cancers of the prostate, bladder, kidney, brain, bone, cervix, uterus, ovary, breast, pancreas, stomach, colon, rectal, leukocytes and lung. Such antibodies can be expressed intracellularly and used in methods of treating patients with such cancers. 83P5G4-related nucleic acids or proteins are also used in generating HTL or CTL responses.

Various immunological assays useful for the detection of 83P5G4 proteins are contemplated, including but not limited to various types of radioimmunoassays, enzyme-linked immunosorbent assays (ELISA), enzyme-linked imnmunofluorescent assays (ELIFA), inmmunocytochemical methods, and the like. Antibodies can be labeled and used as immunological imaging reagents capable of detecting 83P5G4-expressing cells (e.g., in radioscintigraphic imaging methods). 83P5G4 proteins are also particularly useful in generating cancer vaccines, as further described herein.

### 83P5G4-RELATED PROTEINS

Another aspect of the present invention provides 83P5G4-related proteins and polypeptide fragments thereof. Specific embodiments of 83P5G4 proteins comprise a polypeptide having all or part of the amino acid sequence of human 83P5G4 as shown in FIG. 2. Alternatively, embodiments of 83P5G4 proteins comprise variant or analog polypeptides that have alterations in the amino acid sequence of 83P5G4 shown in FIG. 2.

In general, naturally occurring allelic variants of human 83P5G4 share a high degree of structural identity and homology (e.g., 90% or more identity). Typically, allelic variants of the 83P5G4-related proteins contain conservative amino acid substitutions within the 83P5G4 sequences described herein or contain a substitution of an amino acid from a corresponding position in a homologue of 83P5G4. One class of 83P5G4 allelic variants are proteins that share a high degree of homology with at least a small region of a particular 83P5G4 amino acid sequence, but further contain a radical departure from the sequence, such as a non-conservative substitution, truncation, insertion or frame shift. In comparisons of protein sequences, the terms, similarity, identity, and homology each have a distinct meaning in the field of genetics.

Amino acid abbreviations are provided in Table IIA. Conservative amino acid substitutions can frequently be made in a protein without altering either the conformation or the function of the protein. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table IIB herein; pages 13-15 “Biochemistry” 2^{nd} ED. Lubert Stryer ed (Stanford University); Henikoff et al., PNAS 1992 Vol 89 10915-10919; Lei et al., J Biol Chem May 19, 1995; 270(20): 11882-6).

Embodiments of the invention disclosed herein include a wide variety of art accepted variants or analogs of 83P5G4 proteins such as polypeptides having amino acid insertions, deletions and substitutions. 83P5G4 variants can be made using methods known in the art such as site-directed mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the 83P5G4 variant DNA.

Scanning amino acid analysis can also be employed to identify one or more amino acids along a contiguous sequence that is involved in a specific biological activity such as a protein-protein interaction. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions [Creighton, The Proteins, (W. H. Freeman & Co., N.Y.); Chothia, J. Mol. Biol., 150:1 (1976)]. If alanine substitution does not yield adequate amounts of variant, an isosteric amino acid can be used.

As defined herein, 83P5G4 variants, analogs or homologs, have the distinguishing attribute of having at least one epitope “in common” with a 83P5G4 protein having the amino acid sequence of SEQ ID NO: 2. As used in this sentence, “in common” means such an antibody or T cell that specifically binds to an 83P5G4 variant also specifically binds to the 83P5G4 protein having the amino acid sequence of SEQ ID NO: 2. A polypeptide ceases to be a variant of the protein shown in SEQ ID NO: 2 when it no longer contains an epitope capable of being recognized by an antibody or T cell that specifically binds to a 83P5G4 protein. Those skilled in the art understand that antibodies that recognize proteins bind to epitopes of varying size, and a grouping of the order of about four or five amino acids, contiguous or not, is regarded as a typical number of amino acids in a minimal epitope. See, e.g., Nair et al., J. Immunol 2000 165(12): 6949-6955; Hebbes et al., Mol Immunol (1989) 26(9): 865-73; Schwartz et al., J Immunol (1985) 135(4): 2598-608. Another specific class of 83P5G4-related protein variants shares 70%, 75%, 80%, 85% or 90% or more similarity with the amino acid sequence of SEQ ID NO: 2 or a fragment thereof. Another specific class of 83P5G4 protein variants or analogs comprise one or more of the 83P5G4 biological motifs described herein or presently known in the art. Thus, encompassed by the present invention are analogs of 83P5G4 fragments (nucleic or amino acid) that have altered functional (e.g. immunogenic) properties relative to the starting fragment. It is to be appreciated that motifs now or which become part of the art are to be applied to the nucleic or amino acid sequences of FIG. 2.

As discussed herein, embodiments of the claimed invention include polypeptides containing less than the 730 amino acid sequence of the 83P5G4 protein shown in FIG. 2. For example, representative embodiments of the invention comprise peptides/proteins having any 4, 5, 6, 7, 8, 9, 10, 11, 12, 13, 14, 15 or more contiguous amino acids of the 83P5G4 protein shown in FIG. 2 (SEQ ID NO: 2). Moreover, representative embodiments of the invention disclosed herein include polypeptides consisting of about amino acid 1 to about amino acid 10 of the 83P5G4 protein shown in FIG. 2, polypeptides consisting of about amino acid 10 to about amino acid 20 of the 83P5G4 protein shown in FIG. 2, polypeptides consisting of about amino acid 20 to about amino acid 30 of the 83P5G4 protein shown in FIG. 2, polypeptides consisting of about amino acid 30 to about amino acid 40 of the 83P5G4 protein shown in FIG. 2, polypeptides consisting of about amino acid 40 to about amino acid 50 of the 83P5G4 protein shown in FIG. 2, polypeptides consisting of about amino acid 50 to about amino acid 60 of the 83P5G4 protein shown in FIG. 2, polypeptides consisting of about amino acid 60 to about amino acid 70 of the 83P5G4 protein shown in FIG. 2, polypeptides consisting of about amino acid 70 to about amino acid 80 of the 83P5G4 protein shown in FIG. 2, polypeptides consisting of about amino acid 80 to about amino acid 90 of the 83P5G4 protein shown in FIG. 2 and polypeptides consisting of about amino acid 90 to about amino acid 100 of the 83P5G4 protein shown in FIG. 2, etc. throughout the entirety of the 83P5G4 sequence. Following this scheme, polypeptides consisting of portions of the amino acid sequence of amino acids 100-730 of the 83P5G4 protein are typical embodiments of the invention. Accordingly, polypeptides consisting of about amino acid 1 (or 20 or 30 or 40 etc.) to about amino acid 20, (or 30, or 40 or 50 etc.) of the 83P5G4 protein shown in FIG. 2 in increments of about 10 amino acids, ending at amino acid 730 are embodiments of the invention. It is to be appreciated that the starting and stopping positions in this paragraph refer to the specified position as well as that position plus or minus 5 residues.

Additional illustrative embodiments of the invention disclosed herein include 83P5G4 polypeptides containing the amino acid residues of one or more of the biological motifs contained within the 83P5G4 polypeptide sequence as shown in FIG. 2. In another embodiment, polypeptides of the invention comprise one or more of the 83P5G4 nuclear localization sequences such as KPKKK at amino acids 199-203 of SEQ ID NO: 2 and/or PSKPKKKQNS at amino acids 197-206 of SEQ ID NO: 2. In another embodiment, polypeptides of the invention comprise one or more of the 83P5G4 ser/pro rich regions (44% of amino acids within residues 425-520 of SEQ ID NO: 2, and 43% of amino acids within residues 608-642 of SEQ ID NO: 2). In another embodiment, polypeptides of the invention comprise one or more of the 83P5G4 N-glycosylation sites such as NTSD at residues 190-193 of SEQ ID NO: 2, NYTA at residues 248-251 of SEQ ID NO: 2, NCTD at residues 289-292 of SEQ ID NO: 2, NMTG at residues 299-302 of SEQ ID NO: 2 and/or NSTF at residues 316-319 of SEQ ID NO: 2. In another embodiment, polypeptides of the invention comprise one or more of the regions of 83P5G4 that exhibit homology to the lethal (2) denticless protein of Drosophila. In another embodiment, polypeptides of the invention comprise the regions of 83P5G4 that contain a leucine zipper pattern such as LDGQVENLHLDLCCLAGNQEDL at residues 577-598 of SEQ ID NO: 2. In another embodiment, polypeptides of the invention comprise one or more of the 83P5G4 cAMP and cGMP-dependent protein kinase phosphorylation sites such as KKES at residues 413-416 of SEQ ID NO: 2, RRGS at residues 482-485 of SEQ ID NO: 2 and/or RRQS at residues 688-691 of SEQ ID NO: 2. In another embodiment, polypeptides of the invention comprise one or more of the 83P5G4 Protein Kinase C phosphorylation sites such as SFR at residues 85-87 of SEQ ID NO: 2, TAK at residues 121-123 of SEQ ID NO: 2, TCK at residues 135-137 of SEQ ID NO: 2, SLK at residues 142-144 of SEQ ID NO: 2, SDK at residues 192-194 of SEQ ID NO: 2, STR at residues 268-270 of SEQ ID NO: 2, TRK at residues 269-271 of SEQ ID NO: 2, TLK at residues 384-386 of SEQ ID NO: 2, SQK at residues 410-412 of SEQ ID NO: 2, SQK at residues 535-537 of SEQ ID NO: 2, SIK at residues 468-470 of SEQ ID NO: 2, SPK at residues 490-492 of SEQ ID NO: 2, SFK at residues 496-498 of SEQ ID NO: 2, SIR at residues 500-502 of SEQ ID NO: 2, SPR at residues 526-528 of SEQ ID NO: 2, SPR at residues 676-678 of SEQ ID NO: 2, SVK at residues 562-564 of SEQ ID NO: 2, and/or SSK at residues 608-610 of SEQ ID NO: 2. In another embodiment, polypeptides of the invention comprise one or more of the 83P5G4 casein kinase II phosphorylation sites such as SGND at residues 35-38 of SEQ ID NO: 2, SYGE at residues 42-45 of SEQ ID NO: 2, SKFE at residues 149-152 of SEQ ID NO: 2, SPDD at residues 326-329 of SEQ ID NO: 2, SSDE at residues 336-339 of SEQ ID NO: 2, TCSD at residues 378-381 of SEQ ID NO: 2, SQAE at residues 539-542 of SEQ ID NO: 2, SCLE at residues 558-561 of SEQ ID NO: 2, TELD at residues 575-578 of SEQ ID NO: 2, SKIE at residues 609-612, SISE at residues 617-620, SSPE at residues 655-658 of SEQ ID NO: 2 and/or SQED at residues 717-720 of SEQ ID NO: 2. In another embodiment, polypeptides of the invention comprise one or more of the N-myristoylation sites such as GVLRNG at residues 13-18 of SEQ ID NO: 2, GCTFSS at residues 54-59 of SEQ ID NO: 2, GTCKGH at residues 134-139 of SEQ ID NO: 2, GGRDGN at residues 159-164 of SEQ ID NO: 2, GAHNTS at residues 187-192 of SEQ ID NO: 2, GLAPSV at residues 208-213 of SEQ ID NO: 2, GAVDGI at residues 234-239 of SEQ ID NO: 2, GSVSSV at residues 484-489 of SEQ ID NO: 2, GQVENL at residues 579-584 of SEQ ID NO: 2, GAGTSI at residues 613-618 of SEQ ID NO: 2 and/or GTSISE at residues 615-620 of SEQ ID NO: 2. In another embodiment, polypeptides of the invention comprise one or more of the CTF/NF-1 family sites at residues 669-701 of SEQ ID NO: 2 or residues 432-464 of SEQ ID NO: 2. In another embodiment, polypeptides of the invention comprise the nuclear transition protein 2 site at residues 617-642 of SEQ ID NO: 2. In another embodiment, polypeptides of the invention comprise one or more of the WD repeats such as AHWNAVFDLAWVPGELKLVTAAGDQTAKFWD at residues 96-126 of SEQ ID NO: 2, GHQCSLKSVAFSKFEKAVFCTGGRDGNIMVWD at residues 138-169 of SEQ ID NO: 2, AHNTSDKQTPSKPKKKQNSKGLAPSVDFQQSVTVVLFQDENTLVSAGAVDGIIKVWD at residues 188-244 of SEQ ID NO: 2, GHQNSTFYVKSSLSPDDQFLVSGSSDEAAYIWK at residues 313-345 of SEQ ID NO: 2 and/or GHSQEVTSVCWCPSDFTKIATCSDDNTLKIWR at residues 358-389 of SEQ ID NO: 2. Related embodiments of these inventions include polypeptides containing combinations of the different motifs discussed above with preferable embodiments being those that contain no insertions, deletions or substitutions either within the motifs or the intervening sequences of these polypeptides.

Illustrative examples of such embodiments includes a polypeptide having one or more amino acid sequences selected from the group consisting of NTSD, NYTA, NCTD, NMTG, NSTF, RRGS, SFR, TAK, TCK, SLK, SDK, STR, TRK, SQK, SPK, SFK, SIR, SPR, SGND, SYGE, SKFE, SQAE, GCTFSS, GTCKGH, GGRDGN, GAHNTS, GLAPSV, GAVDGI, GSVSSV, LVTAAGDQTAKFWDV and VSAGAVDGIIKVWDL of SEQ ID NO: 2 as noted above. In a preferred embodiments, the polypeptide includes two three or four or five or six or more amino acid sequences selected from the group consisting of NTSD, NYTA, NCTD, NMTG, NSTF, RRGS, SFR, TAK, TCK, SLK, SDK, STR, TRK, SQK, SPK, SFK, SIR, SPR, SGND, SYGE, SKFE, SQAE, GCTFSS, GTCKGH, GGRDGN, GAHNTS, GLAPSV, GAVDGI, GSVSSV, LVTAAGDQTAKFWDV and VSAGAVDGIIKVWDL of SEQ ID NO: 2 as noted above. Alternatively polypeptides having other combinations of the biological motifs disclosed herein are also contemplated such as a polypeptide having NMTG and NSTF, or a polypeptide having SIK and SPK etc of SEQ ID NO: 2 as noted above.

Polypeptides consisting of one or more of the 83P5G4 motifs discussed above are useful in elucidating the specific characteristics of a malignant phenotype in view of the observation that the 83P5G4 motifs discussed above are associated with growth disregulation and because 83P5G4 is highly expressed in multiple cancers (FIGS. 4-10). Casein kinase II, cAMP and cCMP-dependent protein kinase and Protein Kinase C for example are enzymes known to be associated with the development of the malignant phenotype (see e.g. Chen et al., Lab Invest., 78(2):165-174 (1998); Gaiddon et al., Endocrinology 136(10):4331-4338 (1995); Hall et al., Nucleic Acids Research 24(6):1119-1126 (1996); Peterziel et al., Oncogene 18(46):6322-6329 (1999) and O'Brian, Oncol. Rep. 5(2):305-309 (1998)). Moreover, both glycosylation and myristylation are protein modifications also associated with cancer and cancer progression (see e.g. Dennis et al., Biochem. Biophys. Acta 1473(1):21-34 (1999); Raju et al., Exp. Cell Res. 235(1):145-154 (1997)). Amidation is another protein modification also associated with cancer and cancer progression (see e.g. Treston et al., J. Natl. Cancer Inst. Monogr. (13):169-175 (1992)). In addition, nuclear localization sequences are also believed to influence the malignant potential of a cell (see e.g. Mirski et al., Cancer Res. 55(10):2129-2134 (1995)).

In another embodiment, proteins of the invention comprise one or more of the immunoreactive epitopes identified by a process described herein such as such as those shown in Tables IV-XVII. Processes for identifying peptides and analogs having affinities for HLA molecules and which are correlated as immunogenic epitopes, are well-known in the art. Also disclosed are principles for creating analogs of such epitopes in order to modulate inmmunogenicity. A variety of references are useful in the identification of such molecules. See, for example, WO 9733602 to Chesnut et al.; Sette, Immunogenetics 1999 50(3-4): 201-212; Sette et al., J. Immunol. 2001 166(2):1389-1397; Alexander et al., Immunol. Res. 18(2):79-92; Sidney et al., Hum. Immunol. 1997 58(1):12-20; Kondo et al., Immunogenetics 1997 45(4):249-258; Sidney et al., J. Immunol. 1996 157(8): 3480-90; and Falk et al., Nature 351:290-6 (1991); Hunt et al., Science 255:1261-3 (1992); Parker et al., J. Immunol. 149:3580-7 (1992); Parker et al., J. Immunol. 152:163-75 (1994)); Kast et al., 1994 152(8):3904-12; Borras-Cuesta et al., Hum. Immunol. 2000 61(3):266-278; Alexander et al., J. Immunol. 2000 164(3); 164(3): 1625-1633; Alexander et al., PMID:7895164, UI: 95202582; O'Sullivan et al., J. Immunol. 1991 147(8):2663-2669; Alexander et al., Immunity 1994 1(9):751-761 and Alexander et al., Immunol. Res. 1998 18(2): 79-92.

Related embodiments of the invention comprise polypeptides containing combinations of the different motifs discussed herein, where certain embodiments contain no insertions, deletions or substitutions either within the motifs or the intervening sequences of these polypeptides. In addition, embodiments which include a number of either N-terminal and/or C-terminal amino acid residues on either side of these motifs may be desirable (to, for example, include a greater portion of the polypeptide architecture in which the motif is located). Typically the number of N-terminal and/or C-terminal amino acid residues on either side of a motif is between about 1 to about 100 amino acid residues, preferably 5 to about 50 amino acid residues.

The proteins of the invention have a number of different specific uses. As 83P5G4 is shown to be highly expressed in prostate and other cancers (FIGS. 4-9), these peptides/proteins are used in methods that assess the status of 83P5G4 gene products in normal versus cancerous tissues and elucidating the malignant phenotype. Typically, polypeptides encoding specific regions of the 83P5G4 protein are used to assess the presence of perturbations (such as deletions, insertions, point mutations etc.) in specific regions (such as regions containing a nuclear localization signal) of the 83P5G4 gene products. Exemplary assays utilize antibodies or T cells targeting 83P5G4-related proteins comprising the amino acid residues of one or more of the biological motifs contained within the 83P5G4 polypeptide sequence in order to evaluate the characteristics of this region in normal versus cancerous tissues or to elicit an immune response to the epitope. Alternatively, 83P5G4 polypeptides containing the amino acid residues of one or more of the biological motifs contained within the 83P5G4 proteins are used to screen for factors that interact with that region of 83P5G4.

As discussed herein, redundancy in the genetic code permits variation in 83P5G4 gene sequences. In particular, it is known in the art that specific host species often have specific codon preferences, and thus one can adapt the disclosed sequence as preferred for a desired host. For example, preferred analog codon sequences typically have rare codons (i.e., codons having a usage frequency of less than about 20% in known sequences of the desired host) replaced with higher frequency codons. Codon preferences for a specific species are calculated, for example, by utilizing codon usage tables available on the INTERNET such as: http://www.dna.affrc.go.jp/˜nakamura/codon.html.

Additional sequence modifications are known to enhance protein expression in a cellular host. These include elimination of sequences encoding spurious polyadenylation signals, exon/intron splice site signals, transposon-like repeats, and/or other such well-characterized sequences that are deleterious to gene expression. The GC content of the sequence is adjusted to levels average for a given cellular host, as calculated by reference to known genes expressed in the host cell. Where possible, the sequence is modified to avoid predicted hairpin secondary mRNA structures. Other useful modifications include the addition of a translational initiation consensus sequence at the start of the open reading frame, as described in Kozak, Mol. Cell Biol., 9:5073-5080 (1989). Skilled artisans understand that the general rule that eukaryotic ribosomes initiate translation exclusively at the 5′ proximal AUG codon is abrogated only under rare conditions (see, e.g., Kozak PNAS 92(7):2662-2666, (1995) and Kozak NAR 15(20):8125-8148 (1987)).

83P5G4 proteins are embodied in many forms, preferably in isolated form. A purified 83P5G4 protein molecule will be substantially free of other proteins or molecules that impair the binding of 83P5G4 to antibody, T cell or other ligand. The nature and degree of isolation and purification will depend on the intended use. Embodiments of a 83P5G4 protein include a purified 83P5G4 protein and a functional, soluble 83P5G4 protein. In one embodiment, a functional, soluble 83P5G4 protein or fragment thereof retains the ability to be bound by antibody, T cell or other ligand.

The invention also provides 83P5G4 proteins comprising biologically active fragments of the 83P5G4 amino acid sequence corresponding to part of the 83P5G4 amino acid sequence shown in FIG. 2. Such proteins of the invention exhibit properties of the 83P5G4 protein, such as the ability to elicit the generation of antibodies that specifically bind an epitope associated with the 83P5G4 protein; to be bound by such antibodies; to elicit the activation of HTL or CTL; and/or, to be recognized by HTL or CTL.

83P5G4-related proteins are generated using standard peptide synthesis technology or using chemical cleavage methods well-known in the art. Alternatively, recombinant methods can be used to generate nucleic acid molecules that encode a 83P5G4-related protein. In one embodiment, the 83P5G4-encoding nucleic acid molecules provide means to generate defined fragments of 83P5G4 proteins. 83P5G4 protein fragments/subsequences are particularly useful in generating and characterizing domain-specific antibodies (e.g., antibodies recognizing an extracellular or intracellular epitope of a 83P5G4 protein), in identifying agents or cellular factors that bind to 83P5G4 or a particular structural domain thereof, and in various therapeutic contexts, including but not limited to cancer vaccines or methods of preparing such vaccines.

83P5G4 polypeptides containing particularly interesting structures can be predicted and/or identified using various analytical techniques well-known in the art, including, for example, the methods of Chou-Fasman, Garnier-Robson, Kyte-Doolittle, Eisenberg, Karplus-Schultz or Jameson-Wolf analysis, or on the basis of inmmunogenicity. Fragments containing such structures are particularly useful in generating subunit-specific anti-83P5G4 antibodies, or T cells or in identifying cellular factors that bind to 83P5G4.

Illustrating this, the binding of peptides from 83P5G4 proteins to the human MHC class I molecule HLA-A1, A2, A3, A11, A24, B7 and B35 were predicted. Specifically, the complete amino acid sequence of the 83P5G4 protein was entered into the HLA Peptide Motif Search algorithm found in the Bioinformatics and Molecular Analysis Section (BIMAS) Web site (http://bimas.dcrt.nih.gov/). The HLA Peptide Motif Search algorithm was developed by Dr. Ken Parker based on binding of specific peptide sequences in the groove of HLA Class I molecules and specifically HLA-A2 (see, e.g., Falk et al., Nature 351: 290-6 (1991); Hunt et al., Science 255:1261-3 (1992); Parker et al., J. Immunol. 149:3580-7 (1992); Parker et al., J. Immunol. 152:163-75 (1994)). This algorithm allows location and ranking of 8-mer, 9-mer, and 10-mer peptides from a complete protein sequence for predicted binding to HLA-A2 as well as numerous other HLA Class I molecules. Many HLA class I binding peptides are 8-, 9-, 10 or 11-mers. For example, for class I HLA-A2, the epitopes preferably contain a leucine (L) or methionine (M) at position 2 and a valine (V) or leucine (L) at the C-terminus (see, e.g., Parker et al., J. Immunol. 149:3580-7 (1992)).

Selected results of 83P5G4 predicted binding peptides are shown in Tables IV-XVII herein. It is to be appreciated that every epitope predicted by the BIMAS site, or specified by the HLA class I or class I motifs available in the art or which become part of the art are to be applied (e.g., visually or by computer-based methods, as appreciated by those of skill in the relevant art) are within the scope of the invention. In Tables IV-XVII, the top 50 ranking candidates, 9-mers and 10-mers, for each family member are shown along with their location, the amino acid sequence of each specific peptide, and an estimated binding score. The binding score corresponds to the estimated half-time of dissociation of complexes containing the peptide at 37° C. at pH 6.5. Peptides with the highest binding score are predicted to be the most tightly bound to HLA Class I on the cell surface for the greatest period of time and thus represent the best immunogenic targets for T-cell recognition. Actual binding of peptides to an HLA allele can be evaluated by stabilization of HLA expression on the antigen-processing defective cell line T2 (see, e.g., Xue et al., Prostate 30:73-8 (1997) and Peshwa et al., Prostate 36:129-38 (1998)). Immunogenicity of specific peptides can be evaluated in vitro by stimulation of CD8+cytotoxic T lymphocytes (CTL) in the presence of antigen presenting cells such as dendritic cells.

In an embodiment described in the examples that follow, 83P5G4 can be conveniently expressed in cells (such as 293T cells) transfected with a commercially available expression vector such as a CMV-driven expression vector encoding 83P5G4 with a C-terminal 6XHis and MYC tag (pcDNA3.1/mycHIS, Invitrogen or Tag5, GenHunter Corporation, Nashville Tenn.). The Tag5 vector provides an IgGK secretion signal that can be used to facilitate the production of a secreted 83P5G4 protein in transfected cells. The secreted HIS-tagged 83P5G4 in the culture media can be purified, e.g., using a nickel column using standard techniques.

Modifications of 83P5G4-related proteins such as covalent modifications are included within the scope of this invention. One type of covalent modification includes reacting targeted amino acid residues of an 83P5G4 polypeptide with an organic derivatizing agent that is capable of reacting with selected side chains or the N- or C- terminal residues of the 83P5G4. Another type of covalent modification of the 83P5G4 polypeptide included within the scope of this invention comprises altering the native glycosylation pattern of a protein of the invention. Another type of covalent modification of 83P5G4 comprises linking the 83P5G4 polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The 83P5G4-related proteins of the present invention can also be modified to form a chimeric molecule comprising 83P5G4 fused to another, heterologous polypeptide or amino acid sequence. Such a chimeric molecule can be synthesized chemically or recombinantly. A chimeric molecule can have a protein of the invention fused to another tumor-associated antigen or fragment thereof, or can comprise fusion of fragments of the 83P5G4 sequence (amino or nucleic acid) such that a molecule is created that is not, through its length, directly homologous to the amino or nucleic acid sequences respectively of FIG. 2 (SEQ ID NO: 2). Such a chimeric molecule can comprise multiples of the same subsequence of 83P5G4. A chimeric molecule can comprise a fusion of a 83P5G4-related protein with a polyhistidine epitope tag, which provides an epitope to which immobilized nickel can selectively bind. The epitope tag is generally placed at the amino- or carboxyl- terminus of the 83P5G4. In an alternative embodiment, the chimeric molecule can comprise a fusion of a 83P5G4-related protein with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of an 83P5G4 polypeptide in place of at least one variable region within an Ig molecule. In a particularly preferred embodiment, the immunoglobulin fusion includes the hinge, CH2 and CH3, or the hinge, CHI, CH2 and CH3 regions of an IgGI molecule. For the production of immunoglobulin fusions see also U.S. Pat. No. 5,428,130 issued Jun. 27, 1995.

### 83P5G4 ANTIBODIES

Another aspect of the invention provides antibodies that bind to 83P5G4-related proteins and polypeptides. Preferred antibodies specifically bind to an 83P5G4-related protein and do not bind (or bind weakly) to non-83P5G4 proteins. For example, antibodies bind 83P5G4-related proteins as well as the homologs or analogs thereof.

83P5G4 antibodies of the invention are particularly useful in prostate cancer diagnostic and prognostic assays, and imaging methodologies. Similarly, such antibodies are useful in the treatment, diagnosis, and/or prognosis of other cancers, to the extent 83P5G4 is also expressed or overexpressed in these other cancers. Moreover, intracellularly expressed antibodies (e.g., single chain antibodies) are therapeutically useful in treating cancers in which the expression of 83P5G4 is involved, such as for example advanced and metastatic prostate cancers.

The invention also provides various immunological assays useful for the detection and quantification of 83P5G4 and mutant 83P5G4-related proteins. Such assays can comprise one or more 83P5G4 antibodies capable of recognizing and binding an 83P5G4 or mutant 83P5G4 protein, as appropriate. These assays are performed within various immunological assay formats well-known in the art, including but not limited to various types of radioimmunoassays, enzyme-linked immunosorbent assays (ELISA), enzyme-linked immunofluorescent assays (ELIFA), and the like.

Immunological non-antibody assays of the invention also comprise T cell immunogenicity assays (inhibitory or stimulatory) as well as major bistocompatibility complex (MHC) binding assays. In addition, immunological imaging methods capable of detecting prostate cancer and other cancers expressing 83P5G4 are also provided by the invention, including but not limited to radioscintigraphic imaging methods using labeled 83P5G4 antibodies. Such assays are clinically useful in the detection, monitoring, and prognosis of 83P5G4-expressing cancers such as prostate cancer.

83P5G4 antibodies are also used in methods for purifying 83P5G4 and mutant 83P5G4 proteins and polypeptides and for isolating 83P5G4 homologues and related molecules. For example, a method of purifying a 83P5G4 protein comprises incubating an 83P5G4 antibody, which has been coupled to a solid matrix, with a lysate or other solution containing 83P5G4 under conditions that permit the 83P5G4 antibody to bind to 83P5G4; washing the solid matrix to eliminate impurities; and eluting the 83P5G4 from the coupled antibody. Other uses of the 83P5G4 antibodies of the invention include generating anti-idiotypic antibodies that mimic the 83P5G4 protein.

Various methods for the preparation of antibodies are well-known in the art. For example, antibodies can be prepared by immunizing a suitable mammalian host using an 83P5G4-related protein, peptide, or fragment, in isolated or immunoconjugated form (Antibodies: A Laboratory Manual, CSH Press, Eds., Harlow, and Lane (1988); Harlow, Antibodies, Cold Spring Harbor Press, NY (1989)). In addition, fusion proteins of 83P5G4 can also be used, such as an 83P5G4 GST-fusion protein. In a particular embodiment, a GST fusion protein comprising all or most of the open reading frame amino acid sequence of FIG. 2 is produced, then used as an immunogen to generate appropriate antibodies. In another embodiment, an 83P5G4 peptide is synthesized and used as an immunogen.

In addition, naked DNA immunization techniques known in the art are used (with or without purified 83P5G4 protein or 83P5G4-expressing cells) to generate an immune response to the encoded immunogen (for review, see Donnelly et al., 1997, Ann. Rev. Immunol. 15: 617-648).

The amino acid sequence of 83P5G4 as shown in FIG. 2 can be analyzed to select specific regions of the 83P5G4 protein for generating antibodies. For example, hydrophobicity and hydrophilicity analyses of the 83P5G4 amino acid sequence are used to identify hydrophilic regions in the 83P5G4 structure. Regions of the 83P5G4 protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Thus, each region identified by any of these programs/methods is within the scope of the present invention. Methods for the generation of 83P5G4 antibodies are further illustrated by way of the examples provided herein.

Methods for preparing a protein or polypeptide for use as an immunogen and for preparing immunogenic conjugates of a protein with a carrier such as BSA, KLH, or other carrier proteins are well-known in the art. In some circumstances, direct conjugation using, for example, carbodiimide reagents are used; in other instances linking reagents such as those supplied by Pierce Chemical Co., Rockford, Ill., are effective. Administration of an 83P5G4 immunogen is conducted generally by injection over a suitable time period and with use of a suitable adjuvant, as is generally understood in the art. During the immunization schedule, titers of antibodies can be taken to determine adequacy of antibody formation.

83P5G4 monoclonal antibodies can be produced by various means well-known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay in which the antigen is a 83P5G4-related protein. When the appropriate immortalized cell culture is identified, the cells can be expanded and antibodies produced either from in vitro cultures or from ascites fluid.

The antibodies or fragments can also be produced, using current technology, by recombinant means. Regions that bind specifically to the desired regions of the 83P5G4 protein can also be produced in the context of chimeric or complementarity determining region (CDR) grafted antibodies of multiple species origin. Humanized or human 83P5G4 antibodies can also be produced and are preferred for use in therapeutic contexts. Methods for humanizing murine and other non-human antibodies, by substituting one or more of the non-human antibody CDRs for corresponding human antibody sequences, are well-known (see for example, Jones et al., 1986, Nature 321:522-525; Riechmnan et al., 1988, Nature 332:323-327; Verhoeyen et al., 1988, Science 239:1534-1536). See also, Carter et al., 1993, Proc. Natl. Acad. Sci. USA 89:4285 and Sims et al., 1993, J. Immunol. 151:2296.

Methods for producing fully human monoclonal antibodies include phage display and transgenic methods (for review, see Vaughan et al., 1998, Nature Biotechnology 16:535-539). Fully human 83P5G4 monoclonal antibodies can be generated using cloning technologies employing large human Ig gene combinatorial libraries (i.e., phage display) (Griffiths and Hoogenboom, Building an in vitro immune system: human antibodies from phage display libraries. In: Protein Engineering of Antibody Molecules for Prophylactic and Therapeutic Applications in Man. Clark, M. (Ed.), Nottingham Academic, pp 45-64 (1993); Burton and Barbas, Human Antibodies from combinatorial libraries. Id., pp 65-82). Fully human 83P5G4 monoclonal antibodies can also be produced using transgenic mice engineered to contain human immunoglobulin gene loci as described in PCT Patent Application WO98/24893, Kucherlapati and Jakobovits et al., published Dec. 3, 1997 (see also, Jakobovits, 1998, Exp. Opin. Invest. Drugs 7(4):607-614; U.S. Pat. Nos. 6,162,963 issued Dec. 19, 2000; 6,150,584 issued Nov. 12, 2000; and, 6,114,598 issued Sep. 5, 2000). This method avoids the in vitro manipulation required with phage display technology and efficiently produces high affinity authentic human antibodies.

Reactivity of 83P5G4 antibodies with a 83P5G4-related protein can be established by a number of well-known means, including Western blot, immunoprecipitation, ELISA, and FACS analyses using, as appropriate, 83P5G4-related proteins, 83P5G4-expressing cells or extracts thereof.

An 83P5G4 antibody or fragment thereof is labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more 83P5G4 epitopes are generated using methods generally known in the art. Homodimeric antibodies can also be generated by cross-linking techniques known in the art (e.g., Wolff et al., Cancer Res. 53:2560-2565).

### 83P5G4 TRANSGENIC ANIMALS

Nucleic acids that encode 83P5G4 or its modified forms can also be used to generate either transgenic animals or “knock out” animals which, in turn, are useful in the development and screening of therapeutically useful reagents. In accordance with established techniques, cDNA encoding 83P5G4 can be used to clone genomic DNA that encodes 83P5G4. The cloned genomic sequences can then be used to generate transgenic animals that contain cells that express DNA encoding 83P5G4. Methods for generating transgenic animals, particularly animals such as mice or rats, have become conventional in the art and are described, for example, in U.S. Pat. Nos. 4,736,866 issued Apr. 12, 1988, and 4,870,009 issued Sep. 26, 1989. Typically, particular cells would be targeted for 83P5G4 transgene incorporation with tissue-specific enhancers.

Transgenic animals that include a copy of a transgene encoding 83P5G4 can be used to examine the effect of increased expression of DNA that encodes 83P5G4. Such animals can be used as tester animals for reagents thought to confer protection from, for example, pathological conditions associated with its overexpression. In accordance with this facet of the invention, an animal is treated with a reagent and a reduced incidence of the pathological condition, compared to untreated animals that bear the transgene, would indicate a potential therapeutic intervention for the pathological condition.

Alternatively, non-human homologues of 83P5G4 can be used to construct an 83P5G4 “knock out” animal that has a defective or altered gene encoding 83P5G4 as a result of homologous recombination between the endogenous gene encoding 83P5G4 and altered genomic DNA encoding 83P5G4 introduced into an embryonic cell of the animal. For example, cDNA that encodes 83P5G4 can be used to clone genomic DNA encoding 83P5G4 in accordance with established techniques. A portion of the genomic DNA encoding 83P5G4 can be deleted or replaced with another gene, such as a gene encoding a selectable marker that can be used to monitor integration. Typically, several kilobases of unaltered flanking DNA (both at the 5′ and 3′ ends) are included in the vector [see, e.g.,, Thomas and Capecchi, Cell 51:503 (1987) for a description of homologous recombination vectors]. The vector is introduced into an embryonic stem cell line (e.g., by electroporation) and cells in which the introduced DNA has homologously recombined with the endogenous DNA are selected [see, e.g.,, Li et al., Cell, 69:915 (1992)]. The selected cells are then injected into a blastocyst of an animal (e.g., a mouse or rat) to form aggregation chimeras [see, e.g.,, Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knock out animals can be characterized for instance, for their ability to defend against certain pathological conditions or for their development of pathological conditions due to absence of the 83P5G4 polypeptide.

### METHODS FOR THE DETECTION OF 83P5G4

Another aspect of the present invention relates to methods for detecting 83P5G4 polynucleotides and 83P5G4-related proteins and variants thereof, as well as methods for identifying a cell that expresses 83P5G4. 83P5G4 appears to be expressed in the LAPC xenografts that are derived from lymph node and bone metastasis of prostate cancer. The expression profile of 83P5G4 makes it a diagnostic marker for metastasized disease. Accordingly, the status of 83P5G4 gene products provides information useful for predicting a variety of factors including susceptibility to advanced stage disease, rate of progression, and/or tumor aggressiveness. As discussed in detail herein, the status of 83P5G4 gene products in patient samples can be analyzed by a variety protocols that are well-known in the art including immunohistochemical analysis, the variety of Northern blotting techniques including in situ hybridization, RT-PCR analysis (for example on laser capture micro-dissected samples), Western blot analysis and tissue array analysis.

More particularly, the invention provides assays for the detection of 83P5G4 polynucleotides in a biological sample, such as serum, bone, prostate, and other tissues, urine, semen, cell preparations, and the like. Detectable 83P5G4 polynucleotides include, for example, an 83P5G4 gene or fragment thereof, 83P5G4 mRNA, alternative splice variant 83P5G4 mRNAs, and recombinant DNA or RNA molecules containing a 83P5G4 polynucleotide. A number of methods for amplifying and/or detecting the presence of 83P5G4 polynucleotides are well-known in the art and can be employed in the practice of this aspect of the invention.

In one embodiment, a method for detecting an 83P5G4 mRNA in a biological sample comprises producing cDNA from the sample by reverse transcription using at least one primer; amplifying the cDNA so produced using a 83P5G4 polynucleotides as sense and antisense primers to amplify 83P5G4 cDNAs therein; and detecting the presence of the amplified 83P5G4 cDNA. Optionally, the sequence of the amplified 83P5G4 cDNA can be determined.

In another embodiment, a method of detecting an 83P5G4 gene in a biological sample comprises first isolating genomic DNA from the sample; amplifying the isolated genomic DNA using 83P5G4 polynucleotides as sense and antisense primers; and detecting the presence of the amplified 83P5G4 gene. Any number of appropriate sense and antisense probe combinations can be designed from the nucleotide sequences provided for the 83P5G4 (FIG. 2) and used for this purpose.

The invention also provides assays for detecting the presence of a 83P5G4 protein in a tissue of other biological sample such as serum, bone, prostate, and other tissues, urine, cell preparations, and the like. Methods for detecting a 83P5G4 protein are also well-known and include, for example, immunoprecipitation, immunohistochemical analysis, Western Blot analysis, molecular binding assays, ELISA, ELIFA and the like. For example, in one embodiment, a method of detecting the presence of a 83P5G4 protein in a biological sample comprises first contacting the sample with an 83P5G4 antibody, an 83P5G4-reactive fragment thereof, or a recombinant protein containing an antigen-binding region of an 83P5G4 antibody; and then detecting the binding of 83P5G4 protein in the sample thereto.

Methods for identifying a cell that expresses 83P5G4 are also provided. In one embodiment, an assay for identifying a cell that expresses an 83P5G4 gene comprises detecting the presence of 83P5G4 mRNA in the cell. Methods for the detection of particular mRNAs in cells are well-known and include, for example, hybridization assays using complementary DNA probes (such as in situ hybridization using labeled 83P5G4 riboprobes, Northern blot and related techniques) and various nucleic acid amplification assays (such as RT-PCR using complementary primers specific for 83P5G4, and other amplification type detection methods, such as, for example, branched DNA, SISBA, TMA and the like). Alternatively, an assay for identifying a cell that expresses an 83P5G4 gene comprises detecting the presence of 83P5G4 protein in the cell or secreted by the cell. Various methods for the detection of proteins are well-known in the art and are employed for the detection of 83P5G4 proteins and 83P5G4-expressing cells. 83P5G4 expression analysis is also useful as a tool for identifying and evaluating agents that modulate 83P5G4 gene expression. For example, 83P5G4 expression is significantly upregulated in prostate cancer, and is expressed in cancers of the tissues listed in Table 1. Identification of a molecule or biological agent that inhibits 83P5G4 expression or over-expression in cancer cells is of therapeutic value. For example, such an agent can be identified by using a screen that quantifies 83P5G4 expression by RT-PCR, nucleic acid hybridization or antibody binding.

### MONITORING THE STATUS OF 83P5G4 AND ITS PRODUCTS

Assays that evaluate the status of the 83P5G4 gene and 83P5G4 gene products in an individual provide information on the growth or oncogenic potential of a biological sample from this individual. For example, because 83P5G4 mRNA is so highly expressed in prostate cancers (as well as the other cancer tissues shown for example in FIGS. 4-9 and Table I) as compared to normal prostate tissue, assays that evaluate the relative levels of 83P5G4 mRNA transcripts or proteins in a biological sample can be used to diagnose a disease associated with 83P5G4 disregulation such as cancer and can provide prognostic information useful in defining appropriate therapeutic options.

Because 83P5G4 is expressed, for example, in various prostate cancer tissues, xenografts and cancer cell lines, and cancer patient samples, the expression status of 83P5G4 provides information including the presence, stage and location of dysplastic, precancerous and cancerous cells, predicting susceptibility to various stages of disease, and/or for gauging tumor aggressiveness. Moreover, the expression profile makes it useful as an imaging reagent for metastasized disease. Consequently, an important aspect of the invention is directed to the various molecular prognostic and diagnostic methods for examining the status of 83P5G4 in biological samples such as those from individuals suffering from, or suspected of suffering from a pathology characterized by disregulated cellular growth such as cancer.

Oncogenesis is known to be a multistep process where cellular growth becomes progressively disregulated and cells progress from a normal physiological state to precancerous and then cancerous states (see, e.g., Alers et al., Lab Invest. 77(5):437-438 (1997) and Isaacs et al., Cancer Surv. 23:19-32 (1995)). In this context, examining a biological sample for evidence of disregulated cell growth (such as aberrant 83P5G4 expression in prostate cancers) allows for early detection of such aberrant cellular physiology, before a pathology such as cancer has progressed to a stage at which therapeutic options are more limited. In such examinations, the status of 83P5G4 in a biological sample of interest can be compared, for example, to the status of 83P5G4 in a corresponding normal sample (e.g. a sample from that individual or alternatively another individual that is not effected by a pathology). Alterations in the status of 83P5G4 in the biological sample of interest (as compared to the normal sample) provides evidence of disregulated cellular growth. In addition to using a biological sample that is not effected by a pathology as a normal sample, one can also use a predetermined normative value such as a predetermined normal level of mRNA expression (see, e.g., Grever et al., J. Comp. Neurol. Dec. 9, 1996;376(2):306-14 and U.S. Pat. No. 5,837,501) to compare 83P5G4 in normal versus suspect samples.

The term “status” in this context is used according to its art accepted meaning and refers to the condition or state of a gene and its products. Typically, skilled artisans use a number of parameters to evaluate the condition or state of a gene and its products. These include, but are not limited to the location of expressed gene products (including the location of 83P5G4-expressing cells) as well as the, level, and biological activity of expressed gene products (such as 83P5G4 mRNA polynucleotides and polypeptides). Typically, an alteration in the status of 83P5G4 comprises a change in the location of 83P5G4 and/or 83P5G4-expressing cells and/or an increase in 83P5G4 mRNA and/or protein expression.

Moreover, in order to identify a condition or phenomenon associated with disregulated cell growth, the status of 83P5G4 in a biological sample is evaluated by various methods utilized by skilled artisans including, but not limited to genomic Southern analysis (to examine, for example perturbations in the 83P5G4 gene), Northern analysis and/or PCR analysis of 83P5G4 mRNA (to examine, for example alterations in the polynucleotide sequences or expression levels of 83P5G4 mRNAs), and, Western and/or immunohistochemical analysis (to examine, for example alterations in polypeptide sequences, alterations in polypeptide localization within a sample, alterations in expression levels of 83P5G4 proteins and/or associations of 83P5G4 proteins with polypeptide binding partners). Detectable 83P5G4 polynucleotides include, for example, an 83P5G4 gene or fragment thereof, 83P5G4 mRNA, alternative splice variants 83P5G4 mRNAs, and recombinant DNA or RNA molecules containing a 83P5G4 polynucleotide.

The expression profile of 83P5G4 makes it a diagnostic marker for local and/or metastasized disease. In particular, the status of 83P5G4 provides information useful for predicting susceptibility to particular disease stages, progression, and/or tumor aggressiveness. The invention provides methods and assays for determining 83P5G4 status and diagnosing cancers that express 83P5G4, such as cancers of the tissues listed in Table I. 83P5G4 status in patient samples can be analyzed by a number of means well-known in the art, including without limitation, immunohistochemical analysis, in situ hybridization, RT-PCR analysis on laser capture micro-dissected samples, Western blot analysis of clinical samples and cell lines, and tissue array analysis. Typical protocols for evaluating the status of the 83P5G4 gene and gene products are found, for example in Ausubul et al. eds., 1995, Current Protocols In Molecular Biology, Units 2 [Northern Blotting], 4 [Southern Blotting], 15 [Immunoblotting] and 18 [PCR Analysis].

As described above, the status of 83P5G4 in a biological sample can be examined by a number of well-known procedures in the art. For example, the status of 83P5G4 in a biological sample taken from a specific location in the body can be examined by evaluating the sample for the presence or absence of 83P5G4-expressing cells (e.g. those that express 83P5G4 mRNAs or proteins). This examination can provide evidence of disregulated cellular growth, for example, when 83P5G4-expressing cells are found in a biological sample that does not normally contain 83P5G4-expressing cells (or contains cells that express specific isoforms of 83P5G4 mRNAs) is found to contain 83P5G4-expressing cells (or cells that express different isoforms of 83P5G4 mRNAs) (such as a lymph node). Such alterations in the status of 83P5G4 in a biological sample are often associated with disregulated cellular growth. Specifically, one indicator of disregulated cellular growth is the metastases of cancer cells from an organ of origin (such as the bladder or prostate gland) to a different area of the body (such as a lymph node). In this context, evidence of disregulated cellular growth is important for example because occult lymph node metastases can be detected in a substantial proportion of patients with prostate cancer, and such metastases are associated with known predictors of disease progression (see, e.g., Murphy et al., Prostate 42(4):315-317 (2000);Su et al., Semin. Surg. Oncol. 18(1):17-28 (2000) and Freeman et al., J Urol 1995 Aug;154(2 Pt 1):474-8).

In one aspect, the invention provides methods for monitoring 83P5G4 gene products by determining the status of 83P5G4 gene products expressed by cells in from an individual suspected of having a disease associated with disregulated cell growth (such as hyperplasia or cancer) and then comparing the status so determined to the status of 83P5G4 gene products in a corresponding normal sample. The presence of aberrant 83P5G4 gene products in the test sample relative to the normal sample provides an indication of the presence of disregulated cell growth within the cells of the individual.

In a specific embodiment of the invention, one can monitor different 83P5G4 mRNAs, such as the 1.8, 2.5 and 4.5 KB transcripts that are expressed in different cancers as shown for example in FIGS. 4-10. The monitoring of alternative splice variants of 83P5G4 is useful because changes in the alternative splicing of mRNAs is suggested as one of the steps in a series of events that lead to the progression of cancers (see e.g. Carstens et al., Oncogene 15(25):3059-3065 (1997)). Consequently, monitoring of alternative splice variants of 83P5G4 provides an additional means to evaluate syndromes associated with perturbations in 83P5G4 gene products such as cancers.

In other related embodiments, one can evaluate the status 83P5G4 nucleotide and amino acid sequences in a biological sample in order to identify perturbations in the structure of these molecules such as insertions, deletions, substitutions and the like. Such embodiments are useful because perturbations in the nucleotide and amino acid sequences are observed in a large number of proteins associated with a growth disregulated phenotype (see, e.g., Marrogi et al., 1999, J. Cutan. Pathol. 26(8):369-378). For example, a mutation in the sequence of 83P5G4 may be indicative of the presence or promotion of a tumor. Such assays therefore have diagnostic and predictive value where a mutation in 83P5G4 indicates a potential loss of function or increase in tumor growth.

A wide variety of assays for observing perturbations in nucleotide and amino acid sequences are well-known in the art. For example, the size and structure of nucleic acid or amino acid sequences of 83P5G4 gene products are observed by the Northern, Southern, Western, PCR and DNA sequencing protocols discussed herein. In addition, other methods for observing perturbations in nucleotide and amino acid sequences such as single strand conformation polymorphism analysis are well-known in the art (see, e.g., U.S. Pat. Nos. 5,382,510 issued Sep. 7,1999, and 5,952,170 issued Jan. 17, 1995).

In another embodiment, one can examine the methylation status of the 83P5G4 gene in a biological sample. Aberrant demethylation and/or hypermethylation of CpG islands in gene 5′ regulatory regions frequently occurs in immortalized and transformed cells and can result in altered expression of various genes. For example, promoter hypermethylation of the pi-class glutathione S-transferase (a protein expressed in normal prostate but not expressed in >90% of prostate carcinomas) appears to permanently silence transcription of this gene and is the most frequently detected genomic alteration in prostate carcinomas (De Marzo et al., Am. J. Pathol. 155(6):1985-1992 (1999)). In addition, this alteration is present in at least 70% of cases of high-grade prostatic intraepithelial neoplasia (PIN) (Brooks et al, Cancer Epidemiol. Biomarkers Prev., 1998, 7:531-536). In another example, expression of the LAGE-I tumor specific gene (which is not expressed in normal prostate but is expressed in 25-50% of prostate cancers) is induced by deoxy-azacytidine in lymphoblastoid cells, suggesting that tumoral expression is due to demethylation (Lethe et al., Int. J. Cancer 76(6):903-908 (1998)). A variety of assays for examining methylation status of a gene are well-known in the art. For example, one can utilize, in Southern hybridization approaches, methylation-sensitive restriction enzymes which cannot cleave sequences that contain methylated CpG sites, in order to assess the overall methylation status of CpG islands. In addition, MSP (methylation specific PCR) can rapidly profile the methylation status of all the CpG sites present in a CpG island of a given gene. This procedure involves initial modification of DNA by sodium bisulfite (which will convert all unmethylated cytosines to uracil) followed by amplification using primers specific for methylated versus unmethylated DNA. Protocols involving methylation interference can also be found for example in Current Protocols In Molecular Biology, Unit 12, Frederick M. Ausubul et al. eds., 1995.

Gene amplification provides an additional method of assessing the status of 83P5G4, a locus that maps to 1q31-1q32.1, a region shown to be perturbed in certain cancers. Gene amplification is measured in a sample directly, for example, by conventional Southern blotting or Northern blotting to quantitate the transcription of mRNA (Thomas, 1980, Proc. Natl. Acad. Sci. USA, 77:5201-5205), dot blotting (DNA analysis), or in situ hybridization, using an appropriately labeled probe, based on the sequences provided herein. Alternatively, antibodies are employed that recognize specific duplexes, including DNA duplexes, RNA duplexes, and DNA-RNA hybrid duplexes or DNA-protein duplexes. The antibodies in turn are labeled and the assay carried out where the duplex is bound to a surface, so that upon the formation of duplex on the surface, the presence of antibody bound to the duplex can be detected.

Biopsied tissue or peripheral blood can be conveniently assayed for the presence of cancer cells using for example, Northern, dot blot or RT-PCR analysis to detect 83P5G4 expression (see, e.g., FIGS. 4-9). The presence of RT-PCR amplifiable 83P5G4 mRNA provides an indication of the presence of cancer. RT-PCR assays are well-known in the art. RT-PCR detection assays for tumor cells in peripheral blood are currently being evaluated for use in the diagnosis and management of a number of human solid tumors. In the prostate cancer field, these include RT-PCR assays for the detection of cells expressing PSA and PSM (Verkaik et al., 1997, Urol. Res. 25:373-384; Ghossein et al., 1995, J. Clin. Oncol. 13:1195-2000; Heston et al., 1995, Clin. Chem. 41:1687-1688).

A related aspect of the invention is directed to predicting susceptibility of an individual for developing cancer. In one embodiment, a method for predicting susceptibility to cancer comprises detecting 83P5G4 mRNA or 83P5G4 protein in a tissue sample, its presence indicating susceptibility to cancer, wherein the degree of 83P5G4 mRNA expression correlates to the degree of susceptibility. In a specific embodiment, the presence of 83P5G4 in prostate or other tissue is examined, with the presence of 83P5G4 in the sample providing an indication of prostate cancer susceptibility (or the emergence or existence of a prostate tumor). In a closely related embodiment, one can evaluate the integrity 83P5G4 nucleotide and amino acid sequences in a biological sample in order to identify perturbations in the structure of these molecules such as insertions, deletions, substitutions and the like, with the presence of one or more perturbations in 83P5G4 gene products in the sample providing an indication of cancer susceptibility (or the emergence or existence of a tumor).

Another related aspect of the invention is directed to methods for gauging tumor aggressiveness. In one embodiment, a method for gauging aggressiveness of a tumor comprises determining the level of 83P5G4 mRNA or 83P5G4 protein expressed by tumor cells, comparing the level so determined to the level of 83P5G4 mRNA or 83P5G4 protein expressed in a corresponding normal tissue taken from the same individual or a normal tissue reference sample, wherein the degree of 83P5G4 mRNA or 83P5G4 protein expression in the tumor sample relative to the normal sample indicates the degree of aggressiveness. In a specific embodiment, aggressiveness of a tumor is evaluated by determining the extent to which 83P5G4 is expressed in the tumor cells, with higher expression levels indicating more aggressive tumors. In a closely related embodiment, one can evaluate the integrity of 83P5G4 nucleotide and amino acid sequences in a biological sample in order to identify perturbations in the structure of these molecules such as insertions, deletions, substitutions and the like, with the presence of one or more perturbations indicating more aggressive tumors.

Yet another related aspect of the invention is directed to methods for observing the progression of a malignancy in an individual over time. In one embodiment, methods for observing the progression of a malignancy in an individual over time comprise determining the level of 83P5G4 mRNA or 83P5G4 protein expressed by cells in a sample of the tumor, comparing the level so determined to the level of 83P5G4 mRNA or 83P5G4 protein expressed in an equivalent tissue sample taken from the same individual at a different time, wherein the degree of 83P5G4 mRNA or 83P5G4 protein expression in the tumor sample over time provides information on the progression of the cancer. In a specific embodiment, the progression of a cancer is evaluated by determining the extent to which 83P5G4 expression in the tumor cells alters over time, with higher expression levels indicating a progression of the cancer. Also, one can evaluate the integrity 83P5G4 nucleotide and amino acid sequences in a biological sample in order to identify perturbations in the structure of these molecules such as insertions, deletions, substitutions and the like, where the presence of one or more perturbations indicates a progression of the cancer.

The above diagnostic approaches can be combined with any one of a wide variety of prognostic and diagnostic protocols known in the art. For example, another embodiment of the invention is directed to methods for observing a coincidence between the expression of 83P5G4 gene and 83P5G4 gene products (or perturbations in 83P5G4 gene and 83P5G4 gene products) and a factor that is associated with malignancy, as a means for diagnosing and prognosticating the status of a tissue sample. A wide variety of factors associated with malignancy can be utilized, such as the expression of genes associated with malignancy (e.g. PSA, PSCA and PSM expression for prostate cancer etc.) as well as gross cytological observations (see, e.g., Bocking et al., 1984, Anal. Quant. Cytol. 6(2):74-88; Eptsein, 1995, Hum. Pathol. 26(2):223-9; Thorson et al., 1998, Mod. Pathol. 11(6):543-51; Baisden et al., 1999, Am. J. Surg. Pathol. 23(8):918-24). Methods for observing a coincidence between the expression of 83P5G4 gene and 83P5G4 gene products (or perturbations in 83P5G4 gene and 83P5G4 gene products) and another factor that is associated with malignancy are useful, for example, because the presence of a set of specific factors that coincide with disease provides information crucial for diagnosing and prognosticating the status of a tissue sample.

In a typical embodiment, methods for observing a coincidence between the expression of 83P5G4 gene and 83P5G4 gene products (or perturbations in 83P5G4 gene and 83P5G4 gene products) and another factor that is associated with malignancy entails detecting the overexpression of 83P5G4 mRNA or protein in a tissue sample, detecting the overexpression of PSA mRNA or protein in a tissue sample, and observing a coincidence of 83P5G4 mRNA or protein and PSA mRNA or protein overexpression. In a specific embodiment, the expression of 83P5G4 and PSA mRNA in prostate tissue is examined. In a preferred embodiment, the coincidence of 83P5G4 and PSA mRNA overexpression in the sample indicates the existence of prostate cancer, prostate cancer susceptibility or the emergence or status of a prostate tumor.

Methods for detecting and quantifying the expression of 83P5G4 mRNA or protein are described herein, and standard nucleic acid and protein detection and quantification technologies are well-known in the art. Standard methods for the detection and quantification of 83P5G4 mRNA include in situ hybridization using labeled 83P5G4 riboprobes, Northern blot and related techniques using 83P5G4 polynucleotide probes, RT-PCR analysis using primers specific for 83P5G4, and other amplification type detection methods, such as, for example, branched DNA, SISBA, TMA and the like. In a specific embodiment, semi-quantitative RT-PCR is used to detect and quantify 83P5G4 mRNA expression. Any number of primers capable of amplifying 83P5G4 can be used for this purpose, including but not limited to the various primer sets specifically described herein. Standard methods for the detection and quantification of protein are also used. In a specific embodiment, polyclonal or monoclonal antibodies specifically reactive with the wild-type 83P5G4 protein can be used in an imunohistochemical assay of biopsied tissue.

### IDENTIFYING MOLECULES THAT INTERACT WITH 83P5G4

The 83P5G4 protein sequences disclosed herein allow a skilled artisan to identify proteins, small molecules and other agents that interact with 83P5G4 and pathways activated by 83P5G4 via any one of a variety of art accepted protocols. For example, one can utilize one of the variety of so-called interaction trap systems (also referred to as the “two-hybrid assay”). In such systems, molecules that interact reconstitute a transcription factor, which directs expression of a reporter gene, whereupon the expression of the reporter gene is assayed. Typical systems identify protein-protein interactions in vivo through reconstitution of a eukaryotic transcriptional activator and are disclosed for example in U.S. Pat. Nos. 5,955,280 issued Sep. 21, 1999, 5,925,523 issued Jul. 20, 1999, 5,846,722 issued Dec. 8, 1998 and 6,004,746 issued Dec. 21, 1999.

Alternatively one can identify molecules that interact with 83P5G4 protein sequences by screening peptide libraries. In such methods, peptides that bind to selected receptor molecules such as 83P5G4 are identified by screening libraries that encode a random or controlled collection of amino acids. Peptides encoded by the libraries are expressed as fusion proteins of bacteriophage coat proteins; the bacteriophage particles are then screened against the receptors of interest.

Accordingly, peptides having a wide variety of uses, such as therapeutic, prognostic or diagnostic reagents, are thus identified without any prior information on the structure of the expected ligand or receptor molecule. Typical peptide libraries and screening methods that can be used to identify molecules that interact with 83P5G4 protein sequences are disclosed for example in U.S. Pat. Nos. 5,723,286 issued Mar. 3, 1998 and 5,733,731 issued Mar. 31, 1998.

Alternatively, cell lines that express 83P5G4 are used to identify protein-protein interactions mediated by 83P5G4. Such interactions can be examined using immunoprecipitation techniques as shown by others (Hamilton B J, et al. Biochem. Biophys. Res. Commun. 1999, 261:646-51). Typically 83P5G4 protein can be immunoprecipitated from 83P5G4-expressing prostate cancer cell lines using anti-83P5G4 antibodies. Alternatively, antibodies against His-tag can be used in a cell line engineered to express 83P5G4 (vectors mentioned above). The immunoprecipitated complex can be examined for protein association by procedures such as Western blotting, ³⁵S-methionine labeling of proteins, protein microsequencing, silver staining and two-dimensional gel electrophoresis.

Small molecules that interact with 83P5G4 can be identified through related embodiments of such screening assays. For example, small molecules can be identified that interfere with protein function, including molecules that interfere with 83P5G4's ability to mediate phosphorylation and de-phosphorylation, second messenger signaling and tumorigenesis. Typical methods are discussed for example in U.S. Pat. No. 5,928,868 issued Jul. 27, 1999, and include methods for forming hybrid ligands in which at least one ligand is a small molecule. In an illustrative embodiment, the hybrid ligand is introduced into cells that in turn contain a first and a second expression vector. Each expression vector includes DNA for expressing a hybrid protein that encodes a target protein linked to a coding sequence for a transcriptional module. The cells further contain a reporter gene, the expression of which is conditioned on the proximity of the first and second hybrid proteins to each other, an event that occurs only if the hybrid ligand binds to target sites on both hybrid proteins. Those cells that express the reporter gene are selected and the unknown small molecule or the unknown hybrid protein is identified.

An embodiment of this invention comprises a method of screening for a molecule that interacts with an 83P5G4 amino acid sequence shown in FIG. 2 (SEQ ID NO: 2), comprising the steps of contacting a population of molecules with the 83P5G4 amino acid sequence, allowing the population of molecules and the 83P5G4 amino acid sequence to interact under conditions that facilitate an interaction, determining the presence of a molecule that interacts with the 83P5G4 amino acid sequence and then separating molecules that do not interact with the 83P5G4 amino acid sequence from molecules that do interact with the 83P5G4 amino acid sequence. In a specific embodiment, the method further includes purifying a molecule that interacts with the 83P5G4 amino acid sequence. The identified molecule can be used to modulate a function performed by 83P5G4. In a preferred embodiment, the 83P5G4 amino acid sequence is contacted with a library of peptides.

### THERAPEUTIC METHODS AND COMPOSITIONS

The identification of 83P5G4 as a protein that is normally expressed in a restricted set of tissues and which is also expressed in prostate and other cancers, opens a number of therapeutic approaches to the treatment of such cancers. As discussed herein, it is possible that 83P5G4 functions as a transcription factor involved in activating tumor-promoting genes or repressing genes that block tumorigenesis.

Accordingly, therapeutic approaches that inhibit the activity of the 83P5G4 protein are useful for patients suffering from prostate cancer, testicular cancer, and other cancers expressing 83P5G4. These therapeutic approaches generally fall into two classes. One class comprises various methods for inhibiting the binding or association of the 83P5G4 protein with its binding partner or with others proteins. Another class comprises a variety of methods for inhibiting the transcription of the 83P5G4 gene or translation of 83P5G4 mRNA.

83P5G4 as a Target for Antibody-Based Therapy

83P5G4 is an attractive target for antibody-based therapeutic strategies. A number of antibody strategies are known in the art for targeting both extracellular and intracellular molecules (see, e.g., complement and ADCC mediated killing as well as the use of intrabodies discussed herein). Because 83P5G4 is expressed by cancer cells of various lineages and not by corresponding normal cells, systemic administration of 83P5G4-immunoreactive compositions are prepared that exhibit excellent sensitivity without toxic, non-specific and/or non-target effects caused by binding of the immunotherapeutic molecule to non-target organs and tissues. Antibodies specifically reactive with domains of 83P5G4 are useful to treat 83P5G4-expressing cancers systemically, either as conjugates with a toxin or therapeutic agent, or as naked antibodies capable of inhibiting cell proliferation or function.

83P5G4 antibodies can be introduced into a patient such that the antibody binds to 83P5G4 and modulates or perturbs a function, such as an interaction with a binding partner, and consequently mediates destruction of the tumor cells and/or inhibits the growth of the tumor cells. Mechanisms by which such antibodies exert a therapeutic effect can include complement-mediated cytolysis, antibody-dependent cellular cytotoxicity, modulating the physiological function of 83P5G4, inhibiting ligand binding or signal transduction pathways, modulating tumor cell differentiation, altering tumor angiogenesis factor profiles, and/or by inducing apoptosis.

Those skilled in the art understand that antibodies can be used to specifically target and bind immunogenic molecules such as an immunogenic region of the 83P5G4 sequence shown in FIG. 2. In addition, skilled artisans understand that it is routine to conjugate antibodies to cytotoxic agents. Skilled artisans understand that when cytotoxic and/or therapeutic agents are delivered directly to cells by conjugating them to antibodies specific for a molecule expressed by that cell (e.g. 83P5G4), it is reasonable to expect that the cytotoxic agent will exert its known biological effect (e.g. cytotoxicity) on those cells.

A wide variety of compositions and methods for using antibodies conjugated to cytotoxic agents to kill cells are known in the art. In the context of cancers, typical methods entail administering to an animal having a tumor a biologically effective amount of a conjugate comprising a selected cytotoxic and/or therapeutic agent linked to a targeting agent (e.g. an anti-83P5G4 antibody) that binds to a marker (e.g. 83P5G4) expressed, accessible to binding or localized on the cell surfaces. A typical embodiment consists of a method of delivering a cytotoxic and/or therapeutic agent to a cell expressing 83P5G4, comprising conjugating the cytotoxic agent to an antibody that immunospecifically binds to an 83P5G4 epitope, and, exposing the cell to the antibody-agent conjugate. Another specific illustrative embodiment consists of a method of treating an individual suspected of suffering from metastasized cancer, comprising a step of administering parenterally to said individual a pharmaceutical composition comprising a therapeutically effective amount of an antibody conjugated to a cytotoxic and/or therapeutic agent.

Cancer immunotherapy using anti-83P5G4 antibodies may follow the teachings generated from various approaches that have been successfully employed in the treatment of other types of cancer, including but not limited to colon cancer (Arlen et al., 1998, Crit. Rev. Immunol. 18:133-138), multiple myeloma (Ozaki et al., 1997, Blood 90:3179-3186; Tsunenari et al., 1997, Blood 90:2437-2444), gastric cancer (Kasprzyk et al., 1992, Cancer Res. 52:2771-2776), B-cell lymphoma (Funakoshi et al., 1996, J. Immunother. Emphasis Tumor Immunol. 19:93-101), leukemia (Zhong et al., 1996, Leuk. Res. 20:581-589), colorectal cancer (Moun et al., 1994, Cancer Res. 54:6160-6166; Velders et al., 1995, Cancer Res. 55:4398-4403), and breast cancer (Shepard et al., 1991, J. Clin. Immunol. 11:117-127). Some therapeutic approaches involve conjugation of naked antibody to a toxin, such as the conjugation of ¹³¹I to anti-CD20 antibodies (e.g., Rituxan™, IDEC Pharmaceuticals Corp.), while others involve co-administration of antibodies and other therapeutic agents, such as Herceptin™ (trastuzumab) with paclitaxel (Genentech, Inc.). For treatment of prostate cancer, for example, 83P5G4 antibodies can be administered in conjunction with radiation, chemotherapy or hormone ablation.

Although 83P5G4 antibody therapy is useful for all stages of cancer, antibody therapy is particularly appropriate in advanced or metastatic cancers. Treatment with the antibody therapy of the invention is indicated for patients who have received one or more rounds of chemotherapy. Alternatively, antibody therapy of the invention is combined with a chemotherapeutic or radiation regimen for patients who have not received chemotherapeutic treatment. Additionally, antibody therapy can enable the use of reduced dosages of concomitant chemotherapy, particularly for patients who do not tolerate the toxicity of the chemotherapeutic agent very well.

It is desirable for some cancer patients to be evaluated for the presence and level of 83P5G4 expression, preferably using immunohistochemical assessments of tumor tissue, quantitative 83P5G4 imaging, or other techniques capable of reliably indicating the presence and degree of 83P5G4 expression. Immunohistochemical analysis of tumor biopsies or surgical specimens is preferred for this purpose. Methods for immunohistochemical analysis of tumor tissues are well-known in the art.

Anti-83P5G4 monoclonal antibodies useful in treating prostate and other cancers include those that are capable of initiating a potent immune response against the tumor or those that are directly cytotoxic. In this regard, anti-83P5G4 monoclonal antibodies (mAbs) can elicit tumor cell lysis by either complement-mediated or antibody-dependent cell cytotoxicity (ADCC) mechanisms, both of which require an intact Fc portion of the immunoglobulin molecule for interaction with effector cell Fc receptor sites on complement proteins. In addition, anti-83P5G4 mAbs that exert a direct biological effect on tumor growth are useful in the practice of the invention. Mechanisms by which directly cytotoxic mAbs act include inhibition of cell growth, modulation of cellular differentiation, modulation of tumor angiogenesis factor profiles, and the induction of apoptosis. The mechanism(s) by which a particular anti-83P5G4 mAb exerts an anti-tumor effect is evaluated using any number of in vitro assays designed to determine cell death such as ADCC, ADMMC, complement-mediated cell lysis, and so forth, as is generally known in the art.

In some patients, the use of murine or other non-human monoclonal antibodies, or human/mouse chimeric mAbs can induce moderate to strong immune responses against the non-human antibody. This can result in clearance of the antibody from circulation and reduced efficacy. In the most severe cases, such an immune response can lead to the extensive formation of immune complexes that, potentially, can cause renal failure. Accordingly, preferred monoclonal antibodies used in the practice of the therapeutic methods of the invention are those that are either fully human or humanized and that bind specifically to the target 83P5G4 antigen with high affinity but exhibit low or no antigenicity in the patient.

Therapeutic methods of the invention contemplate the administration of single anti-83P5G4 mAbs as well as combinations, or cocktails, of different mAbs. Such mAb cocktails can have certain advantages inasmuch as they contain mAbs that target different epitopes, exploit different effector mechanisms or combine directly cytotoxic mAbs with mAbs that rely on immune effector functionality. Such mAbs in combination can exhibit synergistic therapeutic effects. In addition, the administration of anti-83P5G4 mAbs can be combined with other therapeutic agents, including but not limited to various chemotherapeutic agents, androgen-blockers, and immune modulators (e.g., IL-2, GM-CSF). The anti-83P5G4 mAbs are administered in their “naked” or unconjugated form, or can have therapeutic agents conjugated to them.

The anti-83P5G4 antibody formulations are administered via any route capable of delivering the antibodies to the tumor site. Routes of administration include, but are not limited to, intravenous, intraperitoneal, intramuscular, intratumor, intradermnal, and the like. Treatment generally involves the repeated administration of the anti-83P5G4 antibody preparation via an acceptable route of administration such as intravenous injection (IV), typically at a dose in the range of about 0.1 to about 10 mg/kg body weight. Doses in the range of 10-500 mg mAb per week are effective and well tolerated.

Based on clinical experience with the Herceptin mAb in the treatment of metastatic breast cancer, an initial loading dose of approximately 4 mg/kg patient body weight IV, followed by weekly doses of about 2 mg/kg IV of the anti- 83P5G4 mAb preparation represents an acceptable dosing regimen. Preferably, the initial loading dose is administered as a 90 minute or longer infusion. The periodic maintenance dose is administered as a 30 minute or longer infusion, provided the initial dose was well tolerated. However, as appreciated by one of skill in the art, various factors can influence the ideal dose regimen in a particular case. Such factors include, for example, the binding affinity and half life of the Ab or mAbs used, the degree of 83P5G4 expression in the patient, the extent of circulating shed 83P5G4 antigen, the desired steady-state antibody concentration level, frequency of treatment, and the influence of chemotherapeutic agents used in combination with the treatment method of the invention, as well as the health status of a particular patient.

Optionally, patients should be evaluated for the levels of 83P5G4 in a given sample (e.g. the levels of circulating 83P5G4 antigen and/or 83P5G4-expressing cells) in order to assist in the determination of the most effective dosing regimen and related factors. Such evaluations are also be used for monitoring purposes throughout therapy, and are useful to gauge therapeutic success in combination with evaluating other parameters (such as serum PSA levels in prostate cancer therapy).

Inhibition of 83P5G4 Protein Function

The invention includes various methods and compositions for inhibiting the binding of 83P5G4 to its binding partner or its association with other protein(s) as well as methods for inhibiting 83P5G4 function.

Inhibition of 83P5G4 With Intracellular Antibodies

In one approach, recombinant vectors encoding single chain antibodies that specifically bind to 83P5G4 are introduced into 83P5G4-expressing cells via gene transfer technologies. Accordingly, the encoded single chain anti-83P5G4 antibody is expressed intracellularly, binds to 83P5G4 protein, and thereby inhibits its function. Methods for engineering such intracellular single chain antibodies are well-known. Such intracellular antibodies, also known as “intrabodies”, are specifically targeted to a particular compartment within the cell, providing control over where the inhibitory activity of the treatment will be focused. This technology has been successfully applied in the art (for review, see Richardson and Marasco, 1995, TIBTECH vol. 13). Intrabodies have been shown to virtually eliminate the expression of otherwise abundant cell surface receptors. See, for example, Richardson et al., 1995, Proc. Natl. Acad. Sci. USA 92:3137-3141; Beerli et al., 1994, J. Biol. Chem. 289:23931-23936; Deshane et al., 1994, Gene Ther. 1:332-337.

Single chain antibodies comprise the variable domains of the heavy and light chain joined by a flexible linker polypeptide, and are expressed as a single polypeptide. Optionally, single chain antibodies are expressed as a single chain variable region fragment joined to the light chain constant region. Well-known intracellular trafficking signals are engineered into recombinant polynucleotide vectors encoding such single chain antibodies in order to precisely target the expressed intrabody to the desired intracellular compartment. For example, intrabodies targeted to the endoplasmic reticulum (ER) are engineered to incorporate a leader peptide and, optionally, a C-terminal ER retention signal, such as the KDEL amino acid motif. Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

In one embodiment, intrabodies are used to capture 83P5G4 in the nucleus, thereby preventing its activity within the nucleus. Nuclear targeting signals are engineered into such 83P5G4 intrabodies in order to achieve the desired targeting. Such 83P5G4 intrabodies are designed to bind specifically to a particular 83P5G4 domain. In another embodiment, cytosolic intrabodies that specifically bind to the 83P5G4 protein are used to prevent 83P5G4 from gaining access to the nucleus, thereby preventing it from exerting any biological activity within the nucleus (e.g., preventing 83P5G4 from forming transcription complexes with other factors).

In order to specifically direct the expression of such intrabodies to particular cells, the transcription of the intrabody is placed under the regulatory control of an appropriate tumor-specific promoter and/or enhancer. In order to target intrabody expression specifically to prostate, for example, the PSA promoter and/or promoter/enhancer can be utilized (See, for example, U.S. Pat. No. 5,919,652 issued Jul. 6, 1999).

Inhibition of 83P5G4 With Recombinant Proteins

In another approach, recombinant molecules that bind to 83P5G4 thereby prevent or inhibit 83P5G4 from accessing/binding to its binding partner(s) or associating with other protein(s) are used to inhibit 83P5G4 function. Such recombinant molecules can, for example, contain the reactive part(s) of an 83P5G4 specific antibody molecule. In a particular embodiment, the 83P5G4 binding domain of an 83P5G4 binding partner is engineered into a dimeric fusion protein comprising two 83P5G4 ligand binding domains linked to the Fc portion of a human IgG, such as human IgG1. Such IgG portion can contain, for example, the C_{H}2 and C_{H}3 domains and the lunge region, but not the C_{H}1 domain. Such dimeric fusion proteins are administered in soluble form to patients suffering from a cancer associated with the expression of 83P5G4, where the dimeric fusion protein specifically binds to 83P5G4 thereby blocking 83P5G4 interaction with a binding partner. Such dimeric fusion proteins are further combined into multimeric proteins using known antibody linking technologies.

Inhibition of 83P5G4 Transcription or Translation

The invention also provides various methods and compositions for inhibiting the transcription of the 83P5G4 gene. Similarly, the invention also provides methods and compositions for inhibiting the translation of 83P5G4 mRNA into protein.

In one approach, a method of inhibiting the transcription of the 83P5G4 gene comprises contacting the 83P5G4 gene with an 83P5G4 antisense polynucleotide. In another approach, a method of inhibiting 83P5G4 mRNA translation comprises contacting the 83P5G4 mRNA with an antisense polynucleotide. In another approach, an 83P5G4 specific ribozyme is used to cleave the 83P5G4 message, thereby inhibiting translation. Such antisense and ribozyme based methods can also be directed to the regulatory regions of the 83P5G4 gene, such as the 83P5G4 promoter and/or enhancer elements. Similarly, proteins capable of inhibiting an 83P5G4 gene transcription factor are used to inhibit 83P5G4 mRNA transcription. The various polynucleotides and compositions useful in the aforementioned methods have been described above. The use of antisense and ribozyme molecules to inhibit transcription and translation is well-known in the art.

Other factors that inhibit the transcription of 83P5G4 through interfering with 83P5G4 transcriptional activation are also useful to treat cancers expressing 83P5G4. Similarly, factors that interfere with 83P5G4 processing are useful to treat cancers that express 83P5G4. Cancer treatment methods utilizing such factors are also within the scope of the invention.

General Considerations for Therapeutic Strategies

Gene transfer and gene therapy technologies can be used to deliver therapeutic polynucleotide molecules to tumor cells synthesizing 83P5G4 (i.e., antisense, ribozyme, polynucleotides encoding intrabodies and other 83P5G4 inhibitory molecules). A number of gene therapy approaches are known in the art. Recombinant vectors encoding 83P5G4 antisense polynucleotides, ribozymes, factors capable of interfering with 83P5G4 transcription, and so forth, can be delivered to target tumor cells using such gene therapy approaches.

The above therapeutic approaches can be combined with any one of a wide variety of surgical, chemotherapy or radiation therapy regimens. These therapeutic approaches can enable the use of reduced dosages of chemotherapy and/or less frequent administration, an advantage for all patients and particularly for those that do not tolerate the toxicity of the chemotherapeutic agent well.

The anti-tumor activity of a particular composition (e.g., antisense, ribozyme, intrabody), or a combination of such compositions, can be evaluated using various in vitro and in vivo assay systems. In vitro assays for evaluating therapeutic activity include cell growth assays, soft agar assays and other assays indicative of tumor promoting activity, binding assays capable of determining the extent to which a therapeutic composition will inhibit the binding of 83P5G4 to a binding partner, etc.

In vivo, the effect of an 83P5G4 therapeutic composition can be evaluated in a suitable animal model. For example, xenogenic prostate cancer models wherein human prostate cancer explants or passaged xenograft tissues are introduced into immune compromised animals, such as nude or SCID mice, are appropriate in relation to prostate cancer and have been described (Klein et al., 1997, Nature Medicine 3:402-408). For example, PCT Patent Application WO98/16628, Sawyers et al., published Apr. 23, 1998, describes various xenograft models of human prostate cancer capable of recapitulating the development of primary tumors, micrometastasis, and the formation of osteoblastic metastases characteristic of late stage disease. Efficacy can be predicted using assays that measure inhibition of tumor formation, tumor regression or metastasis, and the like. See, also, the Examples below.

In vivo assays that evaluate the promotion of apoptosis are useful in evaluating therapeutic compositions. In one embodiment, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980).

Therapeutic formulations can be solubilized and administered via any route capable of delivering the therapeutic composition to the tumor site. Potentially effective routes of administration include, but are not limited to, intravenous, parenteral, intraperitoneal, intramuscular, intratumor, intradermal, intraorgan, orthotopic, and the like. A preferred formulation for intravenous injection comprises the therapeutic composition in a solution of preserved bacteriostatic water, sterile unpreserved water, and/or diluted in polyvinylchloride or polyethylene bags containing 0.9% sterile Sodium Chloride for Injection, USP. Therapeutic protein preparations can be lyophilized and stored as sterile powders, preferably under vacuum, and then reconstituted in bacteriostatic water containing, for example, benzyl alcohol preservative, or in sterile water prior to injection.

Dosages and administration protocols for the treatment of cancers using the foregoing methods will vary with the method and the target cancer, and will generally depend on a number of other factors appreciated in the art.

### CANCER VACCINES

The invention further provides cancer vaccines comprising an 83P5G4-related protein or fragment as well as DNA based vaccines. In view of the expression of 83P5G4, cancer vaccines are effective at specifically preventing and/or treating 83P5G4-expressing cancers without creating non-specific effects on non-target tissues. The use of a tumor antigen in a vaccine that generates humoral and cell-mediated immune responses as anti-cancer therapy is well-known in the art and has been employed in prostate cancer using human PSMA and rodent PAP immunogens (Hodge et al., 1995, Int. J. Cancer 63:231-237; Fong et al., 1997, J. Immunol. 159:3113-3117).

Such methods can be readily practiced by employing a 83P5G4 protein, or fragment thereof, or an 83P5G4-encoding nucleic acid molecule and recombinant vectors capable of expressing and appropriately presenting the 83P5G4 immunogen (which typically comprises a number of humoral or T cell epitopes). Skilled artisans understand that a wide variety of vaccine systems for delivery of immunoreactive epitopes are known in the art (see, e.g., Heryln et al., Ann Med 1999 Feb; 31(1):66-78; Maruyama et al., Cancer Immunol Immunother 2000 Jun; 49(3):123-32) Briefly, such techniques consist of methods of generating an immune response (e.g. a humoral and/or cell-mediated response) in a mammal comprising the steps of exposing the mammal's immune system to an immunoreactive epitope (e.g. an epitope present in the 83P5G4 protein shown in SEQ ID NO: 2) so that the mammal generates an immune response that is specific for that epitope (e.g. generates antibodies that specifically recognize that epitope). In a preferred method, the 83P5G4 immunogen contains a biological motif. In a highly preferred embodiment, the 83P5G4 immunogen contains one or more amino acid sequences identified using one of the pertinent analytical techniques well-known in the art such as the sequences shown in Tables IV-XVII or a peptide of 8, 9, 10 or 11 amino acids specified by a motif of Table IIIA and IIIB.

A wide variety of methods for generating an immune response in a mammal are well-known in the art (for example as the first step in the generation of hybridomas). Methods of generating an immune response in a mammal comprise exposing the mammal's immune system to an immunogenic epitope on a protein (e.g. the 83P5G4 protein of SEQ ID NO: 2) so that an immune response is generated. A typical embodiment consists of a method for generating an immune response to 83P5G4 in a host, by contacting the host with a sufficient amount of 83P5G4 or a B cell or cytotoxic T-cell eliciting epitope or analog thereof; and at least one periodic interval thereafter contacting the host with additional 83P5G4 or a B cell or cytotoxic T-cell eliciting epitope or analog thereof. A specific embodiment consists of a method of generating an immune response against an 83P5G4 protein or a multiepitopic peptide comprising administering 83P5G4 immunogen (e.g. the 83P5G4 protein or a peptide fragment thereof, an 83P5G4 fusion protein or analog etc.) in a vaccine preparation to humans or animals. Typically, such vaccine preparations further contain a suitable adjuvant (see, e.g., U.S. Pat. No. 6,146,635) or a universal epitope such as a PADRE™ peptide (Epimmune Inc., San Diego, Calif.). See, e.g., Alexander et al., J. Immunol. 2000 164(3); 164(3): 1625-1633; Alexander et al., Immunity 1994 1(9): 751-761 and Alexander et al., Immunol. Res. 1998 18(2): 79-92. A variation on these methods comprises a method of generating an immune response in an individual against an 83P5G4 immunogen by administering in vivo to muscle or skin of the individual's body a genetic vaccine facilitator such as one selected from the group consisting of: anionic lipids; saponins; lectins; estrogenic compounds; hydroxylated lower alkyls; dimethyl sulfoxide; and urea; and a DNA molecule that is dissociated from an infectious agent and comprises a DNA sequence that encodes the 83P5G4 immunogen, the DNA sequence operatively linked to regulatory sequences which control the expression of the DNA sequence; wherein the DNA molecule is taken up by cells, the DNA sequence is expressed in the cells and an immune response is generated against the immunogen. (see, e.g., U.S. Pat. No. 5,962,428).

In an example of a method for generating an immune response, viral gene delivery systems are used to deliver an 83P5G4-encoding nucleic acid molecule. Various viral gene delivery systems that can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8:658-663). Non-viral delivery systems can also be employed by using naked DNA encoding a 83P5G4 protein or fragment thereof introduced into the patient (e.g., intramuscularly or intradermally) to induce an anti-tumor response. In one embodiment, the full-length human 83P5G4 cDNA is employed. In another embodiment, 83P5G4 nucleic acid molecules encoding specific cytotoxic T lymphocyte (CTL) epitopes can be employed. CTL epitopes can be determined using specific algorithms to identify peptides within a 83P5G4 protein that are capable of optimally binding to specified HLA alleles (e.g., Epimer, Brown University; and BIMAS, http://bimas.dcrt.nih.gov/.

Various ex vivo strategies can also be employed. One approach involves the use of antigen presenting cells (APCs) such as dendritic cells that present 83P5G4 antigen to a patient's immune system Dendritic cells express MHC class I and II molecules, B7 co-stimulator, and IL-12, and are thus highly specialized antigen presenting cells. In prostate cancer, autologous dendritic cells pulsed with peptides of the prostate-specific membrane antigen (PSMA) are being used in a Phase I clinical trial to stimulate prostate cancer patients' immune systems (Tjoa et al., 1996, Prostate 28:65-69; Murphy et al., 1996, Prostate 29:371-380). Thus, dendritic cells can be used to present 83P5G4 peptides to T cells in the context of MHC class I or II molecules. In one embodiment, autologous dendritic cells are pulsed with 83P5G4 peptides capable of binding to MHC class I and/or class II molecules. In another embodiment, dendritic cells are pulsed with the complete 83P5G4 protein. Yet another embodiment involves engineering the overexpression of the 83P5G4 gene in dendritic cells using various implementing vectors known in the art, such as adenovirus (Arthur et al., 1997, Cancer Gene Ther. 4:17-25), retrovirus (Henderson et al., 1996, Cancer Res. 56:3763-3770), lentivirus, adeno-associated virus, DNA transfection (Ribas et al., 1997, Cancer Res. 57:2865-2869), or tumor-derived RNA transfection (Ashley et al., 1997, J. Exp. Med. 186:1177-1182). Cells expressing 83P5G4 can also be engineered to express immune modulators, such as GM-CSF, and used as immunizing agents.

Anti-idiotypic anti-83P5G4 antibodies can also be used in anti-cancer therapy as a vaccine for inducing an immune response to cells expressing an 83P5G4 protein. Specifically, the generation of anti-idiotypic antibodies is well-known in the art and can readily be adapted to generate anti-idiotypic anti-83P5G4 antibodies that mimic an epitope on a 83P5G4 protein (see, for example, Wagner et al., 1997, Hybridoma 16:33-40; Foon et al., 1995, J. Clin. Invest. 96:334-342; Herlyn et al., 1996, Cancer Immunol. Immunother. 43:65-76). Such an anti-idiotypic antibody can be used in cancer vaccine strategies.

Genetic immunization methods can be employed to generate prophylactic or therapeutic humoral and cellular immune responses directed against cancer cells expressing 83P5G4. Constructs comprising DNA encoding an 83P5G4-related protein/immunogen and appropriate regulatory sequences can be injected directly into muscle or skin of an individual, such that the cells of the muscle or skin take-up the construct and express the encoded 83P5G4 protein/immunogen. Alternatively, a vaccine comprises an 83P5G4-related protein. Expression of the 83P5G4-related protein immunogen results in the generation of prophylactic or therapeutic humoral and cellular immunity against cells that bear 83P5G4 protein. Various prophylactic and therapeutic genetic immunization techniques known in the art can be used (for review, see information and references published at Internet address www.genweb.com).

### KITS

For use in the diagnostic and therapeutic applications described herein, kits are also within the scope of the invention. Such kits can comprise a carrier that is compartmentalized to receive one or more containers such as vials, tubes, and the like, each of the container(s) comprising one of the separate elements to be used in the method. For example, the container(s) can comprise a probe that is or can be detectably labeled. Such probe can be an antibody or polynucleotide specific for an 83P5G4-related protein or an 83P5G4 gene or message, respectively. Where the kit utilizes nucleic acid hybridization to detect the target nucleic acid, the kit can also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label. The kit can include all or part of the amino acid sequences of FIG. 2 or an analog thereof, or a nucleic acid molecule that encodes such amino acid sequences.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above. p83P5G4-1 has been deposited under the requirements of the Budapest Treaty on Jan. 6, 2000 with the American Type Culture Collection (ATCC), 10801 University Blvd., Manassas, Va. 20110-2209 USA, and has been identified as ATCC Accession No. PTA- 1154.

### EXAMPLES

Various aspects of the invention are further described and illustrated by way of the several examples that follow, none of which are intended to limit the scope of the invention.

### Example 1: SSH-Generated Isolation of a cDNA Fragment of the 83P5G4 Gene Materials and Methods

LAPC Xenografts and Human Tissues:

LAPC xenografts were obtained from Dr. Charles Sawyers (UCLA) and generated as described (Klein et al, 1997, Nature Med. 3:402-408; Craft et al., 1999, Cancer Res. 59:5030-5036). Androgen dependent and independent LAPC-4 xenografts LAPC-4 AD and Al, respectively) and LAPC-9 AD and Al xenografts were grown in male SCID mice and were passaged as small tissue chunks in recipient males. LAPC-4 and -9 Al xenografts were derived from LAPC-4 or -9 AD tumors, respectively. To generate the Al xenografts, male mice bearing AD tumors were castrated and maintained for 2-3 months. After the tumors re-grew, the tumors were harvested and passaged in castrated males or in female SCID mice.

Cell Lines:

Human cell lines (e.g., HeLa) were obtained from the ATCC and were maintained in DMEM with 5% fetal calf serum.

RNA Isolation:

Tumor tissue and cell lines were homogenized in Trizol reagent (Life Technologies, Gibco BRL) using 10 ml/ g tissue or 10 ml/ 10⁸ cells to isolate total RNA. Poly A RNA was purified from total RNA using Qiagen's Oligotex mRNA Mini and Midi kits. Total and mRNA were quantified by spectrophotometric analysis (O.D. 260/280 nm) and analyzed by gel electrophoresis.

Oligonucleotides:

The following HPLC purified oligonucleotides were used.

| | |
|---|---|
| DPNCDN (cDNA synthesis primer): | |
| 5′TTTTGATCAAGCTT303′ | (SEQ ID NO:7) |
| Adaptor 1: | |
| 5′CTAATACGACTCACTATAGGGCTCGAGCGGCCGCCCGGGCAG3′ | (SEQ ID NO:8) |
| 3′GGCCCGTCCTAG5′ | (SEQ ID NO:9) |
| Adaptor 2: | |
| 5′GTAATACGACTCACTATAGGGCAGCGTGGTCGCGGCCGAG3′ | (SEQ ID NO:10) |
| 3′CGGCTCCTAG5′ | (SEQ ID NO:11) |
| PCR primer 1: | |
| 5′CTAATACGACTCACTATAGGGC3′ | (SEQ ID NO:12) |
| Nested primer (NP)1: | |
| 5′TCGAGCGGCCGCCCGGGCAGGA3′ | (SEQ ID NO:13) |
| Nested primer (NP)2: | |
| 5′AGCGTGGTCGCGGCCGAGGA3′ | (SEQ ID NO:14) |

Suppression Subtractive Hybridization:

Suppression Subtractive Hybridization (SSH) was used to identify cDNAs corresponding to genes that may be differentially expressed in prostate cancer. The SSH reaction utilized cDNA from two LAPC-4 AD xenografts. Specifically, mice that harbored LAPC-4 AD xenografts were castrated when the tumors reached a size of 1 cm in diameter. The tumors stopped growing and temporarily stopped producing the androgen dependent protein PSA. Seven to fourteen days post-castration, PSA levels were detectable again in the blood of the mice. Eventually the tumors develop an AI phenotype and start growing again in the castrated males. Tumors were harvested at different time points after castration to identify genes that are turned on or off during the transition to androgen independence.

The 83P5G4 SSH sequence was identified from a subtraction where cDNA derived from an LAPC-4 AD tumor, 3 days post-castration, was subtracted from cDNA derived from an LAPC-4 AD tumor grown in an intact male. The LAPC-4 AD xenograft tumor grown in an intact male was used as the source of the “tester” cDNA, while the cDNA from the LAPC-4 AD tumor, 3 days post-castration, was used as the source of the “driver” cDNA.

Double stranded cDNAs corresponding to tester and driver cDNAs were synthesized from 2 μg of poly(A)³⁰ RNA isolated from the relevant xenograft tissue, as described above, using CLONTECH's PCR-Select cDNA Subtraction Kit and 1 ng of oligonucleotide DPNCDN as primer. First- and second-strand synthesis were carried out as described in the Kit's user manual protocol (CLONTECH Protocol No. PT1117-1, Catalog No. K1804-1). The resulting cDNA was digested with Dpn II for 3 hrs. at 37° C. Digested cDNA was extracted with phenol/chloroform (1:1) and ethanol precipitated.

Driver cDNA was generated by combining in a 1:1 ratio Dpn II digested cDNA from the relevant xenograft source (see above) with a mix of digested cDNAs derived from the human cell lines HeLa, 293, A431, Colo205, and mouse liver.

Tester cDNA was generated by diluting 1 μl of Dpn II digested cDNA from the relevant xenograft source (see above) (400 ng) in 5 μl of water. The diluted cDNA (2 μl, 160 ng) was then ligated to 2 μl of Adaptor 1 and Adaptor 2 (10 μM), in separate ligation reactions, in a total volume of 10 μl at 16° C. overnight, using 400 u of T4 DNA ligase (CLONTECH). Ligation was terminated with 1 μl of 0.2 M EDTA and heating at 72° C. for 5 min.

The first hybridization was performed by adding 1.5 μl (600 ng) of driver cDNA to each of two tubes containing 1.5 μl (20 ng) Adaptor 1- and Adaptor 2-ligated tester cDNA. In a final volume of 4 μl, the samples were overlaid with mineral oil, denatured in an MJ Research thermal cycler at 98° C. for 1.5 minutes, and then were allowed to hybridize for 8 hrs at 68° C. The two hybridizations were then mixed together with an additional 1 p1 of fresh denatured driver cDNA and were allowed to hybridize overnight at 68° C. The second hybridization was then diluted in 200 μl of 20 mM Hepes, pH 8.3, 50 mM NaCl, 0.2 mM EDTA, heated at 70° C. for 7 min. and stored at −20° C.

PCR Amplification, Cloning and Sequencing of Gene Fragments Generated from SSH:

To amplify gene fragments resulting from SSH reactions, two PCR amplifications were performed. In the primary PCR reaction 1 p1 of the diluted final hybridization mix was added to 1 μl of PCR primer 1 (10 μM), 0.5 μl dNTP mix (10 μM), 2.5 μl 10×reaction buffer (CLONTECH) and 0.5 μl 50×Advantage cDNA polymerase Mix (CLONTECH) in a final volume of 25 μl. PCR 1 was conducted using the following conditions: 75° C. for 5 min., 94° C. for 25 sec., then 27 cycles of 94° C. for 10 sec, 66° C. for 30 sec, 72° C. for 1.5 min. Five separate primary PCR reactions were performed for each experiment. The products were pooled and diluted 1:10 with water. For the secondary PCR reaction, 1 μl from the pooled and diluted primary PCR reaction was added to the same reaction mix as used for PCR 1, except that primers NP1 and NP2 (10 μM) were used instead of PCR primer 1. PCR 2 was performed using 10-12 cycles of 94° C. for 10 sec, 68° C. for 30 sec, and 72° C. for 1.5 minutes. The PCR products were analyzed using 2% agarose gel electrophoresis.

The PCR products were inserted into pCR2.1 using the T/A vector cloning kit (Invitrogen). Transformed E. coli were subjected to blue/white and ampicillin selection. White colonies were picked and arrayed into 96 well plates and were grown in liquid culture overnight. To identify inserts, PCR amplification was performed on 1 ml of bacterial culture using the conditions of PCR1 and NP1 and NP2 as primers. PCR products were analyzed using 2% agarose gel electrophoresis.

Bacterial clones were stored in 20% glycerol in a 96 well format. Plasmid DNA was prepared, sequenced, and subjected to nucleic acid homology searches of the GenBank, dBest, and NCI-CGAP databases.

RT-PCR Expression Analysis:

First strand cDNAs can be generated from 1 μg of mRNA with oligo (dT) 12-18 priming using the Gibco-BRL Superscript Preamplification system. The manufacturer's protocol was used which included incubation for 50 min at 42° C. with reverse transcriptase followed by RNAse H treatment at 37° C. for 20 min. After completing the reaction, the volume can be increased to 200 μl with water prior to normalization. First strand cDNAs from 16 different normal human tissues can be obtained from Clontech.

Normalization of the first strand cDNAs from multiple tissues was performed by using the primers 5′atatcgccgcgctcgtcgtcgacaa3′ (SEQ ID NO: 15) and 5′agccacacgcagctcattgtagaagg 3′ (SEQ ID NO: 16) to amplify β-actin. First strand cDNA (5 μl) were amplified in a total volume of 50 μl containing 0.4 μM primers, 0.2 μM each dNTPs, 1×PCR buffer (Clontech, 10 mM Tris-HCL, 1.5 mM MgCl₂, 50 mM KCl, pH8.3) and 1×Klentaq DNA polymerase (Clontech). Five μl of the PCR reaction can be removed at 18, 20, and 22 cycles and used for agarose gel electrophoresis. PCR was performed using an MJ Research thermal cycler under the following conditions: Initial denaturation can be at 94° C. for 15 sec, followed by a 18, 20, and 22 cycles of 94° C. for 15, 65° C. for 2 min, 72° C. for 5 sec. A final extension at 72° C. was carried out for 2 min. After agarose gel electrophoresis, the band intensities of the 283 b.p. β-actin bands from multiple tissues were compared by visual inspection. Dilution factors for the first strand cDNAs were calculated to result in equal β-actin band intensities in all tissues after 22 cycles of PCR. Three rounds of normalization can be required to achieve equal band intensities in all tissues after 22 cycles of PCR.

To determine expression levels of the 83P5G4 gene, 5 ill of normalized first strand cDNA were analyzed by PCR using 25, 30, and 35 cycles of amplification. Semi quantitative expression analysis can be achieved by comparing the PCR products at cycle numbers that give light band intensities.

In a typical RT-PCR Expression analysis shown in FIG. 10, RT-PCR expression analysis was performed on first strand cDNAs generated using pools of tissues from multiple samples. The cDNAs were subsequently normalized using beta-actin PCR. The highest expression was observed in normal prostate, prostate cancer xenografts, and prostate cancer tissue pools and a lung cancer patient. Lower levels of expression were also observed in bladder, kidney, and colon cancer tissue pools.

Results

Two SSH experiments described in the Materials and Methods, supra, led to the isolation of numerous candidate gene fragment clones (SSH clones). All candidate clones were sequenced and subjected to homology analysis against all sequences in the major public gene and EST databases in order to provide information on the identity of the corresponding gene and to help guide the decision to analyze a particular gene for differential expression. In general, gene fragments that had no homology to any known sequence in any of the searched databases, and thus considered to represent novel genes, as well as gene fragments showing homology to previously sequenced expressed sequence tags (ESTs), were subjected to differential expression analysis by RT-PCR and/or Northern analysis.

One of the SSH clones comprising about 445 b.p. showed significant homology to several testis-derived ESTs and the proteins described below, and was designated 83P5G4.

### Example 2: Full-length Cloning of 83P5G4

A full-length 83P5G4 cDNA clone (clone 1) of 2840 base pairs (b.p.) was cloned from an LAPC-4 AD cDNA library (Lambda ZAP Express, Stratagene) (FIG. 2). The cDNA encodes an open reading frame (ORF) of 730 amino acids, with the codon for the N-terminal methionine occurring at nucleotides 130-132 as shown in FIG. 2. Alternatively, the codon for the N-terminal methionine of the open reading frame may occur at nucleotides 316-318 as shown in FIG. 2, thereby encoding a protein of 668 amino acids. The protein sequence reveals a single nuclear localization signal and is predicted to be nuclear in localization using the PSORT program (http://psort.nibb.ac.jp:8800/form,html). Its calculated molecular weight (MW) 79.4 kDa and its pI is 9.08.

Sequence analysis of 83P5G4 reveals homology to the lethal (2) denticless protein of Drosophila (Kurzik-Dumke et al., 1996, Gene 171:163-170). The two protein sequences are 42% identical and 60% homologous over a 352 amino acid region (FIG. 3). The 83P5G4 amino acid sequence contains 5 predicted WD40 repeat domains, a nuclear localization signal (residues 199-203), two ser/pro rich regions (44% of amino acids within residues 425 and 520 and 43% of amino acids within residues 608-642), and a leucine zipper domain (residues 577-598). The human denticleless gene, as reported by Mueller and Ziegler (GenBank Accession NM_{—}016448), contains WD-40 repeats and has one amino acid difference when compared to the 83P5G4 protein where 83P5G3 has an alanine at position five and human denticleless has a valine. This homology confirms that 83P5G4 is the human homolog of the drosophila lethal (2) denticleless protein. The drosophila lethal (2) dentceleless protein is a heat-shock protein due to the fact that its expression is regulated by heat (Kurzik-Dumke et al., 1996, Gene 171:163-170) suggesting that 83P5G4 is also a heat-shock protein.

The 83P5G4 cDNA was deposited on January 5, 2000 with the American Type Culture Collection (ATCC; Manassas, Va.) as plasmid p83P5G4-1, and has been assigned Accession No. PTA-1154.

### Example 3: 83P5G4 Gene Expression Analysis

83P5G4 mRNA expression in normal human tissues was analyzed by Northern blotting of two multiple tissue blots (Clontech; Palo Alto, California), comprising a total of 16 different normal human tissues, using labeled 83P5G4 SSH fragment (Example 1) as a probe. RNA samples were quantitatively normalized with a β-actin probe. The results demonstrated expression in all normal tissues tested (FIG. 4). The 83P5G4 gene produces 3 transcripts of 1.8, 2.5 and 4.5 kb. Different tissues express different transcripts. For instance brain is the only tissue that expresses all three transcripts. Liver, skeletal muscle, spleen, prostate and leukocytes only express the 1.8 kb transcript. Lung only expresses the 2.5 kb transcript. Kidney and pancreas express the 1.8 and 2.5 kb transcripts. Thymus, ovary, small intestine and colon express the 1.8 and 4.5 kb transcripts. Heart, placenta and testis express the 2.5 and 4.5 kb transcripts. The highest expression levels in normal tissues are detected in testis.

To analyze 83P5G4 expression in prostate cancer tissues lines, Northern blotting was performed on RNA derived from the LAPC xenografts. The results show very high expression levels of the 2.5 and 4.5 kb transcripts in LAPC-4 AD, LAPC-4 Al, LAPC-9 AD, and LAPC-9 AI. It is unclear whether the different transcripts represent alternatively spliced isoform, or whether they represent unprocessed RNA species. The fact that different tissues express different transcripts suggests that the former is the case. It is possible that 83P5G4 isoforms expressed in the prostate cancer xenografts are the same isoforms that are expressed in testis. These results provide evidence that 83P5G4 is up-regulated in prostate cancer.

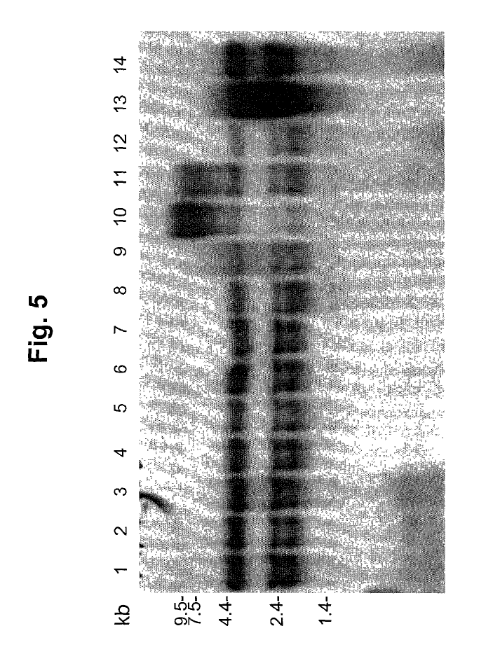

To further analyze 83P5G4 expression in cancer tissues Northern blotting was performed on RNA derived from the LAPC xenografts, and several prostate and non-prostate cancer cell lines. The results show very high expression levels of the 2.5 and 4.5 kb transcripts in LAPC-4 AD, LAPC-4 AI, LAPC-9 AD, LAPC-9 AI (FIG. 4) and LAPC-3 AI (FIG. 5). More detailed analysis of the xenografts shows that 83P5G4 is highly expressed in the xenografts even when grown within the tibia of mice (FIG. 5).

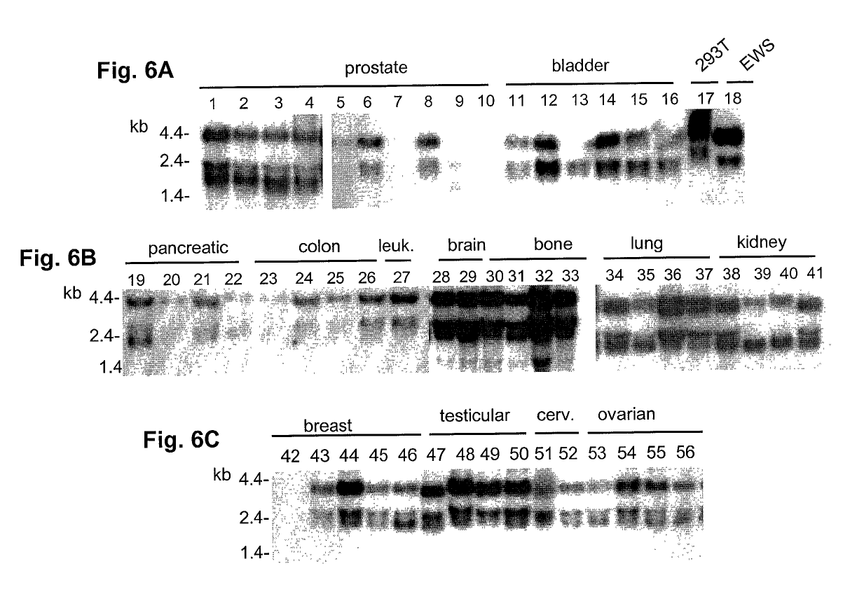

High expression levels of 83P5G4 were detected in several cancer cell lines derived from prostate (DU145, PC-3), bladder (SCABER, TCCSUP, J82), pancreas (PANC-1), brain (PFSK-1, T98G), bone (SK-ES-1, HOS, U2-OS, RD-ES), lung (CALU-1, A427, NCI-H82, NCI-H146), kidney (769-P, A498, CAKI-1, SW839), breast (DU4475), testis (NTERRA-2, NCCIT, TERA-1, TERA-2), and ovary (PA-1, SW626) (FIG. 6). Lower expression levels were also detected in multiple colon, breast, bladder, ovarian and cervical cancer cell lines. Interestingly, in all cases the same two transcripts are detected in these cancer cell lines as are seen in the LAPC xenografts and in testis.

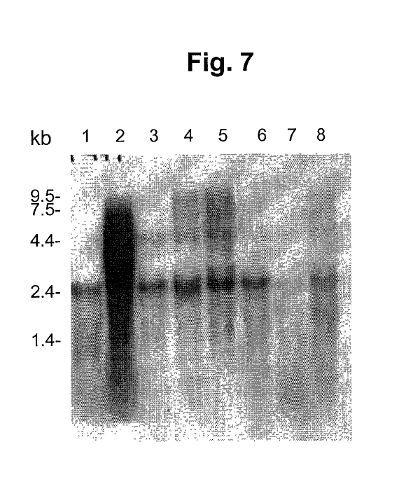

Northern analysis also shows that 83P5G4 is expressed in the normal prostate and prostate tumor tissues derived from prostate cancer patients (FIG. 7). 83P5G4 expression in normal tissues can be further analyzed using a multi-tissue RNA dot blot containing different samples (representing mainly normal tissues as well as a few cancer cell lines).

### Example 4: Generation of 83P5G4 Polyclonal Antibodies

Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. For example, 83P5G4, recombinant bacterial fusion proteins or peptides encoding various regions of the 83P5G4 sequence are used to immunize New Zealand White rabbits. Typically a peptide can be designed from a coding region of 83P5G4. The peptide can be conjugated to keyhole limpet hemocyanin (KLH) and used to immunize a rabbit. Alternatively the immunizing agent may include all or portions of the 83P5G4 protein, analogs or fusion proteins thereof. For example, the 83P5G4 amino acid sequence can be fused to any one of a variety of fusion protein partners that are well-known in the art, such as maltose binding protein, LacZ, thioredoxin or an immunoglobulin constant region (see e.g. Current Protocols In Molecular Biology, Volume 2, Unit 16, Frederick M. Ausubul et al. eds., 1995; Linsley, P. S., Brady, W., Urnes, M., Grosmaire, L., Damle, N., and Ledbetter, L.(1991) J.Exp. Med. 174, 561-566). Other recombinant bacterial proteins include glutathione-S-transferase (GST), and HIS tagged fusion proteins of 83P5G4 that are purified from induced bacteria using the appropriate affinity matrix.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate).

In a typical protocol, rabbits are initially immunized subcutaneously with about 200 μg of fusion protein or peptide conjugated to KLH mixed in complete Freund's adjuvant. Rabbits are then injected subcutaneously every two weeks with 200 μg of immunogen in incomplete Freund's adjuvant. Test bleeds are taken approximately 7-10 days following each immunization and used to monitor the titer of the antiserum by ELISA.

To test serum, such as rabbit serum, for reactivity with 83P5G4 proteins, the full-length 83P5G4 cDNA can be cloned into an expression vector such as one that provides a six His tag at the carboxyl-terminus (pCDNA 3.1 myc-his, Invitrogen). After transfection of the constructs into 293T cells, cell lysates can be probed with anti-His antibody (Santa Cruz Biotechnologies, Santa Cruz, Calif.) and the anti-83P5G4 serum using Western blotting. Alternatively specificity of the antiserum is tested by Western blot and immunoprecipitation analyses using lysates of cells that express 83P5G4. Serum from rabbits immunized with GST or MBP fusion proteins is first semi-purified by removal of anti-GST or anti-MBP antibodies by passage over GST and MBP protein columns respectively. Sera from His-tagged protein and peptide immunized rabbits as well as depleted GST and MBP protein sera are purified by passage over an affinity column composed of the respective immunogen covalently coupled to Affigel matrix (BioRad).

### Example 5: Production of Recombinant 83P5G4 in Bacterial and Mammalian Systems

### BACTERIAL CONSTRUCTS

pGEX Constructs

To express 83P5G4 in bacterial cells, portions of 83P5G4 are fused to the Glutathione S-transferase (GST) gene by cloning into pGEX-6P-1 (Amersham Pharmacia Biotech, NJ). The constructs are made in order to generate recombinant 83P5G4 protein sequences with GST fused at the N-terminus and a six histidine epitope at the C-terminus. The six histidine epitope tag is generated by adding the histidine codons to the cloning primer at the 3′ end of the open reading frame (ORF). A PreScission™ recognition site permits cleavage of the GST tag from 83P5G4-related protein. The ampicillin resistance gene and pBR322 origin permits selection and maintenance of the plasmid in E. coli. For example, the following fragments of 83P5G4 are cloned into pGEX-6P-1: amino acids 1 to 730; amino acids 1 to 150; amino acids 150 to 300; amino acids 300 to 450, and amino acids 450 to 600, 600 to 730, or any 8, 9, 10, 11, 12,13, 14 or 15 contiguous amino acids from 83P5G4 or an analog thereof.

pMAL Constructs

To express 83P5G4 in bacterial cells, all or part of the 83P5G4 nucleic acid sequence are fused to the maltose-binding protein (MBP) gene by cloning into pMAL-c2X and pMAL-p2X (New England Biolabs, MA). The constructs are made to generate recombinant 83P5G4 protein sequences with MBP fused at the N-terminus and a six histidine epitope at the C-terminus. The six histidine epitope tag is generated by adding the histidine codons to the 3′ cloning primer. A Factor Xa recognition site permits cleavage of the GST tag from 83P5G4. The pMAL-c2X and pMAL-p2X vectors are optimized to express the recombinant protein in the cytoplasm or periplasm respectively. Periplasm expression enhances folding of proteins with disulfide bonds. For example, constructs are made in pMAL-c2X and pMAL-p2X that express the following regions of the 83P5G4 protein: amino acids 1 to 730; amino acids 1 to 150; amino acids 150 to 300; amino acids 300 to 450, 450 to 600, or 600 to 730, or any 8, 9, 10, 11, 12,13, 14 or 15 contiguous amino acids from 83P5G4 or an analog thereof.

### MAMMALIAN CONSTRUCTS

To express recombinant 83P5G4, the full or partial length 83P5G4 cDNA can be cloned into any one of a variety of expression vectors known in the art. The constructs can be transfected into any one of a wide variety of mammalian cells such as 293T cells. Transfected 293T cell lysates can be probed with the anti-83P5G4 polyclonal serum, described in Example 4 above, in a Western blot.

The 83P5G4 genes can also be subcloned into the retroviral expression vector pSRαMSVtkneo and used to establish 83P5G4-expressing cell lines as follows: The 83P5G4 coding sequence (from translation initiation ATG to the termination codons) is amplified by PCR using ds cDNA template from 83P5G4 cDNA. The PCR product is subcloned into pSRαMSVtkneo via the EcoR1(blunt-ended) and Xba 1 restriction sites on the vector and transformed into DH5α competent cells. Colonies are picked to screen for clones with unique internal restriction sites on the cDNA. The positive clone is confirmed by sequencing of the cDNA insert. The retroviral vectors can thereafter be used for infection and generation of various cell lines using, for example, NIH 3T3, TsuPr1, 293 or rat-1 cells.

Additional illustrative mammalian and bacterial systems are discussed below.

pcDNA4/HisMax-TOPO Constructs

To express 83P5G4 in mammalian cells, the 83P5G4 ORF is cloned into pcDNA4/HisMax-TOPO Version A (cat# K864-20, Invitrogen, Carlsbad, Calif.). Protein expression is driven from the cytomegalovirus (CMV) promoter and the SP163 translational enhancer. The recombinant protein has Xpress™ and six histidine epitopes fused to the N-terminus. The pcDNA4/HisMax-TOPO vector also contains the bovine growth hormone (BGH) polyadenylation signal and transcription termination sequence to enhance mRNA stability along with the SV40 origin for episomal replication and simple vector rescue in cell lines expressing the large T antigen. The Zeocin resistance gene allows for selection of mammalian cells expressing the protein and the ampicillin resistance gene and Co1E1 origin permits selection and maintenance of the plasmid in E. coli.

pcDNA3.1/MycHis Constructs

To express 83P5G4 in mammalian cells, the ORF with consensus Kozak translation initiation site is cloned into pcDNA3.1/MycHis_Version A (Invitrogen, Carlsbad, Calif.). Protein expression is driven from the cytomegalovirus (CMV) promoter. The recombinant protein has the myc epitope and six histidines fused to the C-terminus. The pcDNA3.1/MycHis vector also contains the bovine growth hormone (BGH) polyadenylation signal and transcription termination sequence to enhance mRNA stability, along with the SV40 origin for episomal replication and simple vector rescue in cell lines expressing the large T antigen. The Neomycin resistance gene can be used, as it allows for selection of mammalian cells expressing the protein and the ampicillin resistance gene and Co1E1 origin permits selection and maintenance of the plasmid in E. coli.

pcDNA3.1CT-GFP-TOPO Construct

To express 83P5G4 in mammalian cells and to allow detection of the recombinant protein using fluorescence, the ORF with consensus Kozak translation initiation site is cloned into pcDNA3.1CT-GFP-TOPO (Invitrogen, Calif.). Protein expression is driven from the cytomegalovirus (CMV) promoter. The recombinant protein has the Green Fluorescent Protein (GFP) fused to the C-terminus facilitating non-invasive, in vivo detection and cell biology studies. The pcDNA3.1/MycHis vector also contains the bovine growth hormone (BGH) polyadenylation signal and transcription termination sequence to enhance mRNA stability along with the SV40 origin for episomal replication and simple vector rescue in cell lines expressing the large T antigen. The Neomycin resistance gene allows for selection of mammalian cells that express the protein, and the ampicillin resistance gene and Co1E1 origin permits selection and maintenance of the plasmid in E. coli. An additional construct with a N-terminal GFP fusion is made in pcDNA3.1NT-GFP-TOPO spanning the entire length of the 83P5G4 protein.

pAPtag

The 83P5G4 ORF is cloned into pAPtag-5 (GenHunter Corp. Nashville, Tenn.). This construct generates an alkaline phosphatase fusion at the C-terminus of the 83P5G4 protein while fusing the IgGK signal sequence to N-terminus. The resulting recombinant 83P5G4 protein is optimized for secretion into the media of transfected mammalian cells and can be used to identify proteins such as ligands or receptors that interact with the 83P5G4 protein. Protein expression is driven from the CMV promoter and the recombinant protein also contains myc and six histidines fused to the C-terminus of alkaline phosphatase. The Zeocin resistance gene allows for selection of mammalian cells expressing the protein and the ampicillin resistance gene permits selection of the plasmid in E. coli.

ptag5

The 83P5G4 ORF is also cloned into pTag-5. This vector is similar to pAPtag but without the alkaline phosphatase fusion. This construct generates an immunoglobulin G1 Fc fusion at the C-terminus of the 83P5G4 protein while fusing the IgGK signal sequence to the N-terminus. The resulting recombinant 83P5G4 protein is optimized for secretion into the media of transfected mammalian cells, and can be used to identify proteins such as ligands or receptors that interact with the 83P5G4 protein. Protein expression is driven from the CMV promoter and the recombinant protein also contains myc and six histidines fused to the C-terminus of alkaline phosphatase. The Zeocin resistance gene allows for selection of mammalian cells expressing the protein, and the ampicillin resistance gene permits selection of the plasmid in E. coli.

psecFc

The 83P5G4 ORF is also cloned into psecFc. The psecFc vector was assembled by cloning immunoglobulin G1 Fc (hinge, CH2, CH3 regions) into pSecTag2 (Invitrogen, California). This construct generates an immunoglobulin G1 Fc fusion at the C-terminus of the 83P5G4 protein, while fusing the IgGK signal sequence to N-terminus. The resulting recombinant 83P5G4 protein is optimized for secretion into the media of transfected mammalian cells, and can be used to identify proteins such as ligands or receptors that interact with the 83P5G4 protein. Protein expression is driven from the CMV promoter and the recombinant protein also contains myc and six histidines fused to the C-terminus of alkaline phosphatase. The Zeocin resistance gene allows for selection of mammalian cells that express the protein, and the ampicillin resistance gene permits selection of the plasmid in E. coli.

pSRα Constructs

To generate mammalian cell lines that express 83P5G4 constitutively, the ORF is cloned into pSRα constructs. Amphotropic and ecotropic retroviruses are generated by transfection of pSRα constructs into the 293T-10A1 packaging line or co-transfection of pSRα and a helper plasmid (ψ˜) in the 293 cells, respectively. The retrovirus can be used to infect a variety of mammalian cell lines, resulting in the integration of the cloned gene, 83P5G4, into the host cell-lines. Protein expression is driven from a long terminal repeat (LTR). The Neomycin resistance gene allows for selection of mammalian cells that express the protein, and the ampicillin resistance gene and Co1E1 origin permit selection and maintenance of the plasmid in E. coli.

An additional pSRα construct was made that fused the FLAG tag to the C-terminus to allow detection using anti-FLAG antibodies. The FLAG sequence 5′ gat tac aag gat gac gac gat aag 3′ (SEQ ID NO: 6) were added to cloning primer at the 3′ end of the ORF.

Additional pSRα constructs are made to produce both N-terminal and C-terminal GFP and myc6/HIS fusion proteins of the full-length 83P5G4 protein.

### Example 6: Production of Recombinant 83P5G4 in a Baculovirus System

To generate a recombinant 83P5G4 protein in a baculovirus expression system, 83P5G4 cDNA is cloned into the baculovirus transfer vector pBlueBac 4.5 (Invitrogen), which provides a His-tag at the N-terminus Specifically, pBlueBac--83P5G4 is co-transfected with helper plasmid pBac-N-Blue (Invitrogen) into SF9 (Spodoptera frugiperda) insect cells to generate recombinant baculovirus (see Invitrogen instruction manual for details). Baculovirus is then collected from cell supernatant and purified by plaque assay.

Recombinant 83P5G4 protein is then generated by infection of HighFive insect cells (Invitrogen) with the purified baculovirus. Recombinant 83P5G4 protein can be detected using anti-83P5G4 antibody. 83P5G4 protein can be purified and used in various cell-based assays or as immunogen to generate polyclonal and monoclonal antibodies specific for 83P5G4.

### Example 7: Chromosomal Mapping of the 83P5G4 Gene

The chromosomal localization of 83P5G4 is listed in the NCBI Map Viewer, http://www.ncbi.nlm.nih.gov/genome/sts/sts.cgi?uid=91173. Mapping was determined using the GeneBridge 4 Human/Hamster radiation hybrid (RH) panel (Walter et al., 1994, Nat. Genetics 7:22)(Research Genetics, Huntsville Ala.). 83P5G4 maps to chromosome 1q31-q32.l between D1S491-D1S474.

### Example 8: Identification of signaling pathways regulated by 83P5G4.

As previously mentioned, WD40-motif containing proteins transmit signals from the cell surface to the nucleus. These proteins function by physically interacting with a variety of signaling molecules and TRP-containing proteins. For example, by using immunoprecipitation and Western blotting techniques, proteins are identified that associate with 83P5G4 and mediate signaling events. These techniques permit one to study several pathways known to play a role in cancer biology, including phospholipid pathways such as P13K, AKT, etc, as well as mitogenic/survival cascades such as ERK, p38, etc (Cell Growth Differ. 2000,11:279; J Biol. Chem. 1999, 274:801; Oncogene 2000, 19:3003.). Signaling pathways activated by 83P5G4 are mapped and used for the identification and validation of therapeutic targets in the 83P5G4 pathway. When 83P5G4 mediates signaling events, 83P5G4 is used as a target for diagnostic, preventative and therapeutic purposes.

### Example 9: Generation of 83P5G4 Monoclonal Antibodies

To generate MAbs to 83P5G4, mice are immunized intraperitoneally with 10-50 μg of protein immunogen mixed in complete Freund's adjuvant. Protein immunogens include peptides, recombinant 83P5G4 proteins, and, mammalian expressed human IgG FC fusion proteins. Mice are then subsequently immunized every 2-4 weeks with 10-50 μg of antigen mixed in Freund's incomplete adjuvant. Alternatively, Ribi adjuvant is used for initial immunizations. In addition, a DNA-based immunization protocol is used in which a mammalian expression vector used to immunize mice by direct injection of the plasmid DNA. For example, a pCDNA 3.1 encoding 83P5G4 cDNA alone or as an IgG FC fusion is used. This protocol is used alone or in combination with protein immunogens. Test bleeds are taken 7-10 days following immunization to monitor titer and specificity of the immune response. Once appropriate reactivity and specificity is obtained as determined by ELISA, Western blotting, and immunoprecipitation analyses, fusion and hybridoma generation is then carried with established procedures well-known in the art (Harlow and Lane, 1988).

In an illustrative method for generating 83P5G4 monoclonal antibodies, a glutathione-S-transferase (GST) fusion protein encompassing an 83P5G4 protein is synthesized and used as immunogen. Balb C mice are initially immunized intraperitoneally with 200 jig of the GST-83P5G4 fusion protein mixed in complete Freund's adjuvant. Mice are subsequently immunized every two weeks with 75 μg of GST-83P5G4 protein mixed in Freund's incomplete adjuvant for a total of three immunizations. Reactivity of serum from immunized mice to full-length 83P5G4 protein is monitored by ELISA using a partially purified preparation of HIS-tagged 83P5G4 protein expressed from 293T cells (Example 5). Mice showing the strongest reactivity are rested for three weeks and given a final injection of fusion protein in PBS and then sacrificed four days later. The spleens of the sacrificed mice are then harvested and fused to SPO/2 myeloma cells using standard procedures (Harlow and Lane, 1988). Supernatants from growth wells following HAT selection are screened by ELISA and Western blot to identify 83P5G4 specific antibody-producing clones.

The binding affinity of an 83P5G4 monoclonal antibody is determined using standard technologies. Affinity measurements quantify the strength of antibody to epitope binding and can be used to help define which 83P5G4 monoclonal antibodies are preferred for diagnostic or therapeutic use. The BIAcore system (Uppsala, Sweden) is a preferred method for determining binding affinity. The BIAcore system uses surface plasmon resonance (SPR, Welford K. 1991, Opt. Quant. Elect. 23:1; Morton and Myszka, 1998, Methods in Enzymology 295:268) to monitor biomolecular interactions in real time. BIAcore analysis conveniently generates association rate constants, dissociation rate constants, equilibrium dissociation constants, and affinity constants.

### Example 10: In Vivo Assay for 83P5G4 Tumor Growth Promotion

The effect of the 83P5G4 protein on tumor cell growth can be evaluated in vivo by gene overexpression in tumor-bearing mice. For example, SCID mice can be injected SQ on each flank with 1×10⁶ of either PC3, TSUPR1, or DU145 cells containing tkNeo empty vector or 83P5G4. At least two strategies may be used: (1) Constitutive 83P5G4 expression under regulation of a promoter such as a constitutive promoter obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), or from heterologous mammalian promoters, e.g., the actin promoter or an immunoglobulin promoter, provided such promoters are compatible with the host cell systems. (2) Regulated expression under control of an inducible vector system, such as ecdysone, tet, etc., can be used provided such promoters are compatible with the host cell systems. Tumor volume is then monitored at the appearance of palpable tumors and is followed over time to determine if 83P5G4-expressing cells grow at a faster rate and whether tumors produced by 83P5G4-expressing cells demonstrate characteristics of altered aggressiveness (e.g. enhanced metastasis, vascularization, reduced responsiveness to chemotherapeutic drugs). Additionally, mice can be implanted with 1×10⁵ of the same cells orthotopically to determine if 83P5G4 has an effect on local growth in the prostate or on the ability of the cells to metastasize, specifically to lungs, lymph nodes, and bone marrow.

The assay is also useful to determine the 83P5G4 inhibitory effect of candidate therapeutic compositions, such as for example, 83P5G4 intrabodies, 83P5G4 antisense molecules and ribozymes.

### Example 11: Western Analysis of 83P5G4 Expression in Subcellular Fractions

The cellular location of 83P5G4 can be assessed using subcellular fractionation techniques widely used in cellular biology (Storrie B, et al. Methods Enzymol. 1990;182:203-25). Prostate or other cell lines can be separated into nuclear, cytosolic and membrane fractions. The expression of 83P5G4 in the different fractions can be tested using Western blotting techniques.

Alternatively, to determine the subcellular localization of 83P5G4, 293T cells can be transfected with an expression vector encoding HIS-tagged 83P5G4 (PCDNA 3.1 MYC/HIS, Invitrogen). The transfected cells can be harvested and subjected to a differential subcellular fractionation protocol as previously described (Pemberton, P. A. et al, 1997, J of Histochemistry and Cytochemistry, 45:1697-1706.) This protocol separates the cell into fractions enriched for nuclei, heavy membranes (lysosomes, peroxisomes, and mitochondria), light membranes (plasma membrane and endoplasmic reticulum), and soluble proteins.

### Example 12: Functional Evaluation of 83P5G4.

The 83P5G4 protein carries five WD-40 motifs, two CTF/NFI motifs and a leucine zipper. WD-40 is a motif first identified in beta subunits of trimeric G proteins that participate in G protein function. G-proteins function in signal transduction by physically interacting with a variety of proteins, including proteins carrying TPR motifs (van der Voom L, Ploegh H L. FEBS Let. 1992; 307:131). Several WD-40 containing proteins have been associated with cancer, including SG2NA, a gene expressed in S and G2 phases of cell growth, and MAWD, a gene overexpressed in breast cancer (Muro Y et al, Biochem. Biophys. Res. Commun. 1995, 207:1029; Matsuda S et al. Cancer Res. 2000, 60:13). These genes play a role in the growth and transformation of cells, and are therefore critical for the process of tumor formation. When 83P5G4 regulates the growth and transformation of cells, 83P5G4 is used as a target for diagnostic, preventative and therapeutic purposes.

Leucine zipper domains are involved in protein dimerization and determine sequence specific DNA binding (Luscher B, Larsson L G. Oncogene 1999;18:2955). CTF/NFI proteins represent a family of nuclear proteins that bind to CCAAT box and regulate both DNA replication and the transcription of mammalian genes (Gronostajski R M, Gene 2000; 249:3). Several leucine zipper-containing proteins have been associated with tumor progression, including MTA1, a gene expressed in most tumor cell lines that plays a role in tumor growth. Most proteins carrying the motifs mentioned above are understood to regulate critical processes such as cell division, gene transcription, transmembrane signaling, and vesicular trafficking (Neer E. et al. 1994, Nature 371, 297-300; Eugster A, Frigerio G, Dale M, Duden R. EMBO J. 2000;19: 3905; Solban N. et al. J Biol Chem. 2000; 275:32234). 83P5G4 carries out similarly essential functions in cancer cells. When 83P5G4 regulates critical processes such as cell division, gene transcription, transmembrane signaling, and vesicular trafficking, 83P5G4 is used as a target for diagnostic, preventative and therapeutic purposes.

Due to its similarity to Drosophila heat shock protein (HSP) L2dte (Gene 1996, 171-163), 83P5G4 may function as a heat shock protein, associate with various cellular proteins, and regulate their localization. When 83P5G4 functions as a heat shock protein, it can be used as a target for therapeutic intervention in accordance with techniques known in the art and in view of this disclosure.

### Example 13: Involvement of 83P5G4 in Cell Growth and Transformation.

83P5G4 contributes to the growth of prostate cancer and other tumor cells. Two sets of experiments evaluate this function. In the first set of experiments, PC3 cells engineered to stably express 83P5G4 are evaluated for cell growth potential. In a second set of experiments, primary prostate epithelial cells (PrEC) are engineered to express 83P5G4, and are evaluated for proliferation using a well-documented calorimetric assay (Johnson D E, Ochieng J, Evans S L. Anticancer Drugs. 1996, 7:288). In both cases, 83P5G4-expressing cells are compared to cells lacking 83P5G4 under resting and activating conditions. When 83P5G4 contributes to the growth of prostate cancer and other tumor cells, 83P5G4 is used as a target for diagnostic, preventative and therapeutic purposes.

In parallel to proliferation assays, the role of 83P5G4 in transformation can be evaluated. Primary PrEC cells and NIH3T3 cells engineered to express 83P5G4 are compared to parental 83P5G4-negative for their ability to form colonies in soft agar (Song Z. et al. Cancer Res. 2000;60:6730). This experiment measures the transforming capability of 83P5G4 and provides key information regarding the role of 83P5G4 in tumorigenesis. The function of 83P5G4 can be evaluated using anti-sense RNA technology coupled to the various functional assays described above, e.g. growth transformation. Anti-sense RNA oligonucleotides can be introduced into 83P5G4-expressing cells, thereby preventing the expression of 83P5G4. Control and anti-sense containing cells can be analyzed for proliferation, transformation and other tumor progression pathways listed below. The local as well as systemic effect of the loss of 83P5G4 expression can be evaluated. When 83P5G4 contributes to cell transformation, 83P5G4 is used as a target for diagnostic, preventative and therapeutic purposes.

### Example 14: Regulation of Cell Cycle and Apoptosis by 83P5G4.

Several proteins with WD-40 motifs regulate cell division and cell death. Similarly, 83P5G4 plays a role in cell cycle and apoptosis. For example, PC3-83P5G4 cells are compared to 83P5G4-negative PC3 for differences in cell cycle regulation using a well-established BrdU assay (Abdel-Malek Z A. J Cell Physiol. 1988, 136:247). In short, cells grown under both optimal (full serum) and limiting (low serum) conditions are labeled with BrdU for 1 hour and stained with anti-BrdU Ab and propidium iodide. Cells are analyzed for entry into the G1, S, and G2M phases of the cell cycle.

The 83P5G4 protein can prevent or enhance programmed cell death. The effect stress and chemotherapeutics on apoptosis is evaluated in 83P5G4-negative PC3 and PC3-83P5G4 cells. PC3 cells treated with various chemotherapeutic agents and protein synthesis inhibitors are stained with annexin V-FITC. Cell death is measured by FACS analysis. When 83P5G4 contributes to cell division and/or apoptosis, 83P5G4 is used as a target for diagnostic, preventative and therapeutic purposes.

### Example 15: Regulation of Transcription by 83P5G4.

The 83P5G4 protein contains several protein-protein interaction domains, as well as protein-DNA interaction domains. This, coupled to the presence of a leucine zipper motif within 83P5G4, indicates that 83P5G4 plays a role in transcriptional regulation of eukaryotic genes. Moreover, two nested nuclear localization sequences, each relatively non-specific, were identified by a PSORT prediction. In accordance with these findings, 83P5G4 protein regulates tumor growth by regulating gene expression. Regulation of gene expression can be evaluated by studying gene expression in cells expressing or lacking 83P5G4. For this purpose, two types of experiments can be performed. In the first set of experiments, RNA from parental and 83P5G4-expressing NIH3T3 and PC3 cells are extracted and hybridized to commercially available gene arrays (Clontech). Resting cells as well as cells treated with FBS or androgen are compared. Differentially expressed genes are identified in accordance with procedures known in the art. The differentially expressed genes can then be mapped to biological pathways. In the second set of experiments, specific transcriptional pathway activation is evaluated using commercially available (Stratagene) luciferase reporter constructs including: NFkB-luc, SRE-luc, ELK1-luc, ARE-luc, p53-luc, and CRE-luc. When 83P5G4 plays a role in gene regulation, 83P5G4 is used as a target for diagnostic, preventative and therapeutic purposes.

Throughout this application, various publications are referenced (within parentheses for example). The disclosures of these publications are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE I
Tissues that can Express 83P5G4 When Malignant (see, e.g. FIGS. 4-9)

| | | | |
|---|---|---|---|
| Prostate | Cervical | Stomach | Lung |
| Bladder | Uterine | Colon | Testicular |
| Kidney | Ovarian | Rectal | Small Intestine |
| Brain | Breast | Leukocytic | |
| Bone | Pancreatic | Liver | |

TABLE IIA
AMINO ACID ABBREVIATIONS

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gin | glutamine |
| R | Arg | arginine |
| I | lie | isoleucine |
| M | Met | methionine |
| T | Tin | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

TABLE IIB
AMINO ACID SUBSTITUTION MATRIX
Adapted from the GCG Software 9.0 BLOSUM62 amino acid substitution matrix (block substitution
matrix). The higher the value, the more likely a substitution is found in related, natural proteins.

| A | C | D | E | F | G | H | I | K | L | M | N | P | Q | R | S | T | V | W | Y | |
|---|---|---|---|---|---|---|---|---|---|---|---|---|---|---|---|---|---|---|---|---|
| 4 | 0 | −2 | −1 | −2 | 0 | −2 | −1 | −1 | −1 | −1 | −2 | −1 | −1 | −1 | 1 | 0 | 0 | −3 | −2 | A |
| | 9 | −3 | −4 | −2 | −3 | −3 | −1 | −3 | −1 | −1 | −3 | −3 | −3 | −3 | −1 | −1 | −1 | −2 | −2 | C |
| | | 6 | 2 | −3 | −1 | −1 | −3 | −1 | −4 | −3 | 1 | −1 | 0 | −2 | 0 | −1 | −3 | −4 | −3 | D |
| | | | 5 | −3 | −2 | 0 | −3 | 1 | −3 | −2 | 0 | −1 | 2 | 0 | 0 | −1 | −2 | −3 | −2 | E |
| | | | | 6 | −3 | −1 | 0 | −3 | 0 | 0 | −3 | −4 | −3 | −3 | −2 | −2 | −1 | 1 | 3 | F |
| | | | | | 6 | −2 | −4 | −2 | −4 | −3 | 0 | −2 | −2 | −2 | 0 | −2 | −3 | −2 | −3 | G |
| | | | | | | 8 | −3 | −1 | −3 | −2 | 1 | −2 | 0 | 0 | −1 | −2 | −3 | −2 | 2 | H |
| | | | | | | | 4 | −3 | 2 | 1 | −3 | −3 | −3 | −3 | −2 | −1 | 3 | −3 | −1 | I |
| | | | | | | | | 5 | −2 | −1 | 0 | −1 | 1 | 2 | 0 | −1 | −2 | −3 | −2 | K |
| | | | | | | | | | 4 | 2 | −3 | −3 | −2 | −2 | −2 | −1 | 1 | −2 | −1 | L |
| | | | | | | | | | | 5 | −2 | −2 | 0 | −1 | −1 | −1 | 1 | −1 | −1 | M |
| | | | | | | | | | | | 6 | −2 | 0 | 0 | 1 | 0 | −3 | −4 | −2 | N |
| | | | | | | | | | | | | 7 | −1 | −2 | −1 | −1 | −2 | −4 | −3 | P |
| | | | | | | | | | | | | | 5 | 1 | 0 | −1 | −2 | −2 | −1 | Q |
| | | | | | | | | | | | | | | 5 | −1 | −1 | −3 | −3 | −2 | R |
| | | | | | | | | | | | | | | | 4 | 1 | −2 | −3 | −2 | S |
| | | | | | | | | | | | | | | | | 5 | 0 | −2 | −2 | T |
| | | | | | | | | | | | | | | | | | 4 | −3 | −1 | V |
| | | | | | | | | | | | | | | | | | | 11 | 2 | W |
| | | | | | | | | | | | | | | | | | | | 7 | Y |

TABLE IIIA
HLA CLASS I SUPERMOTIFS

| SUPERMOTIF | POSITION 2 | C-TERMINUS |
|---|---|---|
| A2 | L,I,V,M,A,T,Q | L,.I,V,M,A,T |
| A3 | A,V,I,L,M,S,T | R,K |
| B7 | P | A,L,I,M,V,F,W,Y |
| B44 | D,E | F,W,Y,L,I,M,V,A |
| A1 | T,S,L,I,V,M | F,W,Y |
| A24 | F,W,Y,L,V,I,M,T | F,I,Y,W,L,M |
| B27 | R,H,K | A,L,I,V,M,Y,F,W |
| B58 | A,S,T | F,W,Y,L,I,V |
| B62 | L,V,M,P,I,Q | F,W,Y,M,I,V |

TABLE IIIB
HLA CLASS II SUPERMOTIF

| 1 | 6 | 9 |
|---|---|---|
| W.F.Y.V.I.L | A,V,I,L,P,C,S,T | A,V,I,L,C,S,T,M,Y |

TABLE IV
Scoring Results 83P5G4 HLA peptides A1 9-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 80 | NTESQSFRK | 225.000 |
| 2 | 618 | ISEPPSPIS | 27.000 |
| 3 | 544 | CSESRNRVK | 27.000 |
| 4 | 290 | CTDDNIYMF | 25.000 |
| 5 | 700 | TITPSSMRK | 10.000 |
| 6 | 540 | QAEACSESR | 9.000 |
| 7 | 515 | ITPPASETK | 5.000 |
| 8 | 337 | SDEAAYIWK | 4.500 |
| 9 | 580 | QVENLHLDL | 4.500 |
| 10 | 129 | AGELIGTCK | 4.500 |
| 11 | 379 | CSDDNTLKI | 3.750 |
| 12 | 266 | GSSTRKLGY | 3.750 |
| 13 | 427 | QSTPAKAPR | 3.000 |
| 14 | 191 | TSDKQTPSK | 3.000 |
| 15 | 585 | HLDLCCLAG | 2.500 |
| 16 | 554 | RLDSSCLES | 2.500 |
| 17 | 71 | NEEGFVRLY | 2.250 |
| 18 | 602 | SLGPTKSSK | 2.000 |
| 19 | 671 | KAENPSPRS | 1.800 |
| 20 | 336 | SSDEAAYIW | 1.500 |
| 21 | 646 | GSEMVGKEN | 1.350 |
| 22 | 253 | RQEPIASKS | 1.350 |
| 23 | 304 | KTSPVAIFN | 1.250 |
| 24 | 643 | CGEGSEMVG | 1.125 |
| 25 | 262 | FLYPGSSTR | 1.000 |
| 26 | 599 | SKDSLGPTK | 1.000 |
| 27 | 145 | SVAFSKFEK | 1.000 |
| 28 | 559 | CLESVKQKC | 0.900 |
| 29 | 62 | NMEHVLAVA | 0.900 |
| 30 | 610 | KIEGAGTSI | 0.900 |
| 31 | 519 | ASETKIMSP | 0.675 |
| 32 | 326 | SPDDQFLVS | 0.625 |
| 33 | 194 | KQTPSKPKK | 0.600 |
| 34 | 424 | TSSQSTPAK | 0.600 |
| 35 | 462 | SNTPTFSIK | 0.500 |
| 36 | 404 | STVGWASQK | 0.500 |
| 37 | 212 | SVDFQQSVT | 0.500 |
| 38 | 693 | KTLPSPVTI | 0.500 |
| 39 | 235 | AVDGIIKVW | 0.500 |
| 40 | 195 | QTPSKPKKK | 0.500 |
| 41 | 576 | ELDGQVENL | 0.500 |
| 42 | 167 | VWDTRCNKK | 0.500 |
| 43 | 69 | VANEEGFVR | 0.500 |
| 44 | 278 | ILDSTGSTL | 0.500 |
| 45 | 367 | CWCPSDFTK | 0.500 |
| 46 | 470 | KTSPAKARS | 0.500 |
| 47 | 45 | ETGVPVPPF | 0.500 |
| 48 | 70 | ANEEGFVRL | 0.450 |
| 49 | 370 | PSDFTKIAT | 0.375 |
| 50 | 224 | FQDENTLVS | 0.375 |

TABLE V
Scoring Results 83P5G4 HLA peptides A1 10-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 336 | SSDEAAYIWK | 75.000 |
| 2 | 80 | NTESQSFRKK | 45.0000 |
| 3 | 544 | CSESRNRVKR | 27.000 |
| 4 | 393 | GLEEKIPGGDK | 18.000 |
| 5 | 519 | ASETKIMSPR | 13.500 |
| 6 | 253 | RQEPIASKSF | 13.500 |
| 7 | 70 | ANEEGFVRLY | 11.250 |
| 8 | 643 | CGEGSEMVGK | 9.000 |
| 9 | 618 | ISEPPSPISP | 6.750 |
| 10 | 629 | ASESCGTLPL | 6.750 |
| 11 | 290 | CTDDNIYMFN | 6.250 |
| 12 | 278 | ILDSTGSTLF | 5.000 |
| 13 | 574 | VTELDGQVEN | 4.500 |
| 14 | 699 | VTITPSSMRK | 2.500 |
| 15 | 262 | FLYPGSSTRK | 2.000 |
| 16 | 333 | VSGSSDEAAY | 1.500 |
| 17 | 144 | KSVAFSKFEK | 1.500 |
| 18 | 646 | GSEMVGKENS | 1.350 |
| 19 | 304 | KTSPVAIFNG | 1.250 |
| 20 | 380 | SDDNTLKIWR | 1.250 |
| 21 | 656 | SPENKNWLLA | 1.125 |
| 22 | 488 | SVSPKPPSSF | 1.000 |
| 23 | 591 | LAGNQEDLSK | 1.000 |
| 24 | 585 | HLDLCCLAGN | 1.000 |
| 25 | 423 | VTSSQSTPAK | 1.000 |
| 26 | 232 | SAGAVDGIIK | 1.000 |
| 27 | 190 | NTSDKQTPSK | 1.000 |
| 28 | 212 | SVDFQQSVTV | 1.000 |
| 29 | 559 | CLESVKQKCV | 0.900 |
| 30 | 580 | QVENLHLDLC | 0.900 |
| 31 | 610 | KIEGAGTSIS | 0.900 |
| 32 | 651 | GKENSSPENK | 0.900 |
| 33 | 62 | NMEHVLAVAN | 0.900 |
| 34 | 540 | QAEACSESRN | 0.900 |
| 35 | 34 | CSGNDEHTSY | 0.750 |
| 36 | 379 | CSDDNTLKIW | 0.750 |
| 37 | 255 | EPIASKSFLY | 0.625 |
| 38 | 287 | FANCTDDNIY | 0.500 |
| 39 | 68 | AVANEEGFVR | 0.500 |
| 40 | 404 | STVGWASQKK | 0.500 |
| 41 | 576 | ELDGQVENLH | 0.500 |
| 42 | 458 | LPLPSNTPTF | 0.500 |
| 43 | 554 | RLDSSCLESV | 0.500 |
| 44 | 208 | GLAPSVDFQQ | 0.500 |
| 45 | 114 | VTAAGDQTAK | 0.500 |
| 46 | 324 | SLSPDDQFLV | 0.500 |
| 47 | 428 | STPAKAPRVK | 0.500 |
| 48 | 377 | ATCSDDNTLK | 0.500 |
| 49 | 601 | DSLGPTKSSK | 0.300 |
| 50 | 557 | SSCLESVKQK | 0.300 |

TABLE VI
Scoring Results 83P5G4 HLA peptides A2 9-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 355 | VLLGHSQEV | 437.482 |
| 2 | 222 | VLFQDENTL | 134.369 |
| 3 | 324 | SLSPDDQFL | 117.493 |
| 4 | 106 | WVPGELKLV | 64.388 |
| 5 | 498 | KMSIRNWVT | 57.924 |
| 6 | 583 | NLHLDLCCL | 49.134 |
| 7 | 523 | KIMSPRKAL | 38.038 |
| 8 | 271 | KLGYSSLIL | 30.655 |
| 9 | 92 | KEWMAHWNA | 21.047 |
| 10 | 325 | LSPDDQFLV | 18.354 |
| 11 | 386 | KIWRLNRGL | 17.066 |
| 12 | 278 | ILDSTGSTL | 14.526 |
| 13 | 524 | IMSPRKALI | 12.809 |
| 14 | 99 | NAVFDLAWV | 12.220 |
| 15 | 590 | CLAGNQEDL | 10.468 |
| 16 | 234 | GAVDGIIKVR | 9.109 |
| 17 | 497 | FKMSINWV | 9.043 |
| 18 | 119 | DQTAKFWDV | 7.537 |
| 19 | 694 | TLPSPVTIT | 7.027 |
| 20 | 21 | SQYPLQSLL | 6.931 |
| 21 | 617 | SISEPPSPI | 5.881 |
| 22 | 6 | ALRQPQLGV | 5.286 |
| 23 | 68 | AVANEEGFV | 4.351 |
| 24 | 215 | FQQSVTVVL | 4.085 |
| 25 | 277 | LILDSTGST | 3.435 |
| 26 | 573 | CVTELDGQV | 3.244 |
| 27 | 655 | SSPENKNWL | 3.145 |
| 28 | 356 | LLGHSQEVT | 2.545 |
| 29 | 164 | NIMVWDTRC | 2.527 |
| 30 | 47 | GVPVPPFGC | 2.521 |
| 31 | 560 | LESVKQKCV | 2.299 |
| 32 | 635 | TLPLPLRPC | 2.285 |
| 33 | 221 | VVLFQDENT | 2.010 |
| 34 | 402 | KLSTVGWAS | 1.956 |
| 35 | 693 | KTLPSPVTI | 1.876 |
| 36 | 543 | ACSESRNRV | 1.861 |
| 37 | 366 | VCWCPSDFT | 1.850 |
| 38 | 338 | DEAAYIWKV | 1.750 |
| 39 | 96 | AHWNAVFDL | 1.643 |
| 40 | 300 | MTGLKTSPV | 1.642 |
| 41 | 566 | KCVKSCNCV | 1.589 |
| 42 | 313 | GHQNSTFYV | 1.541 |
| 43 | 335 | GSSDEAAYI | 1.536 |
| 44 | 361 | QEVTSVCWC | 1.222 |
| 45 | 180 | RQVNQISGA | 1.159 |
| 46 | 17 | NGWSSQYPL | 1.157 |
| 47 | 569 | KSCNCVTEL | 1.123 |
| 48 | 317 | STFYVKSSL | 1.098 |
| 49 | 347 | STPWQPPTV | 0.966 |
| 50 | 428 | STPAKAPRV | 0.966 |

TABLE VII
Scoring Results 83P5G4 HLA peptides A2 10-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 222 | VLFQDENTLV | 437.482 |
| 2 | 241 | KVWDLRKNYT | 427.143 |
| 3 | 324 | SLSPDDQFLV | 403.402 |
| 4 | 296 | YMFNMTGLKT | 91.602 |
| 5 | 277 | LILDSTGSTL | 75.751 |
| 6 | 92 | KEWMAHWNAV | 66.788 |
| 7 | 299 | NMTGLKTSPV | 50.232 |
| 8 | 554 | RLDSSCLESV | 31.354 |
| 9 | 98 | WNAVFDLAWV | 26.419 |
| 10 | 146 | VAFSKFEKAV | 23.089 |
| 11 | 354 | TVLLGHSQEV | 22.517 |
| 12 | 294 | NIYMFNMTGL | 21.619 |
| 13 | 523 | KIMSPRKALI | 18.577 |
| 14 | 312 | NGHQNSTFYV | 14.483 |
| 15 | 112 | KLVTAAGDQT | 12.780 |
| 16 | 221 | VVLFQDENTL | 11.757 |
| 17 | 331 | FLVSGSSDEA | 11.198 |
| 18 | 414 | KESRPGLVTV | 10.887 |
| 19 | 602 | SLGPTKSSKI | 10.433 |
| 20 | 355 | VLLGHSQEVT | 9.417 |
| 21 | 579 | GQVENLHLDL | 8.880 |
| 22 | 224 | FQDENTLVSA | 8.740 |
| 23 | 575 | TELDGQVENL | 7.102 |
| 24 | 95 | MAHWNAVFDL | 6.729 |
| 25 | 583 | NLHLDLCCLA | 4.968 |
| 26 | 559 | CLESVKQKCV | 4.451 |
| 27 | 655 | SSPENKNWLL | 4.288 |
| 28 | 104 | LAWVPGELKL | 4.186 |
| 29 | 69 | VANEEGFVRL | 3.929 |
| 30 | 309 | AIFNGHQNST | 3.791 |
| 31 | 397 | KPGGDKLSTV | 3.655 |
| 32 | 137 | KGHQCSLKSV | 3.655 |
| 33 | 5 | SALRQPQLGV | 3.574 |
| 34 | 323 | SSLSPDDQFL | 2.838 |
| 35 | 212 | SVDFQQSVTV | 2.434 |
| 36 | 21 | SQYPLQSLLT | 2.418 |
| 37 | 57 | FSSAPNMEHV | 2.354 |
| 38 | 254 | QEPIASKSFL | 2.285 |
| 39 | 34 | KVSTPWQPPT | 2.282 |
| 40 | 149 | SKFEKAVFCT | 2.095 |
| 41 | 357 | LGHSQEVTSV | 1.775 |
| 42 | 525 | MSPRKALIPV | 1.775 |
| 43 | 375 | KIATCSDDNT | 1.757 |
| 44 | 365 | SVCWCPSDFT | 1.757 |
| 45 | 230 | LVSAGAVDGI | 1.749 |
| 46 | 210 | APSVDFQQSV | 1.725 |
| 47 | 32 | YQCSGNDEHT | 1.703 |
| 48 | 663 | LLAMAAKRKA | 1.689 |
| 49 | 19 | WSSQYPLQSL | 1.475 |
| 50 | 41 | TSYGETGVPV | 1.453 |

TABLE VIII
Scoring Results 83P5G4 HLA peptides A3 9-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 142 | SLKSVAFSK | 90.000 |
| 2 | 708 | KICTYFHRK | 54.000 |
| 3 | 602 | SLGPTKSSK | 30.000 |
| 4 | 389 | RLNRGLEEK | 30.000 |
| 5 | 296 | YMFNMTGLK | 30.000 |
| 6 | 262 | FLYPGSSTR | 30.000 |
| 7 | 384 | TLKIWRLNR | 24.000 |
| 8 | 239 | IIKVWDLRK | 12.966 |
| 9 | 166 | LLAMAARK | 10.000 |
| 10 | 404 | MVWDTRCNK | 6.750 |
| 11 | 94 | STVGWASQK | 6.000 |
| 12 | 700 | WMAHWNAVF | 6.000 |
| 13 | 700 | TITPSSMRK | 6.000 |
| 14 | 145 | SVAFSKFEK | 6.000 |
| 15 | 662 | WLLAMAAKR | 6.000 |
| 16 | 314 | HQNSTFYVK | 5.400 |
| 17 | 529 | KALIPVSQK | 4.050 |
| 18 | 271 | KLGYSSLIL | 3.600 |
| 19 | 705 | SMRKICTYF | 3.000 |
| 20 | 120 | QTAKFWDVK | 3.000 |
| 21 | 222 | VLFQDENTL | 3.000 |
| 22 | 80 | NTESQSFRK | 3.000 |
| 23 | 241 | KVWDLRKNY | 3.000 |
| 24 | 194 | KQTPSKPKK | 2.700 |
| 25 | 238 | GIIKWDLR | 2.700 |
| 26 | 405 | TVGWASQKK | 2.000 |
| 27 | 302 | GLKTSPVAI | 1.800 |
| 28 | 104 | LAWVPGELK | 1.500 |
| 29 | 515 | ITPPASETK | 1.500 |
| 30 | 484 | GSVSSVSPK | 1.350 |
| 31 | 244 | DLRKNYTAY | 1.200 |
| 32 | 498 | KMSIRNWVT | 0.900 |
| 33 | 324 | SLSPDDQFL | 0.900 |
| 34 | 583 | NLHLDLCCL | 0.900 |
| 35 | 524 | IMSPRKALI | 0.900 |
| 36 | 590 | CLAGNQEDL | 0.900 |
| 37 | 576 | LDGQVENL | 0.810 |
| 38 | 558 | SCLESVKQK | 0.675 |
| 39 | 99 | KPKKKQNSK | 0.600 |
| 40 | 278 | ILDSTGSTL | 0.600 |
| 41 | 6 | ALRQPQLGV | 0.600 |
| 42 | 24 | PLQSLLTGY | 0.600 |
| 43 | 402 | KLSTVGWAS | 0.540 |
| 44 | 195 | QTPSKPKKK | 0.500 |
| 45 | 84 | QSFRKKCFK | 0.500 |
| 46 | 490 | SPKPPSSFK | 0.450 |
| 47 | 699 | VTITPSSMR | 0.450 |
| 48 | 355 | VLLGHSQEV | 0.450 |
| 49 | 694 | TLPSPVTIT | 0.450 |
| 50 | 62 | NMEHVLAVA | 0.450 |

TABLE IX
Scoring Results 83P5G4 HLA peptides A3 10-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 262 | FLYPGSSTRK | 150.000 |
| 2 | 238 | GIIKVWDLRK | 54.000 |
| 3 | 393 | GLEEKPGGDK | 40.500 |
| 4 | 165 | IMVWDTRCNK | 30.000 |
| 5 | 302 | GLKTSPVAIF | 27.000 |
| 6 | 662 | WLLAMAAKRK | 15.000 |
| 7 | 498 | KMSIRNWVTR | 12.000 |
| 8 | 14 | VLRNGWSSQY | 12.000 |
| 9 | 166 | MVWDTRCNKK | 10.000 |
| 10 | 77 | RLYNTESQSF | 10.000 |
| 11 | 103 | DLAWVPGELK | 9.000 |
| 12 | 142 | SLKSVAFSKF | 6.000 |
| 13 | 244 | DLRKNYTAYR | 3.600 |
| 14 | 366 | VCWCPSDFTK | 3.000 |
| 15 | 699 | VTITPSSMRK | 3.000 |
| 16 | 404 | STVGWASQKK | 2.250 |
| 17 | 66 | VLAVANEEGF | 2.000 |
| 18 | 278 | ILDSTGSTLF | 2.000 |
| 19 | 383 | NTLKIWRLNR | 1.800 |
| 20 | 144 | KSVAFSKFEK | 1.350 |
| 21 | 194 | KQTPSKPKKK | 1.322 |
| 22 | 128 | KAGELIGTCK | 1.350 |
| 23 | 68 | AVANEEGFVR | 1.200 |
| 24 | 296 | YMFNMTGLKT | 1.000 |
| 25 | 190 | NTSDKQTPSK | 1.000 |
| 26 | 377 | ATCSDDNTLK | 1.000 |
| 27 | 423 | VTSSQSTPAK | 1.000 |
| 28 | 405 | TVGWASQKKK | 1.000 |
| 29 | 222 | VLFQDENTLV | 1.000 |
| 30 | 114 | VTAAGDQTAK | 0.900 |
| 31 | 660 | KNWLLAMAAK | 0.900 |
| 32 | 324 | SLSPDDQFLV | 0.900 |
| 33 | 602 | SLGPTKSSKI | 0.900 |
| 34 | 141 | CSLKSVAFSK | 0.675 |
| 35 | 705 | SMRKICTYFH | 0.600 |
| 36 | 83 | SQSFRKKCFK | 0.600 |
| 37 | 119 | DQTAKFWDVK | 0.540 |
| 38 | 313 | GHQNSTFYVK | 0.540 |
| 39 | 112 | KLVTAAGDQT | 0.450 |
| 40 | 488 | SVSPKPPSSF | 0.450 |
| 41 | 294 | NIYMFNMTGL | 0.450 |
| 42 | 208 | GLAPSVDFQQ | 0.405 |
| 43 | 591 | LAGNQEDLSK | 0.400 |
| 44 | 232 | SAGAVDGIIK | 0.400 |
| 45 | 299 | NMTGLKTSPV | 0.300 |
| 46 | 554 | RLDSSCLESV | 0.300 |
| 47 | 336 | SSDEAAYIWK | 0.300 |
| 48 | 331 | FLVSGSSDEA | 0.300 |
| 49 | 135 | TCKGHQCSLK | 0.300 |
| 50 | 468 | SIKTSPAKAR | 0.300 |

TABLE X
Scoring Results 83P5G4 HLA peptides A11 9-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 145 | SVAFSKFEK | 6.000 |
| 2 | 166 | MVWDTRCNK | 4.000 |
| 3 | 80 | NTESQSFRK | 3.000 |
| 4 | 405 | TVGWASQKK | 2.000 |
| 5 | 194 | KQTPSKPKK | 1.800 |
| 6 | 404 | STVGWASQK | 1.500 |
| 7 | 314 | HQNSTFYVK | 1.200 |
| 8 | 708 | KICTYFHRK | 1.200 |
| 9 | 389 | RLNRGLEEK | 1.200 |
| 10 | 142 | SLKSVAFSK | 1.200 |
| 11 | 515 | ITPPASETK | 1.000 |
| 12 | 120 | QTAKFWDVK | 1.000 |
| 13 | 529 | KALIPVSQK | 0.900 |
| 14 | 700 | TITPSSMRK | 0.800 |
| 15 | 239 | IIKVWDLRK | 0.800 |
| 16 | 296 | YMFNMTGLK | 0.800 |
| 17 | 199 | KPKKKQNSK | 0.600 |
| 18 | 195 | QTPSKPKKK | 0.500 |
| 19 | 263 | LYPGSSTRK | 0.400 |
| 20 | 104 | LAWVPGELK | 0.400 |
| 21 | 602 | SLGPTKSSK | 0.400 |
| 22 | 8 | RQPQLGVLR | 0.360 |
| 23 | 238 | GIIKVWDLR | 0.360 |
| 24 | 521 | ETKIMSPRK | 0.300 |
| 25 | 699 | VTITPSSMR | 0.300 |
| 26 | 115 | TAAGDQTAK | 0.200 |
| 27 | 490 | SPKPPSSFK | 0.200 |
| 28 | 663 | LLAMAAKRK | 0.200 |
| 29 | 378 | TCSDDNTLK | 0.200 |
| 30 | 153 | KAVFCTGGR | 0.180 |
| 31 | 644 | GEGSEMVGK | 0.180 |
| 32 | 652 | KENSSPENK | 0.180 |
| 33 | 262 | FLYPGSSTR | 0.160 |
| 34 | 384 | TLKIWRLNR | 0.160 |
| 35 | 558 | SCLESVKQK | 0.150 |
| 36 | 475 | KARSPINRR | 0.120 |
| 37 | 662 | WLLAMAAKR | 0.120 |
| 38 | 69 | VANEEGFVR | 0.120 |
| 39 | 484 | GSVSSVSPK | 0.090 |
| 40 | 367 | CWCPSDFTK | 0.060 |
| 41 | 241 | KVWDLRKNY | 0.060 |
| 42 | 394 | LEEKPGGDK | 0.060 |
| 43 | 707 | RKICTYFHR | 0.054 |
| 44 | 693 | KTLPSPVTI | 0.045 |
| 45 | 592 | AGNQEDLSK | 0.040 |
| 46 | 580 | QVENLHLDL | 0.040 |
| 47 | 462 | SNTPTFSIK | 0.040 |
| 48 | 84 | QSFRKKCFK | 0.040 |
| 49 | 337 | SDEAAYIWK | 0.040 |
| 50 | 233 | AGAVDGIIK | 0.040 |

TABLE XI
Scoring Results 83P5G4 HLA peptides A3 10-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 166 | MVWDTRCNKK | 4.000 |
| 2 | 238 | GIIKVWDLRK | 3.600 |
| 3 | 699 | VTITPSSMRK | 3.000 |
| 4 | 404 | STVGWASQKK | 1.500 |
| 5 | 393 | GLEEKPGGDK | 1.200 |
| 6 | 366 | VCWCPSDFTK | 1.200 |
| 7 | 68 | AVANEEGFVR | 1.200 |
| 8 | 423 | VTSSQSTPAK | 1.000 |
| 9 | 405 | TVGWASQKKK | 1.000 |
| 10 | 190 | NTSDKQTPSK | 1.000 |
| 11 | 114 | VTAAGDQTAK | 1.000 |
| 12 | 377 | ATCSDDNTLK | 1.000 |
| 13 | 194 | KQTPSKPKKK | 0.900 |
| 14 | 295 | IYMPNMTGLK | 0.800 |
| 15 | 262 | FLYPGSSTRK | 0.800 |
| 16 | 83 | SQSFRKKCFK | 0.600 |
| 17 | 165 | IMVWDTRCNK | 0.600 |
| 18 | 128 | KAGELIGTCK | 0.600 |
| 19 | 383 | NTLKIWRLNR | 0.600 |
| 20 | 232 | SAGAVDGIIK | 0.400 |
| 21 | 591 | LAGNQEDLSK | 0.400 |
| 22 | 251 | AYRQEPIASK | 0.400 |
| 23 | 171 | RCNKKDGFYR | 0.360 |
| 24 | 662 | WLLAMAAKRK | 0.300 |
| 25 | 144 | KSVAFSKFEK | 0.270 |
| 26 | 660 | KNWLLAMAAK | 0.240 |
| 27 | 498 | KMSIRNWVTR | 0.240 |
| 28 | 466 | TFSIKTSPAK | 0.200 |
| 29 | 135 | TCKGHQCSLK | 0.200 |
| 30 | 684 | TPNSRRQSGK | 0.200 |
| 31 | 119 | DQTAKFWDVK | 0.180 |
| 32 | 79 | YNTESQSFRK | 0.120 |
| 33 | 313 | GHQNSTFYVK | 0.120 |
| 34 | 103 | DLAWVPGELK | 0.120 |
| 35 | 539 | SQAEACSESR | 0.120 |
| 36 | 426 | SQSTPAKAPR | 0.120 |
| 37 | 428 | STPAKAPRVK | 0.100 |
| 38 | 80 | NTESQSFRKK | 0.100 |
| 39 | 707 | RKICTYFHRK | 0.090 |
| 40 | 141 | CSLKSVAFSK | 0.090 |
| 41 | 78 | LYNTESQSFR | 0.080 |
| 42 | 528 | RKALIPVSQK | 0.060 |
| 43 | 483 | RGSVSSVSPK | 0.060 |
| 44 | 651 | GKENSSPENK | 0.060 |
| 45 | 560 | LESVKQKCVK | 0.060 |
| 46 | 261 | SFLYPGSSTR | 0.060 |
| 47 | 520 | SETKIMSPRK | 0.060 |
| 48 | 579 | GQVENLHLDL | 0.054 |
| 49 | 336 | SSDEAAYIWK | 0.040 |
| 50 | 468 | SIKTSPAKAR | 0.040 |

TABLE XII
Scoring Results 83P5G4 HLA peptides A24 9-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 295 | IYMFNMTGL | 300.000 |
| 2 | 78 | LYNTESQSF | 180.000 |
| 3 | 523 | KIMSPRKAL | 12.000 |
| 4 | 386 | KIWRLNRGL | 9.600 |
| 5 | 569 | KSCNCVTEL | 8.800 |
| 6 | 655 | SSPENKNWL | 8.640 |
| 7 | 70 | ANEEGFVRL | 8.640 |
| 8 | 178 | FYRQVNQIS | 8.400 |
| 9 | 215 | FQQSVTVVL | 8.400 |
| 10 | 271 | KLGYSSLIL | 8.000 |
| 11 | 105 | AWVPGELKL | 7.920 |
| 12 | 722 | CGPEHSTEL | 7.920 |
| 13 | 22 | QYPLQSLLT | 7.500 |
| 14 | 580 | QVENLHLDL | 7.200 |
| 15 | 20 | SSQYPLQSL | 7.200 |
| 16 | 103 | DLAWVPGEL | 6.160 |
| 17 | 255 | EPIASKSFL | 6.000 |
| 18 | 578 | DGQVENLHL | 6.000 |
| 19 | 656 | SPENKNWLL | 6.000 |
| 20 | 42 | SYGETGVPV | 6.000 |
| 21 | 450 | CAPSCAGDL | 6.000 |
| 22 | 177 | GFYRQVNQI | 6.000 |
| 23 | 237 | DGIIKVWDL | 6.000 |
| 24 | 207 | KGLAPSVDF | 6.000 |
| 25 | 273 | GYSSLILDS | 6.000 |
| 26 | 59 | SAPNMEHVL | 6.000 |
| 27 | 21 | SQYPLQSLL | 5.760 |
| 28 | 324 | SLSPDDQFL | 5.760 |
| 29 | 317 | STFYVKSSL | 5.600 |
| 30 | 251 | AYRQEPIAS | 5.000 |
| 31 | 628 | YASESCGTL | 4.800 |
| 32 | 632 | SCGTLPLPL | 4.800 |
| 33 | 222 | VLFQDENTL | 4.800 |
| 34 | 377 | ATCSDDNTL | 4.800 |
| 35 | 264 | YPGSSTRKL | 4.400 |
| 36 | 124 | FWDVKAGEL | 4.400 |
| 37 | 583 | NLHLDLCCL | 4.000 |
| 38 | 576 | ELDGQVENL | 4.000 |
| 39 | 135 | TCKGHQCSL | 4.000 |
| 40 | 348 | TPWQPPTVL | 4.000 |
| 41 | 590 | CLAGNQEDL | 4.000 |
| 42 | 382 | DNTLKIWRL | 4.000 |
| 43 | 278 | ILDSTGSTL | 4.000 |
| 44 | 4 | NSALRQPQL | 4.000 |
| 45 | 17 | NGWSSQYPL | 4.000 |
| 46 | 693 | KTLPSPVTI | 3.600 |
| 47 | 610 | KIEGAGTSI | 3.000 |
| 48 | 507 | RTPSSSPPI | 3.000 |
| 49 | 323 | SSLSPDDQF | 3.000 |
| 50 | 489 | VSPKPPSSF | 3.000 |

TABLE XIII
Scoring Results 83P5G4 HLA peptides A3 9-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 263 | LYPGSSTRKL | 330.000 |
| 2 | 123 | KFWDVKAGEL | 52.800 |
| 3 | 248 | NYTAYRQEPI | 50.000 |
| 4 | 214 | DFQQSVTVVL | 42.000 |
| 5 | 627 | PYASESCGTL | 20.000 |
| 6 | 310 | IFNGHQNSTF | 15.000 |
| 7 | 341 | AYIWKVSTPW | 10.500 |
| 8 | 147 | AFSKFEKAVF | 10.000 |
| 9 | 712 | YFHRKSQEDF | 10.000 |
| 10 | 69 | VANEEGFVRL | 8.640 |
| 11 | 579 | GQVENLHLDL | 8.640 |
| 12 | 16 | RNGWSSQYPL | 8.000 |
| 13 | 277 | LILDSTGSTL | 7.200 |
| 14 | 323 | SSLSPDDQFL | 7.200 |
| 15 | 655 | SSPENKNWLL | 7.200 |
| 16 | 221 | VVLFQDENTL | 7.200 |
| 17 | 594 | NQEDLSKDSL | 7.200 |
| 18 | 253 | RQEPIASKSF | 7.200 |
| 19 | 20 | SSQYPLQSLL | 7.200 |
| 20 | 273 | GYSSLILDST | 7.000 |
| 21 | 629 | ASESCGTLPL | 6.000 |
| 22 | 582 | ENLHLDLCCL | 6.000 |
| 23 | 589 | CCLAGNQEDL | 6.000 |
| 24 | 347 | STPWQPPTVL | 6.000 |
| 25 | 654 | NSSPENKNWL | 5.760 |
| 26 | 546 | ESRNRVKRRL | 5.600 |
| 27 | 316 | NSTFYVKSSL | 5.600 |
| 28 | 721 | FCGPEHSTEL | 5.280 |
| 29 | 286 | LFANCTDDNI | 5.000 |
| 30 | 77 | RLYNTESQSF | 4.800 |
| 31 | 631 | ESCGTLPLPL | 4.800 |
| 32 | 376 | IATCSDDNTL | 4.800 |
| 33 | 449 | ACAPSCAGDL | 4.800 |
| 34 | 19 | WSSQYPLQSL | 4.800 |
| 35 | 58 | SSAPNMEHVL | 4.800 |
| 36 | 104 | LAWVPGELKL | 4.400 |
| 37 | 704 | SSMRKICTYF | 4.200 |
| 38 | 95 | MAHWNAVFDL | 4.000 |
| 39 | 348 | TPWQPPTVLL | 4.000 |
| 40 | 686 | NSRRQSGKTL | 4.000 |
| 41 | 268 | STRKLGYSSL | 4.000 |
| 42 | 451 | APSCAGDLPL | 4.000 |
| 43 | 134 | GTCKGHQCSL | 4.000 |
| 44 | 294 | NIYMFNMTGL | 4.000 |
| 45 | 6 | ALRQPQLGVL | 4.000 |
| 46 | 322 | KSSLSPDDQF | 4.000 |
| 47 | 3 | FNSALRQPQL | 4.000 |
| 48 | 48 | VPVPPFGCTF | 3.600 |
| 49 | 458 | LPLPSNTPTF | 3.600 |
| 50 | 492 | KPPSSFKMSI | 3.000 |

TABLE XIV
Scoring Results 83P5G4 HLA PEPTIDES B7 9-MERS

| | | | Score (Estimate of Half Time of |
|---|---|---|---|
| | Start | Subsequence Residue | Disassociation of a Molecule |
| Rank | Position | Listing | Containing This Subsequence) |
| 1 | 348 | TPWQPPTVL | 120.000 |
| 2 | 255 | EPIASKSFL | 80.000 |
| 3 | 264 | YPGSSTRIKL | 80.000 |
| 4 | 526 | SPRKALIPV | 40.000 |
| 5 | 676 | SPRSPSSQT | 30.000 |
| 6 | 523 | KIMSPRKAL | 27.000 |
| 7 | 656 | SPENKNWLL | 24.000 |
| 8 | 641 | RPCGEGSEM | 20.000 |
| 9 | 472 | SPAKARSPI | 12.000 |
| 10 | 59 | SAPNMEHVL | 12.000 |
| 11 | 628 | YASESCGTL | 12.000 |
| 12 | 433 | APRVKCNPS | 12.000 |
| 13 | 377 | ATCSDDNTL | 12.000 |
| 14 | 450 | CAPSCAGDL | 12.000 |
| 15 | 6 | ALRQPQLGV | 9.000 |
| 16 | 516 | TPPASETKI | 8.000 |
| 17 | 20 | SSQYPLQSL | 6.000 |
| 18 | 580 | QVENLHLDL | 6.000 |
| 19 | 478 | SPINRRGSV | 6.000 |
| 20 | 60 | APNMEHVLA | 6.000 |
| 21 | 655 | SSPENKNWL | 4.000 |
| 22 | 271 | KLGYSSLIL | 4.000 |
| 23 | 21 | SQYPLQSLL | 4.000 |
| 24 | 17 | NGWSSQYPL | 4.000 |
| 25 | 237 | DGIIKVWDL | 4.000 |
| 26 | 386 | KIWRLNRGL | 4.000 |
| 27 | 317 | STFYVKSSL | 4.000 |
| 28 | 324 | SLSPDDQFL | 4.000 |
| 29 | 632 | SCGTLPLPL | 4.000 |
| 30 | 583 | NLHLDLCCL | 4.000 |
| 31 | 590 | CLAGNQEDL | 4.000 |
| 32 | 4 | NSALRQPQL | 4.000 |
| 33 | 103 | DLAWVPGEL | 4.000 |
| 34 | 215 | FQQSVTVVL | 4.000 |
| 35 | 722 | CGPEHSTEL | 4.000 |
| 36 | 687 | SRRQSGKTL | 4.000 |
| 37 | 222 | VLFQDENTL | 4.000 |
| 38 | 135 | TCKGHQCSL | 4.000 |
| 39 | 382 | DNTLKIWRL | 4.000 |
| 40 | 578 | DGQVENLHL | 4.000 |
| 41 | 569 | KSCNCVTEL | 4.000 |
| 42 | 552 | KRRLDSSCL | 4.000 |
| 43 | 70 | ANEEGFVRL | 3.600 |
| 44 | 68 | AVANEEGFV | 3.000 |
| 45 | 48 | VPVPPFGCT | 3.000 |
| 46 | 702 | TPSSMRKIC | 3.000 |
| 47 | 107 | VPGELKLVT | 2.000 |
| 48 | 415 | ESRPGLVTV | 2.000 |
| 49 | 369 | CPSDFTKIA | 2.000 |
| 50 | 458 | LPLPSNTPT | 2.000 |

TABLE XV
Scoring Results 83P5G4 HLA PEPTIDES B7 10-MERS

| | | | Score (Estimate of Half Time of |
|---|---|---|---|
| | Start | Subsequence Residue | Disassociation of a Molecule |
| Rank | Position | Listing | Containing This Subsequence |
| 1 | 451 | APSCAGDLPL | 240.000 |
| 2 | 6 | ALRQPQLGVL | 120.000 |
| 3 | 348 | TPWQPPTVLL | 120.000 |
| 4 | 268 | STRKLGYSSL | 40.000 |
| 5 | 546 | ESRNRVKRRL | 40.000 |
| 6 | 686 | NSRRQSGKTL | 40.000 |
| 7 | 221 | VVLFQDENTL | 20.000 |
| 8 | 490 | SPKPPSSFKM | 20.000 |
| 9 | 697 | SPVTITPSSM | 20.000 |
| 10 | 516 | TPPASETKIM | 20.000 |
| 11 | 60 | APNMEHVLAV | 12.000 |
| 12 | 376 | IATCSDDNTL | 12.000 |
| 13 | 460 | LPSNTPTFSI | 12.000 |
| 14 | 69 | VANEEGFVRL | 12.000 |
| 15 | 95 | MAHWNAVFDL | 12.000 |
| 16 | 104 | LAWVPGELKL | 12.000 |
| 17 | 449 | ACAPSCAGDL | 12.000 |
| 18 | 210 | APSVDFQQSV | 12.000 |
| 19 | 433 | APRVKCNPSN | 12.000 |
| 20 | 492 | KPPSSFKMSI | 8.000 |
| 21 | 19 | WSSQYPLQSL | 6.000 |
| 22 | 347 | STPWQPPTVL | 6.000 |
| 23 | 429 | TPAKAPRVKC | 4.500 |
| 24 | 316 | NSTFYVKSSL | 4.000 |
| 25 | 58 | SSAPNMEHVL | 4.000 |
| 26 | 551 | VKRRLDSSCL | 4.000 |
| 27 | 721 | FCGPEHSTEL | 4.000 |
| 28 | 526 | SPRKALIPVS | 4.000 |
| 29 | 654 | NSSPENKNWL | 4.000 |
| 30 | 631 | ESCGTLPLPL | 4.000 |
| 31 | 582 | ENLHLDLCCL | 4.000 |
| 32 | 655 | SSPENKNWLL | 4.000 |
| 33 | 397 | KPGGDKLSTV | 4.000 |
| 34 | 16 | RNGWSSQYPL | 4.000 |
| 35 | 20 | SSQYPLQSLL | 4.000 |
| 36 | 277 | LILDSTGSTL | 4.000 |
| 37 | 579 | GQVENLHLDL | 4.000 |
| 38 | 294 | NIYMFNMTGL | 4.000 |
| 39 | 134 | GTCKGHQCSL | 4.000 |
| 40 | 641 | RPCGEGSEMV | 4.000 |
| 41 | 589 | CCLAGNQEDL | 4.000 |
| 42 | 323 | SSLSPDDQFL | 4.000 |
| 43 | 3 | FNSALRQPQL | 4.000 |
| 44 | 629 | ASESCGTLPL | 3.600 |
| 45 | 288 | ANCTDDNIYM | 3.000 |
| 46 | 439 | NPSNSSPSSA | 2.000 |
| 47 | 702 | TPSSMRKICT | 2.000 |
| 48 | 480 | INRRGSVSSV | 2.000 |
| 49 | 230 | LVSAGAVDGI | 2.000 |
| 50 | 620 | EPPSPISPYA | 2.000 |

TABLE XVI
Scoring Results 83P5G4 HLA PEPTIDES B35 9-MERS

| | | | Score (Estimate of Half Time of |
|---|---|---|---|
| | Start | Subsequence Residue | Disassociation of a Molecule |
| Rank | Position | Listing | Containing This Subsequence |
| 1 | 641 | RPCGEGSEM | 120.000 |
| 2 | 620 | EPPSPISPY | 40.000 |
| 3 | 148 | FSKFEKAVF | 22.500 |
| 4 | 348 | TPWQPPTVL | 20.000 |
| 5 | 264 | YPGSSTRKL | 20.000 |
| 6 | 255 | EPIASKSFL | 20.000 |
| 7 | 526 | SPRKALIPV | 12.000 |
| 8 | 704 | SSMRKICTY | 10.000 |
| 9 | 569 | KSCNCVTEL | 10.000 |
| 10 | 266 | GSSTRKLGY | 10.000 |
| 11 | 655 | SSPENKNWL | 10.000 |
| 12 | 516 | TPPASETKI | 8.000 |
| 13 | 472 | SPAKARSPI | 8.000 |
| 14 | 241 | KVWDLRKNY | 8.000 |
| 15 | 433 | APRVKCNPS | 6.000 |
| 16 | 335 | GSSDEAAYI | 6.000 |
| 17 | 289 | NCTDDNIYM | 6.000 |
| 18 | 628 | YASESCGTL | 6.000 |
| 19 | 658 | ENKNWLLAM | 6.000 |
| 20 | 676 | SPRSPSSQT | 6.000 |
| 21 | 244 | DLRKNYTAY | 6.000 |
| 22 | 656 | SPENKNWLL | 6.000 |
| 23 | 397 | KPGGDKLST | 6.000 |
| 24 | 517 | PPASETKIM | 6.000 |
| 25 | 35 | SGNDEHTSY | 6.000 |
| 26 | 116 | AAGDQTAKF | 6.000 |
| 27 | 20 | SSQYPLQSL | 5.000 |
| 28 | 489 | VSPKPPSSF | 5.000 |
| 29 | 323 | SSLSPDDQF | 5.000 |
| 30 | 4 | NSALRQPQL | 5.000 |
| 31 | 369 | CPSDFTKIA | 4.000 |
| 32 | 171 | RCNKKDGFY | 4.000 |
| 33 | 492 | KPPSSFKMS | 4.000 |
| 34 | 417 | RPGLVTVTS | 4.000 |
| 35 | 478 | SPINRRGSV | 4.000 |
| 36 | 107 | VPGELKLVT | 4.000 |
| 37 | 654 | NSSPENKNW | 3.750 |
| 38 | 415 | ESRPGLVTV | 3.000 |
| 39 | 59 | SAPNMEHVL | 3.000 |
| 40 | 60 | APNMEHVLA | 3.000 |
| 41 | 325 | LSPDDQFLV | 3.000 |
| 42 | 598 | LSKDSLGPT | 3.000 |
| 43 | 158 | TGGRDGNIM | 3.000 |
| 44 | 135 | TCKGHQCSL | 3.000 |
| 45 | 334 | SGSSDEAAY | 3.000 |
| 46 | 205 | NSKGLAPSV | 3.000 |
| 47 | 288 | ANCTDDNIY | 3.000 |
| 48 | 450 | CAPSCAGDL | 3.000 |
| 49 | 210 | APSVDFQQS | 3.000 |
| 50 | 705 | SMRKICTYF | 3.000 |

TABLE XVII
Scoring Results 83P5G4 HLA PEPTIDES B35 10-MERS

| | | | Score (Estimate of Half Time of |
|---|---|---|---|
| | Start | Subsequence Residue | Disassociation of a Molecule |
| Rank | Position | Listing | Containing This Subsequence |
| 1 | 490 | SPKPPSSFKM | 120.000 |
| 2 | 516 | TPPASETKIM | 60.000 |
| 3 | 697 | SPVTITPSSM | 40.000 |
| 4 | 255 | EPIASKSFLY | 40.000 |
| 5 | 23 | YPLQSLLTGY | 40.000 |
| 6 | 451 | APSCAGDLPL | 20.000 |
| 7 | 48 | VPVPPFGCTF | 20.000 |
| 8 | 458 | LPLPSNTPTF | 20.000 |
| 9 | 348 | TPWQPPTVLL | 20.000 |
| 10 | 492 | KPPSSFKMSI | 16.000 |
| 11 | 546 | ESRNRVKRRL | 15.000 |
| 12 | 34 | CSGNDEHTSY | 15.000 |
| 13 | 686 | NSRRQSGKTL | 15.000 |
| 14 | 333 | VSGSSDEAAY | 15.000 |
| 15 | 655 | SSPENKNWLL | 10.000 |
| 16 | 322 | KSSLSPDDQF | 10.000 |
| 17 | 287 | FANCTDDNIY | 9.000 |
| 18 | 460 | LPSNTPTFSI | 8.000 |
| 19 | 397 | KPGGDKLSTV | 8.000 |
| 20 | 641 | RPCGEGSEMV | 8.000 |
| 21 | 323 | SSLSPDDQFL | 7.500 |
| 22 | 526 | SPRKALIPVS | 6.000 |
| 23 | 433 | APRVKCNPSN | 6.000 |
| 24 | 69 | VANEEGFVRL | 6.000 |
| 25 | 14 | VLRNGWSSQY | 6.000 |
| 26 | 335 | GSSDEAAYIW | 5.000 |
| 27 | 316 | NSTFYVKSSL | 5.000 |
| 28 | 364 | TSVCWCPSDF | 5.000 |
| 29 | 20 | SSQYPLQSLL | 5.000 |
| 30 | 631 | ESCGTLPLPL | 5.000 |
| 31 | 359 | HSQEVTSVCW | 5.000 |
| 32 | 58 | SSAPNMEHVL | 5.000 |
| 33 | 654 | NSSPENKNWL | 5.000 |
| 34 | 82 | ESQSFRKKCF | 5.000 |
| 35 | 19 | WSSQYPLQSL | 5.000 |
| 36 | 704 | SSMRKICTYF | 5.000 |
| 37 | 376 | IATCSDDNTL | 4.500 |
| 38 | 417 | RPGLVTVTSS | 4.000 |
| 39 | 107 | VPGELKLVTA | 4.000 |
| 40 | 369 | CPSDFTKIAT | 4.000 |
| 41 | 60 | APNMEHVLAV | 4.000 |
| 42 | 210 | APSVDFQQSV | 4.000 |
| 43 | 142 | SLKSVAFSKF | 3.000 |
| 44 | 104 | LAWVPGELKL | 3.000 |
| 45 | 268 | STRKLGYSSL | 3.000 |
| 46 | 234 | GAVDGIIKVW | 3.000 |
| 47 | 6 | ALRQPQLGVL | 3.000 |
| 48 | 288 | ANCTDDNIYM | 3.000 |
| 49 | 77 | RLYNTESQSF | 3.000 |
| 50 | 626 | SPYASESCGT | 3.000 |

1. A polynucleotide that encodes an 83P5G4-related protein, wherein the polynucleotide is selected from the group consisting of:
a) a polynucleotide consisting of the sequence as shown in SEQ ID NO: 1, wherein T can also be U;
b) a polynucleotide consisting of the sequence as shown in SEQ ID NO: 1, from nucleotide residue number 130 through nucleotide number 2322, wherein T can also be U;
c) a polynucleotide that encodes an 83P5G4-related protein whose sequence is encoded by the cDNAs contained in the plasmids designated p83P5G4-1 deposited with American Type Culture Collection as Accession No. PTA-1154;
d) a polynucleotide that encodes an 83P5G4-related protein that is at least 90% identical to the entire amino acid sequence shown in SEQ ID NO: 2; and
e) a polynucleotide that is fully complementary to a polynucleotide of any one of (a)-(d).

2. A polynucleotide of claim 1 that encodes the polypeptide sequence shown in SEQ ID NO: 2.

3. A fragment of a polynucleotide of claim 1 comprising:
a) at least 10 contiguous nucleotides from a polynucleotide having the sequence shown in SEQ ID NO: 1 from nucleotide residue number 1 through nucleotide residue number 879 of SEQ ID NO: 1; or,
b) at least 10 contiguous nucleotides from a polynucleotide having the sequence as shown in SEQ ID NO: 1 from nucleotide residue number 2134 through nucleotide residue number 2838 of SEQ ID NO: 1; or,
c) a polynucleotide whose starting base is in a range of 1-879 of FIG. 2 (SEQ ID NO: 1) and whose ending base is in a range of 880-2838 of FIG. 2 (SEQ ID NO: 1); or,
d) a polynucleotide whose starting base is in a range of 880-2133 of FIG. 2 (SEQ ID NO: 1) and whose ending base is in a range of 2134-2838 of FIG. 2 (SEQ ID NO: 1); or,
e) a polynucleotide whose starting base is in a range of 1-879 of FIG. 2 (SEQ ID NO: 1) and whose ending base is in a range of 2134-2838 of FIG. 2 (SEQ ID NO: 1); or,
f) a polynucleotide that is a fragment of the polynucleotide of (a)-(e) that is at least 10 nucleotide bases in length; or,
g) a polynucleotide that selectively hybridizes under stringent conditions to a polynucleotide of (a) -(f).
h) a polynucleotide that is fully complementary to a polynucleotide of any one of (a)-(g); wherein a range is understood to specifically disclose each whole unit position thereof.

4. A polynucleotide of claim 3 that encodes an 83P5G4-related protein, wherein the polypeptide includes an amino acid sequence selected from the group consisting of NTSD (residues 190-193 of SEQ ID NO: 2), NYTA (residues 248-251 of SEQ ID NO: 2), NCTD (residues 289-292 of SEQ ID NO: 2), NMTG (residues 299-302 of SEQ ID NO: 2), NSTF (residues 316-319 of SEQ ID NO: 2), STR (residues 268-270 of SEQ ID NO: 2), TRK (residues 269-271 of SEQ ID NO: 2), TLK (residues 384-386 of SEQ ID NO: 2), SQK (residues 410-412 of SEQ ID NO: 2), SQK (residues 535-537 of SEQ ID NO: 2), SIK (residues 468-470 of SEQ ID NO: 2), SPK (residues 490-492 of SEQ ID NO: 2), SFK (residues 496-498 of SEQ ID NO: 2), SIR (residues 500-502 of SEQ ID NO: 2), SPR (residues 526-528 of SEQ ID NO: 2)and SPR (residues 676-678 of SEQ ID NO: 2).

5. A polynucleotide of claim 3 that encodes an 83P5G4-related protein, wherein the polypeptide comprises an HLA class I A1, A2, A3, A24, B7, B27, B58, B62 supermotif, or an HLA class II DR supermotif set forth in Table IIIB or an Alexander pan DR binding epitope supermotif or an HLA DR3 motif.

6. A polynucleotide of any one of claims 1-4 that is labeled with a detectable marker.

7. A recombinant expression vector that contains a polynucleotide of any one of claims 1-4.

8. A host cell that contains an expression vector of claim 7.

9. A process for producing an 83P5G4-related protein comprising culturing a host cell of claim 8 under conditions sufficient for the production of the polypeptide and recovering the 83P5G4-related protein so produced.

10. An 83P5G4-related protein produced by the process of claim 9.

11. An isolated 83P5G4-related protein of at least six amino acids.

12. The 83P5G4-related protein of claim 11, wherein 83P5G4-related protein has the amino acid sequence shown in SEQ ID NO: 2.

13. An isolated 83P5G4-related protein of claim 11 that has an amino acid sequence which is exactly that of an amino acid sequence encoded by a polynucleotide selected from the group consisting of:
a) a polynucleotide consisting of the sequence as shown in SEQ ID NO: 1, wherein T can also be U;
b) a polynucleotide that encodes an 83P5G4-related protein whose sequence is encoded by the cDNAs contained in the plasmids designated p83P5G4-1 deposited with American Type Culture Collection as Accession No. PTA-1154;
c) a polynucleotide that encodes an 83P5G4-related protein that is at least 90% identical to the entire amino acid sequence shown in SEQ ID NO: 2;
d) a polynucleotide that is fully complementary to a polynucleotide of any one of (a)-(d).

14. An isolated 83P5G4-related protein of claim 13 that has an amino acid sequence which is exactly that of an amino acid sequence encoded by a polynucleotide, where T can be U, selected from the group consisting of:
a) a polynucleotide having the sequence as shown in SEQ ID NO: 1 from nucleotide residue number 1 through nucleotide residue number 879 of SEQ ID NO: 1; or,
b) a polynucleotide having the sequence as shown in SEQ ID NO: 1 from nucleotide residue number 130 through nucleotide residue number 879 of SEQ ID NO: 1; or,
c) a polynucleotide having the sequence as shown in SEQ ID NO: 1 from nucleotide residue number 2134 through nucleotide residue number 2838 of SEQ ID NO: 1; or,
d) a polynucleotide having the sequence as shown in SEQ ID NO: 1 from nucleotide residue number 2134 through nucleotide residue number 2322 of SEQ ID NO: 1; or,
e) a polynucleotide whose starting base is in a range of 1-879 of FIG. 2 (SEQ ID NO: 1) and whose ending base is in a range of 880-2838 of FIG. 2 (SEQ ID NO: 1); or,
f) a polynucleotide whose starting base is in a range of 130-879 of FIG. 2 (SEQ ID NO: 1) and whose ending base is in a range of 880-2322 of FIG. 2 (SEQ ID NO: 1); or,
g) a polynucleotide whose starting base is in a range of 880-2133 of FIG. 2 (SEQ ID NO: 1) and whose ending base is in a range of 2134-2838 of FIG. 2 (SEQ ID NO: 1); or,
h) a polynucleotide whose starting base is in a range of 880-2133 of FIG. 2 (SEQ ID NO: 1) and whose ending base is in a range of 2134-2322 of FIG. 2 (SEQ ID NO: 1); or,
i) a polynucleotide whose starting base is in a range of 130-879 of FIG. 2 (SEQ ID NO: 1) and whose ending base is in a range of 2134-2322 of FIG. 2 (SEQ ID NO: 1); or,
j) a polynucleotide of (a)-(i) that is more than 10 nucleotide bases in length; or
k) a polynucleotide that selectively hybridizes under stringent conditions to a polynucleotide of (a)-(j);
wherein a range is understood to specifically disclose each whole unit position thereof.

15. An antibody or fragment thereof that specifically binds to an 83P5G4-related protein.

16. The antibody or fragment thereof of claim 15, which is monoclonal.

17. A recombinant protein comprising the antigen-binding region of a monoclonal antibody of claim 16.

18. The antibody or fragment thereof of claim 16, which is labeled with a detectable marker.

19. The recombinant protein of claim 17, which is labeled with a detectable marker.

20. The antibody fragment of claim 15, which is an Fab, F(ab′)2, Fv or Sfv fragment.

21. The antibody of claim 15, which is a human antibody.

22. The recombinant protein of claim 19, which comprises murine antigen-binding region residues and human constant region residues.

23. A non-human transgenic animal that produces an antibody of claim 15.

24. A hybridoma that produces an antibody of claim 15.

25. A single chain monoclonal antibody that comprises the variable domains of the heavy and light chains of a monoclonal antibody of claim 21.

26. A vector comprising a polynucleotide encoding a single chain monoclonal antibody of claim 25 that immunospecifically binds to an 83P5G4-related protein.

27. An assay for detecting the presence of an 83P5G4-related protein or polynucleotide in a biological sample comprising: contacting the sample with an antibody or polynucleotide, respectively, that specifically binds to the 83P5G4-related protein or polynucleotide, respectively, and detecting the binding of 83P5G4-related protein or polynucleotide, respectively, in the sample thereto.

28. An assay of claim 27 for detecting the presence of an 83P5G4-related protein or polynucleotide comprising the steps of: obtaining a sample, evaluating said sample in the presence of an 83P5G4-related protein or polynucleotide, whereby said evaluating step produces a result that indicates the presence or amount of 83P5G4-related protein or polynucleotide, respectively.

29. An assay of claim 28 for detecting the presence of a 83P5G4 polynucleotide in a biological sample, comprising:
a) contacting the sample with a polynucleotide probe that specifically hybridizes to a polynucleotide encoding an 83P5G4-related protein having an amino acid sequence shown in FIG. 2; and
b) detecting the presence of a hybridization complex formed by the hybridization of the probe with 83P5G4 polynucleotide in the sample, wherein the presence of the hybridization complex indicates the presence of 83P5G4 polynucleotide within the sample.

30. An assay for detecting the presence of 83P5G4 mRNA in a biological sample comprising:
a) producing cDNA from the sample by reverse transcription using at least one primer;
b) amplifying the cDNA so produced using 83P5G4 polynucleotides as sense and antisense primers to amplify 83P5G4 cDNAs therein;
c) detecting the presence of the amplified 83P5G4 cDNA,
wherein the 83P5G4 polynucleotides used as the sense and antisense probes are capable of amplifying the 83P5G4 cDNA contained within the plasmid as deposited with American Type Culture Collection as Accession No. PTA-1154.

31. A method of claim 30 for monitoring 83P5G4 gene products comprising:
determining the status of 83P5G4 gene products expressed by cells in a tissue sample from an individual;
comparing the status so determined to the status of 83P5G4 gene products in a corresponding normal sample; and
identifying the presence of aberrant 83P5G4 gene products in the sample relative to the normal sample.

32. The method of claim 31, wherein the 83P5G4 gene products are monitored by comparing the polynucleotide sequences of 83P5G4 gene products in the test tissue sample with the polynucleotide sequences of 83P5G4 gene products in a corresponding normal sample.

33. The method of claim 31, wherein the 83P5G4 gene products are monitored by comparing the levels 83P5G4 gene products in the test tissue sample with the levels of 83P5G4 gene products in the corresponding normal sample.

34. A method of diagnosing the presence of cancer in an individual comprising: performing the method of claim 32 or 33 whereby the presence of elevated 83P5G4 mRNA or protein expression in the test sample relative to the normal tissue sample provides an indication of the presence of cancer.

35. The method of claim 34, wherein the cancer occurs in a tissue set forth in Table I.

36. Use of an 83P5G4-related protein, a vector comprising a polynucleotide encoding a single chain monoclonal antibody that immunospecifically binds to an 83P5G4-related protein, an antisense polynucleotide complementary to a polynucleotide having 83P5G4 coding sequences, or a ribozyme capable of cleaving a polynucleotide having 83P5G4 coding sequences, for the preparation of a composition for treating a patient with a cancer that expresses 83P5G4.

37. The use of claim 36, wherein the cancer occurs in a tissue set forth in Table I.

38. A pharmaceutical composition comprising an 83P5G4-related protein, an antibody or fragment thereof that specifically binds to an 83P5G4-related protein, a vector comprising a polynucleotide encoding a single chain monoclonal antibody that immunospecifically binds to an 83P5G4-related protein, a polynucleotide comprising an 83P5G4-related protein coding sequence, an antisense polynucleotide complementary to a polynucleotide having an 83P5G4 coding sequences or a ribozyme capable of cleaving a polynucleotide having 83P5G4 coding sequences and, optionally, a physiologically acceptable carrier.

39. A method of treating a patient with a cancer that expresses 83P5G4 which comprises administering to said patient a composition of claim 38 comprising a vector that comprises a polynucleotide encoding a single chain monoclonal antibody that immunospecifically binds to an 83P5G4-related protein, such that the vector delivers the single chain monoclonal antibody coding sequence to the cancer cells and the encoded single chain antibody is expressed intracellularly therein.

40. A method of inhibiting the development of a cancer expressing 83P5G4 in a patient, comprising administering to the patient an effective amount of the vaccine composition of claim 38.

41. A method of generating an immune response in a mammal comprising exposing the mammal's immune system to an immunogenic portion of an 83P5G4-related protein of claim 38, so that an immune response is generated to 83P5G4.

42. A method of delivering a cytotoxic agent to a cell that expresses 83P5G4 comprising conjugating the cytotoxic agent to an antibody or fragment thereof of claim 15 that specifically binds to an 83P5G4 epitope and exposing the cell to the antibody-agent conjugate.

43. A method of inducing an immune response to a 83P5G14 protein, said method comprising:
providing an 83P5G4-related protein T cell or B cell epitope;
contacting the epitope with an immune system T cell or B cell respectively, whereby the immune system T cell or B cell is induced.

44. The method of claim 43, wherein the immune system cell is a B cell, whereby the induced B cell generates antibodies that specifically bind to the 83P5G4-related protein.

45. The method of claim 43, wherein the immune system cell is a T cell that is a cytotoxic T cell (CTL), whereby the activated CTL kills an autologous cell that expresses the 83P5G4 protein.

46. The method of claim 43, wherein the immune system cell is a T cell that is a helper T cell (HTL), whereby the activated HTL secretes cytokines that facilitate the cytotoxic activity of a CTL or the antibody producing activity of a B cell.
